(19) United States
(12) Patent Application Publication
(10) Pub. No.: US 2005/0059093 A1
(43) Pub. Date: Mar. 17, 2005

(54) Method for detecting modulators of Notch signalling

(76) Inventors: Mark William Bodmer, Cambridge (GB); Emmanuel Cyrille Pascal Briend, Cambridge (GB); Brian Robert Champion, Cambridge (GB); Grahame James McKenzie, Cambridge (GB); Tamara Tugal, Cambridge (GB); George Albert Ward, Cambridge (GB); Lesley Lynn Young, Cambridge (GB)

Correspondence Address: FROMMER LAWRENCE & HAUG, 745 FIFTH AVENUE- 10TH FL., NEW YORK, NY, 10151, US

(21) Appl. No.: 10/764,415

(22) Filed: Jan. 23, 2004

Related U.S. Application Data
(63) Continuation-in-part of application No. PCT/GB02/03397, filed on Jul. 25, 2002.

(30) Foreign Application Priority Data
Jul. 25, 2001 (GB) 0118153.6
Apr. 5, 2002 (GB) 0207930.9
May 28, 2002 (GB) 0212282.8
May 28, 2002 (GB) 0212283.6

(51) Int. Cl. G01N 33/53; G01N 33/567

(52) U.S. Cl. 435/7.2

(57) ABSTRACT

A method for detecting modulators of Notch signalling is described. The method includes the step of monitoring Notch signalling in a cell of the immune system in the presence of a candidate modulator.

### CROSS REFERENCE TO RELATED APPLICATIONS

This application is a continuation-in-part of International Application No. PCT/GB02/03397, filed on Jul. 25, 2002, published as WO 03/012441 on Feb. 13, 2003, and claiming priority to GB applications Ser. Nos. 0118153.6, filed on Jul. 25, 2001, 0207930.9, filed on Apr. 5, 2002, 0212282.8, filed on May 28, 2002 and 0212283.6, filed on May 28, 2002. Reference is made to U.S. application Ser. No. 09/310,685, filed on May 4, 1999, Ser. No. 09/870,902, filed on May 31, 2001, Ser. No. 10/013,310, filed on Dec. 7, 2001, Ser. No. 10/147,354, filed on May 16, 2002, Ser. No. 10/357,321, filed on Feb. 3, 2002, Ser. No. 10/682,230, filed on Oct. 9, 2003 and Ser. No. 10/720,896, filed on Nov. 24, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a method of and assay for detecting modulators of Notch signalling. The present invention also relates to novel modulators identifiable by such a method and uses thereof in therapy. The present invention further relates to a composition comprising at least one such modulator.

### BACKGROUND OF THE INVENTION

Notch signal transduction plays a critical role in cell fate determination in vertebrate and invertebrate tissues. Notch is expressed at many stages of Drosophila embryonic and larval development and in many different cells implying a wide range of functions including an important role in neurogenesis and in the differentiation of mesodermal and endodermal cells. There are at least four mammalian Notch genes (Notch-1, Notch-2, Notch-3 and Notch-4). Notch-I, which most closely resembles the proteins of invertebrates and lower vertebrates, is widely expressed and is essential for early development. Recent evidence suggests that Notch signalling contributes to lineage commitment of immature T-cells in the thymus.

During maturation in the thymus, T-cells acquire the ability to distinguish self-antigens from those that are non-self, a process termed “self tolerance”. Tolerance to a non-self antigen, however, may be induced by immunisation under specific conditions with a peptide fragment comprising that antigen. In autoimmune diseases such as multiple sclerosis, rheumatoid arthritis or diabetes, there is a failure of the proper regulation of tolerance. Improved treatment methods for re-establishing tolerance are desirable for autoimmune diseases. Similarly in allergic conditions and for transplantation of an organ or tissue from a donor individual, induction of tolerance to particular foreign antigens or profiles of foreign antigens is desirable.

The expression on the cell surface of normal adult cells of the peripheral immune system of Notch and its ligands, Delta and Serrate, suggests a role for these proteins in T-cell acquired immunocompetence. T-cells express Notch-1 mRNA constitutively. Delta expression is limited to only a subset of T-cells in the peripheral lymphoid tissues. Serrate expression is restricted to a subset of antigen presenting cells (APCs). These observations reinforce the view that the Notch receptor ligand family continues to regulate cell fate decisions in the immune system beyond embryonic development with Notch signalling playing a central role in the induction of peripheral unresponsiveness (tolerance or anergy), linked suppression and infectious tolerance (Hoyne et al.).

Thus, as described in WO 98/20142, manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also recently been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins. Delta or Serrate induced T cells specific to one antigenic epitope are also able to transfer tolerance to T cells recognising other epitopes on the same or related antigens, a phenomenon termed “epitope spreading”.

Notch ligand expression also plays a role in cancer. Indeed, upregulated Notch ligand expression has been observed in some tumour cells. These tumour cells are capable of rendering T cells unresponsive to restimulation with a specific antigen, thus providing a possible explanation of how tumour cells prevent normal T cell responses. By downregulating Notch signalling in vivo in T cells, it may be possible to prevent tumour cells from inducing immunotolerance in those T cells that recognise tumour-specific antigens. In turn, this would allow the T cells to mount an immune response against the tumour cells (WO00/135990).

A description of the Notch signalling pathway and conditions affected by it may be found in our published PCT Applications WO 98/20142, WO 00/36089 and WO 01/35990. The text of each of PCT/GB97/03058 (WO 98/20142), PCT/GB99/04233 (WO 00/36089) and PCT/GB00/04391 (WO 0135990) is hereby incorporated herein by reference.

There remains a need in the art for the provision of further diagnostic or therapeutic compositions useful in the detection, prevention and treatment of diseases or conditions of, or relating to, the immune system, and in particular, but not exclusively, T cell mediated diseases or disorders. The present invention addresses this problem by delivering an effective method of identifying novel modulators of the Notch signalling pathway. While many assay methods are known in the art, the present invention is based in our knowledge of the Notch signalling pathway and realisation that an effective assay method for detection of novel modulators needs to be carried out using a cell of the immune system.

### SUMMARY OF THE INVENTION

According to one aspect of the present invention there is provided a method for detecting modulators of Notch signalling comprising the step of monitoring Notch signalling in a cell of the immune system in the presence and absence of a candidate modulator and determining whether the candidate modulator modulates Notch signalling.

According to a further aspect of the present invention there is provided a method for detecting modulators of Notch signalling comprising the steps of:
(a) contacting a cell of the immune system with a candidate modulator;
(b) monitoring Notch signalling; and
(c) determining whether the candidate modulator modulates Notch signalling.
   “Contacting” means bringing together in such a way so as the cell may interact with the candidate modulator. Preferably this will be in an aqueous solvent or buffering solution.

According to a further aspect of the invention there is provided a method for detecting modulators of Notch or immune signalling comprising the steps of (in any order):
(a) activating Notch signalling in a cell of the immune system;
(b) contacting the cell with a candidate modulator of Notch or immune signalling;
(c) monitoring Notch or immune signalling; and
(d) determining whether the candidate modulator modulates Notch or immune signalling.

According to a further aspect of the invention there is provided a method for detecting modulators of Notch or immune signalling comprising the steps of (in any order):
(a) activating a cell of the immune system;
(b) contacting the cell with a candidate modulator of Notch or immune signalling;
(c) monitoring Notch or immune signalling; and
(d) determining whether the candidate modulator modulates Notch or immune signalling.

According to a further aspect of the invention there is provided a method for detecting modulators of Notch or immune signalling comprising the steps of (in any order):
(a) activating a cell of the immune system;
(b) activating Notch signalling in the cell;
(c) contacting the cell with a candidate modulator of Notch or immune signalling;
(d) monitoring Notch or immune signalling; and
(e) determining whether the candidate modulator modulates Notch or immune signalling.

According to a further aspect of the invention there is provided a method for detecting modulators of Notch signalling comprising the steps of (in any order):
(a) activating Notch signalling in a cell of the immune system;
(b) contacting the cell with a candidate modulator of Notch signalling;
(c) monitoring Notch or immune signalling; and
(d) determining whether the candidate modulator modulates Notch or immune signalling.

According to a further aspect of the invention there is provided a method for detecting modulators of Notch signalling comprising the steps of (in any order):
(a) activating a cell of the immune system;
(b) contacting the cell with a candidate modulator of Notch signalling;
(c) monitoring Notch or immune signalling; and
(d) determining whether the candidate modulator modulates Notch or immune signalling.

According to a further aspect of the invention there is provided a method for detecting modulators of Notch signalling comprising the steps of (in any order):
(a) activating a cell of the immune system;
(b) activating Notch signalling in the cell;
(c) contacting the cell with a candidate modulator of Notch signalling;
(d) monitoring Notch or immune signalling; and
(e) determining whether the candidate modulator modulates Notch or immune signalling.

Suitably immune cell activation is at least 20%, preferably at least 70% optimal with respect to Notch or immune signalling.

The candidate modulator may be any organic or inorganic compound. Preferably the candidate modulator is selected from a group consisting of: small natural or synthetic molecule compounds, a polypeptide, a polynucleotide, an antibody or a fragment of an antibody and a cytokine or a fragment of a cytokine.

In a preferred embodiment, the step of monitoring Notch signalling comprises the steps of monitoring levels of expression of at least one target gene. The target gene may be an endogenous target gene of the Notch signalling pathway or a reporter gene.

Known endogenous target genes of the Notch signalling pathway include Deltex, Hes-1, Hes-5, E(spl), Il-10, CD-23, Dlx-1, CTLA4, CD-4, Numb, Mastermind and Dsh.

Many reporter genes are standard in the art and include genes encoding an enzymatic activity, genes comprising a radiolabel or a fluorescent label and genes encoding a predetermined polypeptide epitope.

Preferably at least one target gene is under the transcriptional control of a promoter region sensitive to Notch signalling. Even more preferably, at least one target gene is under the transcriptional control of a promoter region sensitive to Notch signalling and a second signal, and/or a third signal wherein the second and third signals are different.

An example of a signal of use in the present invention is a signal that results from activation of a signalling pathway specific to cells of the immune system, such as a T cell receptor (TCR) signalling pathway, a B cell receptor (BCR) signalling pathway or a Toll-like receptor (TLR) signalling pathway, with or without an accessory signal (known in the art as costimulatory signals for T and B cell receptor signalling).

Another example of a signal of use in the present invention is a costimulus specific to cells of the immune system such as B7 proteins, including B7.1-CD80, B7.2-CD86, B7H1, B7H2, B7H3, B7RP1, B7RP2, CTLA4, ICOS, CD2, CD24, CD27, CD28, CD30, CD34, CD38, CD40, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, TRANCE/RANK ligands, Fas ligand, MHC class II, DEC205-CD205, CD204-Scavenger receptor, CD14, CD206 (mannose receptor), Toll-like receptors (TLR) such as TLR 1-9, CD207 (Langerin), CD209 (DC-SIGN), FCγreceptor 2 (CD32), CD64 (FCγ receptor 1), CD68, CD83, CD33, CD54, BDCA-2, BDCA-3, BDCA-4, chemokine receptors, cytokines, growth factors or growth factor receptor agonists, and variants, derivatives, analogues and fragments thereof.

In a preferred embodiment, the method of the present invention is carried out in a T cell or T cell progenitor or an antigen presenting cell (APC). APCs are cells which are capable of expressing MHC class II molecules and able to present antigens to CD4+ T cells. Preferably, the APC will be a myeloid lineage cell such as a dendritic cell, for example a Langerhans cell, a monocyte or macrophage or a primary cell or a B lineage cell.

Levels of expression of at least one target gene can be monitored with a protein or a nucleic acid assay.

In accordance with another aspect of the present invention there is provided a method for detecting modulators of Notch signalling comprising the steps of:
(a) activating a cell of the immune system;
(b) contacting the cell with a candidate modulator;
(c) monitoring Notch signalling;
   (wherein steps (a), (b) and (c) can be carried out in any order); and
(d) determining whether the candidate modulator modulates Notch signalling.

Suitably the expression of the at least one target gene is monitored with a protein or nucleic acid assay

Suitably the candidate modulator has a molecular weight of less than about 1000, suitably less than about 500.

Preferably the cell of the immune system is a T-cell or T-cell progenitor.

Preferably the T-cell is activated by activation of the T-cell receptor.

Preferably the T-cell receptor is activated with an antigen or antigenic determinant.

Preferably the T-cell receptor is activated by an anti-CD3 or anti-TCR antibody which are preferably bound to a support. Preferably the anti-CD3 or anti-TCR antibody is bound to a particulate support.

Preferably the T-cell is co-activated, suitably by activation of CD28.

Preferably the T-cell receptor is co-activated by an anti-CD28 antibody or CD28 ligand, such as an active domain of B7.

Preferably the T-cell is activated by an anti-CD3 antibody and co-activated by anti-CD28 antibody.

Alternatively the T-cell may be activated with a calcium ionophore or an activator of protein kinase C or MAP Kinase.

Suitably the immune cell may be transfected with an expression vector coding for Notch, a constitutively active truncated form of Notch or a Notch IC domain, an if desired a Notch reporter construct.

In an preferred embodiment the method comprises the steps of:
i) activating Notch signalling in the immune cell with a further agent; and
ii) determining whether the candidate modulator modulates such Notch signalling activation and/or immune cell activation.

In one embodiment Notch signalling may be activated with a Notch ligand or an active portion of a Notch ligand, for example a Notch ligand EC domain. Suitably the Notch ligand may be bound to a membrane or support.

According to a further aspect of the present invention there is provided a particle comprising an active portion of a Delta ligand bound to a particulate support matrix.

Preferably the particulate support matrix is a bead. The bead may be, for example, a magnetic bead (eg as available under the trade name “Dynal”) or a polymeric bead such as a Sepharose bead. Suitably a plurality of active portions of a Delta ligand are bound to the particulate support matrix.

According to a yet further aspect of the present invention there is provided a modulator identifiable or identified by the method of the invention.

According to yet another aspect of the present invention there is provided the use of a modulator according to the present invention in the preparation of a medicament for the treatment of a disease or condition of, or related to the immune system. Preferably, the disease is a T-cell mediated disease.

According to yet another aspect of the present invention there is provided a pharmaceutical composition comprising a therapeutically effective amount of at least one modulator according to the invention and a pharmaceutically acceptable carrier, diluent and/or excipient.

Preferably Notch signalling pathway is activated with an agent capable of activating a Notch receptor. Suitably the modulator is a Notch ligand or a biologically active fragment or derivative of a Notch ligand. The Notch ligand may be soluble or presented on a cell or cell membrane, or bound to a support.

Suitably the modulator of the Notch signalling pathway may comprise or code for a fusion protein. For example, the modulator may comprise or code for a fusion protein comprising a segment of a Notch ligand extracellular domain and an immunoglobulin F_{c} segment.

Suitably the modulator of the Notch signalling pathway comprises or codes for a protein or polypeptide comprising a Notch ligand DSL domain and at least one EGF domain or a fragment, derivative, homologue, analogue or allelic variant thereof.

Preferably the modulator of the Notch signalling pathway comprises or codes for a Notch ligand DSL domain and at least 1 to 20, suitably at least 3 to 15, for example at least 3 to 8 EGF repeat motifs. Suitably the DSL and EGF sequences are or correspond to mammalian sequences. Preferred sequences include human sequences.

According to a further aspect of the invention there is provided a particle comprising protein comprising a Delta DSL domain and at least one Delta EGF domain bound to a particulate support matrix. Suitably the protein comprisies a Delta extracellular domain or an active portion thereof bound to a particulate support matrix. In one embodiment the particulate support matrix is a bead. Preferably a plurality of such proteins are bound to the particulate support matrix.

Alternatively or in addition the modulator of the Notch signalling pathway may comprise a Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

Suitably the modulator of the Notch signalling pathway comprises Delta or a fragment, derivative, homologue, analogue or allelic variant thereof or a polynucleotide encoding Delta or a fragment, derivative, homologue, analogue or allelic variant thereof.

Alternatively or in addition the modulator of the Notch signalling pathway may comprise Serrate/Jagged or a fragment, derivative, homologue, analogue or allelic variant thereof or a polynucleotide encoding Serrate/Jagged or a fragment, derivative, homologue, analogue or allelic variant thereof.

Alternatively or in addition the modulator of the Notch signalling pathway may comprise Notch or a fragment, derivative, homologue, analogue or allelic variant thereof or a polynucleotide encoding Notch or a fragment, derivative, homologue, analogue or allelic variant thereof.

Alternatively or in addition the modulator of the Notch signalling pathway may comprise a dominant negative version of a Notch signalling repressor, or a polynucleotide which codes for a dominant negative version of a Notch signalling repressor.

Alternatively or in addition the modulator of the Notch signalling pathway may comprise a polypeptide capable of upregulating the expression or activity of a Notch ligand or a downstream component of the Notch signalling pathway, or a polynucleotide which codes for such a polypeptide.

Suitably the modulator of the Notch signalling pathway may comprise an antibody, antibody fragment or antibody derivative or a polynucleotide which codes for an antibody, antibody fragment or antibody derivative.

According to a further aspect of the invention there is provided a method for detecting genes which are upregulated in an immune cell in response to a combination of Notch signalling and immune cell activation comprising the steps of (in any order):
(a) activating an immune cell;
(b) activating Notch signalling in the cell;
(c) monitoring gene expression; and
(d) determining which genes are upregulated or downregulated.

According to a further aspect of the invention there is provided a method for detecting genes which are more significantly upregulated or downregulated in an immune cell in response to a combination of Notch signalling and immune cell activation than in response to Notch signalling or immune cell activation alone comprising the steps of (in any order):
(a) activating an immune cell;
(b) activating Notch signalling in the cell;
(c) monitoring gene expression;
(d) determining whether gene expression is upregulated or downregulated in the cell; and
(e) comparing gene expression from step (d) with controls in which the cell is not activated or Notch signalling is not activated.

In one embodiment gene expression may be monitored using a microarray and preferably the immune cell is a T-cell.

According to a further aspect of the invention there is provided a gene detected by a method as defined above.

According to a further aspect of the invention there is provided the use of a modulator of a gene as detected by a method described above for the treatment of an immune disorder.

According to a further aspect of the invention there is provided an assay comprising the steps of (in any order):
(a) providing a culture of immune cells;
(b) transfecting said cells with a Notch signalling reporter construct;
(c) optionally transfecting said cells with a nucleic acid coding for Notch, a constitutively active truncated form of Notch or a Notch IC domain;
(d) optionally providing a Notch ligand;
(e) exposing the cells to one or more compound(s) to be tested; and
(f) determining the difference in Notch signalling between cells exposed to the compound(s) to be tested and cells not so exposed.

According to a further aspect of the invention there is provided an assay comprising the steps of (in any order):
(a) providing a culture of immune cells;
(b) optionally transfecting said cells with a Notch signalling reporter construct;
(c) transfecting said cells with a nucleic acid coding for Notch, a constitutively active truncated form of Notch or a Notch IC domain;
(d) optionally providing a Notch ligand;
(e) exposing the cells to one or more compound(s) to be tested; and
(f) determining the difference in Notch signalling between cells exposed to the compound(s) to be tested and cells not so exposed.

Preferably the assay comprises the further step of activating the immune cell.

Suitablyly Notch signalling may be monitored by monitoring cytokine production, for example by monitoring IL-10, TNF, IFN, IL-5, or IL-13 production.

According to a further aspect of the invention there is provided an immune cell transfected with:
(i) a Notch signalling reporter construct; and
(ii) an expression vector coding for Notch, a constitutively active truncated form of Notch or a Notch IC domain.

According to a further aspect of the invention there is provided an immune cell transfected with:
(i) a Notch signalling reporter construct; and
(ii) an expression vector coding for a constitutively active truncated form of Notch.

According to a further aspect of the invention there is provided an immune cell transfected with:
(i) a Notch signalling reporter construct; and
(ii) an expression vector coding for a Notch IC domain.

Preferbly the immune cell is stably transfected.

According to a further aspect of the invention there is provided a method for detecting modulators of Notch signalling comprising the steps of monitoring Notch signalling in a cell of the immune system in the presence and absence of a candidate modulator having a molecular weight of less than about 1000, and determining whether the candidate modulator modulates Notch signalling.

According to a further aspect of the invention there is provided a method for detecting modulators of Notch signalling comprising the steps of:
(a) contacting a cell of the immune system with a candidate modulator having a molecular weight of less than about 1000;
(b) monitoring Notch signalling; and
(c) determining whether the candidate modulator modulates Notch signalling.

Preferably the candidate modulator has a molecular weight of less than about 500.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 shows a schematic representation of the Notch signalling pathway

FIG. 2 shows a schematic representation of the Notch signalling pathway;

FIG. 3 shows a schematic representation of Notch and examples of immune cell signalling pathways which may be used in screening for immune cell modulators of Notch signalling;

FIGS. 4 shows a schematic representation of the assays of Examples 1 to 9;

FIG. 5 shows a schematic representation of the assays of Examples 1 to 9;

FIG. 6 shows a schematic representation of the assays of Examples 1 to 9;

FIG. 7 shows the results of Example 3;

FIG. 8 shows the results of Example 4;

FIG. 9 shows the results of Example 5;

FIG. 10 shows the results of Example 6;

FIG. 11 shows the results of Example 7;

FIG. 12 shows the results of Example 8;

FIG. 13 shows the results of Example 10;

FIGS. 14A and 14B show the results of Example 11;

FIGS. 15A and 15B show the results of Example 11;

FIG. 16 shows the results of Example 11;

FIG. 17 shows the results of Example 11;

FIG. 18 shows the results of Example 11;

FIG. 19 shows the results of Example 12;

FIGS. 20A and 20B show the results of Example 13;

FIG. 21 shows the results of Example 14;

FIGS. 22A and 22B illustrate the results of Example 15;

FIG. 23 shows the results of Example 15;

FIGS. 24A and 24B illustrate the results of Example 15;

FIGS. 25A and 25B show the results of Example 15;

FIGS. 26A, 26B and 26C show the results of Example 15;

FIGS. 27 shows the results of Example 16;

FIGS. 28 shows the results of Example 16;

FIG. 29 shows the results of Example 17;

FIGS. 30A and 30B show the results of Example 18;

FIG. 31 shows schematic representations of the Notch ligands Jagged and Delta;

FIG. 32 shows aligned amino acid sequences of DSL domains from various Drosophila and mammalian Notch ligands;

FIG. 33 shows amino acid sequences of human Delta-1, Delta-3 and Delta-4;

FIG. 34 shows amino acid sequences of human Jagged-1 and Jagged-2;

FIG. 35 shows the amino acid sequence of human Notch1;

FIG. 36 shows the amino acid sequence of human Notch2;

FIG. 37 shows schematic representations of Notch 1-4; and

FIG. 38 shows a schematic representation of NotchIC.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Notch Signalling

As used herein, the expression “Notch signalling” is synonymous with the expression “the Notch signalling pathway” and refers to any one or more of the upstream or downstream events that result in, or from, (and including) activation of the Notch receptor.

Notch signalling directs binary cell fate decisions in the embryo. Notch was first described in Drosophila as a transmembrane protein that functions as a receptor for two different ligands, Delta and Serrate. Vertebrates express multiple Notch receptors and ligands. At least four Notch receptors (Notch-1, Notch-2, Notch-3 and Notch-4) have been identified to date in human cells.

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs and is mediated by a furin-like convertase.

Notch receptors are inserted into the membrane as disulphide-linked heterodimeric molecules consisting of an extracellular domain containing up to 36 epidermal growth factor (EGF)-like repeats and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and, like the ankyrin-like repeats, is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the NotchIC for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

NotchIC processing occurs only in response to binding of Notch ligands Delta or Serrate/Jagged. The post-translational modification of the nascent Notch receptor in the Golgi (Munro; Ju) appears, at least in part, to control which of the two types of ligand it interacts with on a cell surface. The Notch receptor is modified on its extracellular domain by Fringe, a glycosyl transferase enzyme that binds to the Notch/Lin motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially interact with Delta (Panin; Hicks). Although Drosophila has a single Fringe gene, vertebrates are known to express multiple genes (Radical, Manic and Lunatic Fringes) (Irvine).

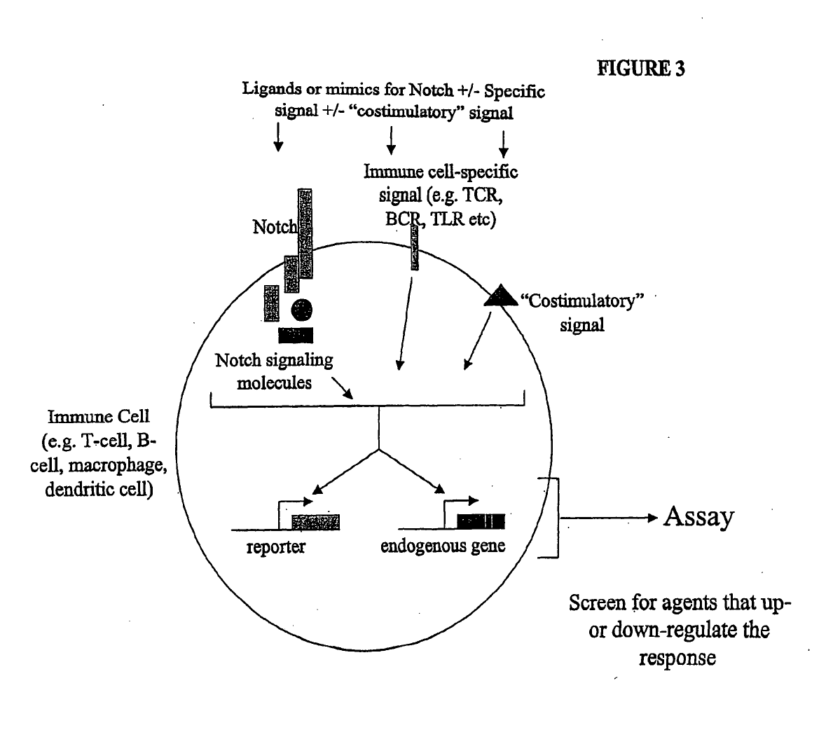

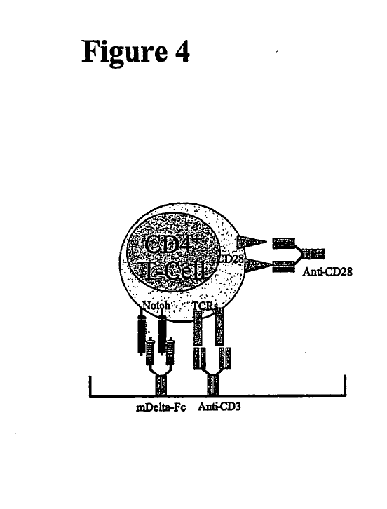

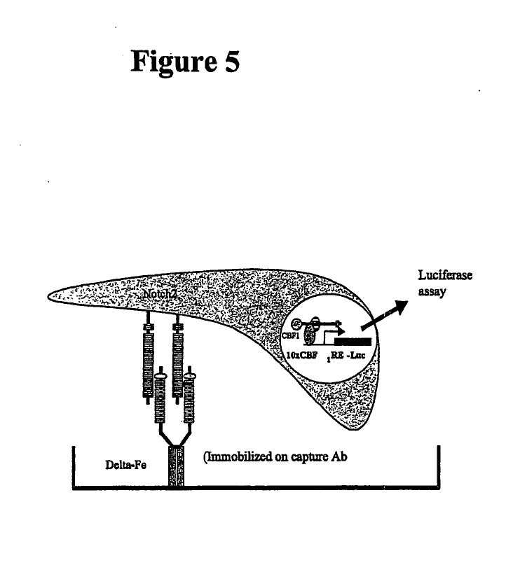

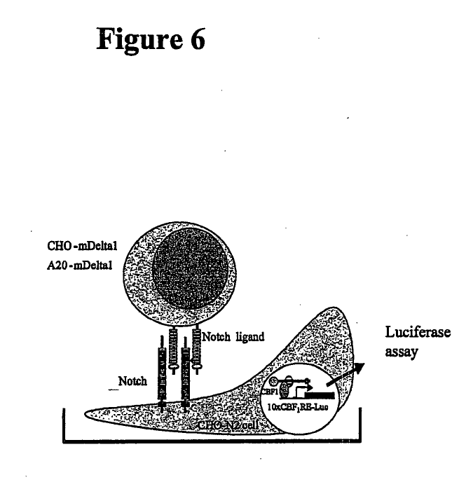

Thus, signal transduction from the Notch receptor can occur via different pathways. The better defined pathway involves proteolytic cleavage of the intracellular domain of Notch (NotchIC) that translocates to the nucleus and forms a transcriptional activator complex with the CSL family protein CBF1 (supressor of hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex (FIG. 3). Unlike CBF1, Deltex does not move to the nucleus following Notch activation but instead can interact with Grb2 and modulate the Ras-Jnk signalling pathway.

As described above, several endogenous modulators of Notch are already known. These include, for example, the Notch ligands Delta and Serrate. An aim of the present invention is the detection of novel Notch signalling modulators.

Candidate Modulators

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists.

The term “candidate modulator” is used to describe any one or more molecule(s) which may be, or is suspected of being, capable of functioning as a modulator of Notch signalling. Said molecules may for example be organic “small molecules” or polypeptides. Suitably, candidate molecules comprise a plurality of, or a library of such molecules or polypeptides. These molecules may be derived from known modulators. “Derived from” means that the candidate modulator molecules preferably comprise polypeptides which have been fully or partially randomised from a starting sequence which is a known modulator of Notch signalling. Most preferably, candidate molecules comprise polypeptides which are at least 40% homologous, more preferably at least 60% homologous, even more preferably at least 75% homologous or even more, for example 85% , or 90%, or even more than 95% homologous to one or more known Notch modulator molecules, using the BLAST algorithm with the parameters as defined herein.

The candidate modulator of the present invention may be an organic compound or other chemical. In this embodiment, the candidate modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In one preferred embodiment, the candidate compound will be an amino acid sequence or a chemical derivative thereof, or a combination thereof. In another preferred embodiment, the candidate compound will be a nucleotide sequence, which may be a sense sequence or an anti-sense sequence. The candidate modulator may also be an antibody.

The term “antibody” includes intact molecules as well as fragments thereof, such as Fab, F(ab′)2, Fv and scFv which are capable of binding the epitopic determinant. These antibody fragments retain some ability to selectively bind with its antigen or receptor and include, for example:
(i) Fab, the fragment which contains a monovalent antigen-binding fragment of an antibody molecule can be produced by digestion of whole antibody with the enzyme papain to yield an intact light chain and a portion of one heavy chain;
(ii) Fab′, the fragment of an antibody molecule can be obtained by treating whole antibody with pepsin, followed by reduction, to yield an intact light chain and a portion of the heavy chain; two Fab′ fragments are obtained per antibody molecule;
(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;
(iv) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

Modulators may be synthetic compounds or natural isolated compounds.

By a protein which is for Notch signalling transduction is meant a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the protein is a domain that allows activation of target genes of the Notch signalling pathway, or a polynucleotide sequence which codes therefor.

A very important component of the Notch signalling pathway is Notch receptor/Notch ligand interaction. Thus Notch signalling may involve changes in expression, nature, amount or activity of Notch ligands or receptors or their resulting cleavage products. In addition, Notch signalling may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In the present invention Notch signalling means specific signalling, meaning that the signal detected results substantially or at least predominantly from the Notch signalling pathway, and preferably from Notch/Notch ligand interaction, rather than any other significant interfering or competing cause, such as cytokine signalling. In one embodiment the term “Notch signalling” excludes cytokine signalling. The Notch signalling pathway is described in more detail below.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (eg chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

In one embodiment the Notch ligand which activates Notch may be expressed on a cell or cell membrane, suitably derived from a cell.

Candidate modulators may be synthetic compounds or natural isolated compounds. Various examples of such synthetic or natural modulators are listed below.

Candidate Modulators: Antagonists

Antagonists of Notch signalling will include any molecule which is capable of inhibiting Notch, the Notch signalling pathway or any one or more of the components of the Notch signalling pathway.

Candidate modulators for Notch signalling inhibition may be dominant negative versions of a compound capable of activating or transducing Notch signalling. Alternatively, the candidate modulator of Notch signalling will be capable of repressing a compound capable of activating or transducing Notch signalling. In a further alternative embodiment, the modulator will be an inhibitor of Notch signalling.

In a particular embodiment, the modulator will be capable of reducing or preventing Notch or Notch ligand expression. Such a modulator may be a nucleic acid sequence capable of reducing or preventing Notch or Notch ligand expression. Endogenous such modulators include nucleic acid sequences encoding a polypeptide selected from Toll-like receptor protein family, a cytokine such as IL-1 2, IFN-γ, TNF-α, or a growth factor such as a bone morphogenetic protein (BMP), a BMP receptor and activins. Candidate modulators will include derivatives, fragments, variants, mimetics, analogues and homologues of any of the above.

In a preferred embodiment, the modulator will be a polypeptide, or a polynucleotide encoding such a polypeptide, that decreases or interferes with the production of compounds that are capable of producing an increase in the expression of Notch ligand. Endogenous compounds of this type include Noggin, Chordin, Follistatin, Xnr3, fibroblast growth factors. Candidate modulators will include derivatives, fragments, variants, mimetics, analogues and homologues of any of the above.

Alternatively, the candidate modulator will be an antisense construct derived from a sense nucleotide sequence encoding a polypeptide selected from a Notch ligand and a polypeptide capable of up-regulating Notch ligand expression, such as Noggin, Chordin, Follistatin, Xnr3, fibroblast growth factors and derivatives, fragments, variants, mimetics, analogues and homologues thereof.

In another preferred embodiment the candidate modulator for Notch signalling inhibition will be a molecule which is capable of modulating Notch-Notch ligand interactions. A molecule may be considered to modulate Notch-Notch ligand interactions if it is capable of inhibiting the interaction of Notch with its ligands, preferably to an extent sufficient to provide therapeutic efficacy. In this embodiment the modulator may be a polypeptide, or a polynucleotide encoding such a polypeptide, selected from a Toll-like receptor, a cytokine such as IL-12, IFN-γ, TNF-α, or a growth factor such as a BMP, a BMP receptor and activins, derivatives, fragments, variants, mimetics, homologues and analogues thereof Preferably the modulator will decrease or interfere with the production of an agent that is capable of producing an increase in the expression of Notch ligand, such as Noggin, Chordin, Follistatin, Xnr3, fibroblast growth factors and derivatives, fragments, variants, mimetics homologues and analogues thereof.

Preferably when the modulator is a receptor or a nucleic acid sequence encoding a receptor, the receptor is activated. Thus, for example, when the modulator is a nucleic acid sequence, the receptor is constitutively active when expressed.

Modulators for Notch signalling inhibition also include downstream modulators of the Notch signalling pathway (such as Dsh, Numb and derivatives, fragments, variants, mimetics, homologues and analogues thereof), compounds that prevent expression of Notch target genes or induce expression of genes repressed by the Notch signalling pathway and dominant negative versions of Notch signalling transducer molecules (such as of NotchIC, Deltex and derivatives, fragments, variants, mimetics, homologues and analogues thereof). Proteins for Notch signalling inhibition will also include variants of the wild-type components of the Notch signalling pathway which have been modified in such a way that their presence blocks rather than transduces the signalling pathway. An example of such a modulator would be a Notch receptor which has been modified such that proteolytic cleavage of its intracellular domain is no longer possible.

Candidate Modulators: Agonists

Agonists of Notch signalling will include any molecule which is capable of up-regulating Notch, the Notch signalling pathway or any one or more of the components of the Notch signalling pathway. Candidate modulators for up-regulating the Notch signalling pathway include compounds capable of transducing or activating the Notch signalling pathway.

Modulators for Notch signalling transduction will include molecules which participate in signalling through Notch receptors including activation of Notch, the downstream events of the Notch signalling pathway, transcriptional regulation of downstream target genes and other non-transcriptional downstream events (e.g. post-translational modification of existing proteins). More particularly, such modulators will allow activation of target genes of the Notch signalling pathway.

According to one aspect of the present invention the modulator may be the Notch polypeptide or polynucleotide or a fragment, variant, derivative, mimetic or homologue thereof which retains the signalling transduction ability of Notch or an analogue of Notch which has the signalling transduction ability of Notch. By Notch, we mean Notch-1, Notch-2, Notch-3, Notch-4 and any other Notch homologues or analogues. Analogues of Notch include proteins from the Epstein Barr virus (EBV), such as EBNA2, BARF0 or LMP2A. In a particularly preferred embodiment the modulator may be the Notch intracellular domain (Notch IC) or a sub-fragment, variant, derivative, mimetic, analogue or homologue thereof.

Modulators for Notch signalling activation include molecules which are capable of activating Notch, the Notch signalling pathway or any one or more of the components of the Notch signalling pathway.

Such a modulator may be a dominant negative version of a Notch signalling repressor. In an alternative embodiment, the modulator will be capable of inhibiting a Notch signalling repressor. In a further alternative embodiment, the modulator for Notch signalling activation will be a positive activator of Notch signalling.

In a particular embodiment, the modulator will be capable of inducing or increasing Notch or Notch ligand expression. Such a molecule may be a nucleic acid sequence capable of inducing or increasing Notch or Notch ligand expression.

In one embodiment, the modulator will be capable of up-regulating expression of the endogenous genes encoding Notch or Notch ligands in target cells. In particular, the modulator may be an immunosuppressive cytokine capable of up-regulating the expression of endogenous Notch or Notch ligands in target cells, or a polynucleotide which encodes such a cytokine. Immunosuppressive cytokines include IL-4, IL-10, IL-13, TGF-β and FLT3 ligand. Candidate modulators will therefore further include fragments, derivatives, variants, mimetics, analogues and homologues of any of the above.

Endogenous agonists include Noggin, Chordin, Follistatin, Xnr3, fibroblast growth factors. Candidate modulators may therefore include derivatives, fragments, variants, mimetics, analogues and homologues thereof, or a polynucleotide encoding any one or more of the above.

In another embodiment, the modulator may be a Notch ligand, or a polynucleotide encoding a Notch ligand. Notch ligands will typically be capable of binding to a Notch receptor polypeptide present in the membrane of a variety of mammalian cells, for example hemapoietic stem cells. Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and U.S. Pat. No. 6,121,045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-i (Genbank Accession No. U73936—Homo sapiens) and Jagged-2 (Genbank Accession No. AF029778—Homo sapiens), and LAG-2. Homology between family members is extensive.

In a preferred embodiment, the modulator will be a constitutively active Notch receptor or Notch intracellular domain, or a polynucleotide encoding such a receptor or intracellular domain.

In an alternative embodiment, the modulator of Notch signalling will act downstream of the Notch receptor. Thus, for example, the activator of Notch signalling may be a constitutively active Deltex polypeptide or a polynucleotide encoding such a polypeptide. Other endogenous downstream components of the Notch signalling pathway include Deltex-1, Deltex-2, Deltex-3, Suppressor of Deltex (SuDx), Nunb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes. Candidate modulators of use in the present invention will therefore include constitutively active forms of any of the above, analogues, homologues, derivatives, variants, mimetics and fragments thereof.

Modulators for Notch signalling activation may also include any polypeptides expressed as a result of Notch activation and any polypeptides involved in the expression of such polypeptides, or polynucleotides encoding for such polypeptides.

Activation of Notch signalling may also be achieved by repressing inhibitors of the Notch signalling pathway. As such, candidate modulators will include molecules capable of repressing any Notch signalling inhibitors. Preferably the molecule will be a polypeptide, or a polynucleotide encoding such a polypeptide, that decreases or interferes with the production or activity of compounds that are capable of producing an decrease in the expression or activity of Notch, Notch ligands, or any downstream components of the Notch signalling pathway. In a preferred embodiment, the modulators will be capable of repressing polypeptides of the Toll-like receptor protein family, cytokines such as IL-12, IFN-γ, TNF-α, and growth factors such as the bone morphogenetic protein (BMP), BMP receptors and activins.

Polypeptides and Polynucleotides for Notch Signalling Transduction

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as disulphide-linked heterodimeric molecules consisting of an extracellular domain containing up to 36 epidermal growth factor (EGF)-like repeats [Notch ½=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

S3 processing occurs only in response to binding of Notch ligands Delta or Serrate/Jagged. The post-translational modification of the nascent Notch receptor in the Golgi (Munro; Ju) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin; Hicks). Although Drosophila has a single Fringe gene, vertebrates are known to express multiple genes (Radical, Manic and Lunatic Fringes) (Irvine).

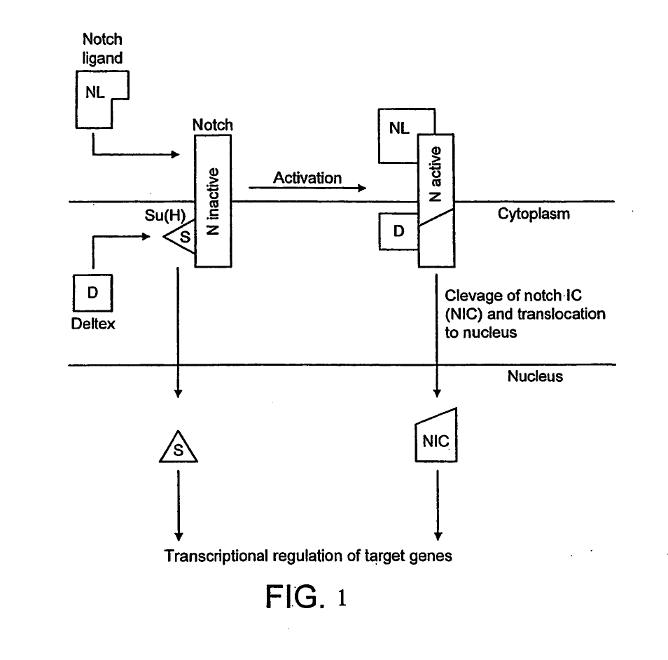

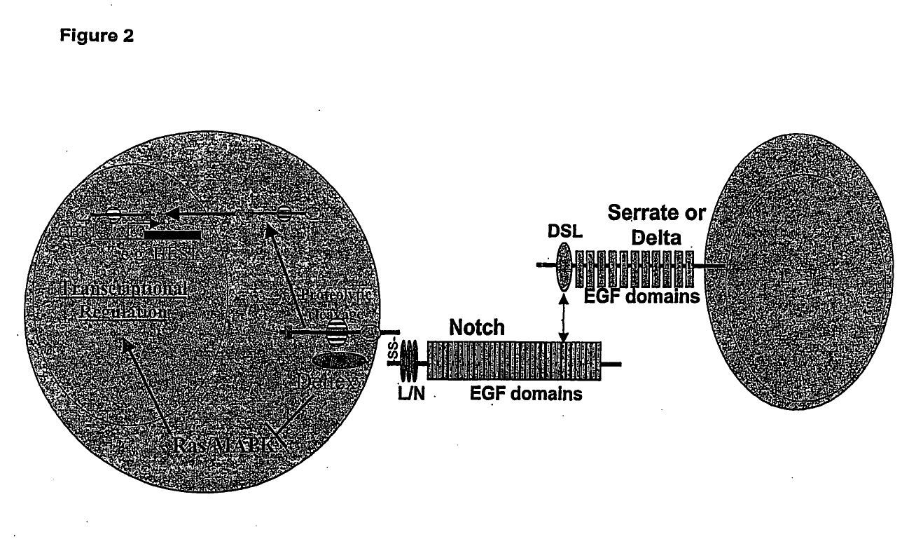

Signal transduction from the Notch receptor can occur via two different pathways (FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltx. Unlike CBF1, Deltex does not move to the nucleus following Notch activation but instead can interact with Grb2 and modulate the Ras-JNK signalling pathway.

Thus, signal transduction from the Notch receptor can occur via two different pathways both of which are illustrated in FIG. 1. Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. EL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. G11041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. G11783344.

Dlx-1 (distalless-1). (McGuiness) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

Polypeptides and Polynucleotides for Notch Signalling Activation

Examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive. For example, human Jagged-2 has 40.6% identity and 58.7% similarity to Serrate.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequence of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example http://www.ncbi.nlm.nih.gov and Ausubel et al., Current Protocols in Molecular Biology (1995), John Wiley & Sons, Inc.)

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Polypeptide substances may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz) and other ADAMALYSIN gene family members.

Polypeptides and Polynucleotides for Notch Signalling Inhibition

Suitable nucleic acid sequences may include anti-sense constructs, for example nucleic acid sequences encoding antisense Notch ligand constructs as well as antisense constructs designed to reduce or inhibit the expression of upregulators of Notch ligand expression (see above). The antisense nucleic acid may be an oligonucleotide such as a synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Preferably, the nucleic acid sequence for use in the present invention is capable of inhibiting Serrate and Delta, preferably Serrate 1 and Serrate 2 as well as Delta 1, Delta 3 and Delta 4 expression in APCs such as dendritic cells. In particular, the nucleic acid sequence may be capable of inhibiting Serrate expression but not Delta expression, or Delta but not Serrate expression in APCs or T cells. Alternatively, the nucleic acid sequence for use in the present invention is capable of inhibiting Delta expression in T cells such as CD4⁺ helper T cells or other cells of the immune system that express Delta (for example in response to stimulation of cell surface receptors). In particular, the nucleic acid sequence may be capable of inhibiting Delta expression but not Serrate expression in T cells. In a particularly preferred embodiment, the nucleic acid sequence is capable of inhibiting Notch ligand expression in both T cells and APC, for example Serrate expression in APCs and Delta expression in T cells.

Molecules for inhibition of Notch signalling will also include polypeptides, or polynucleotides which encode therefore, capable of modifying Notch-protein expression or presentation on the cell membrane or signalling pathways. Molecules that reduce or interfere with its presentation as a fully functional cell membrane protein may include MMP inhibitors such as hydroxymate-based inhibitors.

Other substances which may be used to reduce interaction between Notch and Notch ligands are exogenous Notch or Notch ligands or functional derivatives thereof. Such Notch ligand derivatives would preferably have the DSL domain at the N-terminus and up to about 14 or more, for example between about 3 to 8 EGF-like repeats on the extracellular surface. A peptide corresponding to the Delta/Serrate/LAG-2 domain of hJagged1 and supernatants from COS cells expressing a soluble form of the extracellular portion of hJagged1 was found to mimic the effect of Jagged1 in inhibiting Notch1 (Li).

Other Notch signalling pathway antagonists include antibodies which inhibit interactions between components of the Notch signalling pathway, e.g. antibodies to Notch ligands.

Whether a substance can be used for modulating Notch-Notch ligand expression may be determined using suitable screening assays.

Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. For example, an increase in levels of Deltex, Hes-1 and/or IL-10 mRNA may, for instance, indicate induced anergy while an increase in levels of IFN-γ mRNA, or in the levels of mRNA encoding cytokines such as IL-2, IL-5 and IL-13, may indicate improved responsiveness.

Many compounds identified according to the present invention may be lead compounds useful for drug development. Useful lead compounds include antibodies and peptides, and including intracellular antibodies expressed within the cell in a gene therapy context, which may be used as models for the development of peptide or low molecular weight therapeutics. In a preferred aspect of the invention, lead compounds and the Notch receptor or Notch ligand or other target peptides may be co-crystallised in order to facilitate the design of suitable low molecular weight compounds which mimic the interaction observed with the lead compound.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to and/or modulating components of the Notch signalling pathway. These include the use of phage display systems for expressing signalling proteins, and using a culture of transfected E. coli or other microorganism to produce the proteins for studies of potential binding and/or modulating compounds (see, for example, G. Cesarini, FEBS Letters, 307(1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Notch Ligands

As discussed above, Notch ligands comprise a number of distinctive domains. Some predicted/potential domain locations for various naturally occurring human Notch ligands (based on amino acid numbering in the precursor proteins) are shown below:

| | | |
|---|---|---|
| Component | Amino acids | Proposed function/domain |

Human Delta 1

| | | |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| | | |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| | | |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| | | |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| | | |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence:

Cys Xaa Xaa Xaa Xaa Xaa Xaa Xaa Xaa Cys Xaa Xaa Xaa Cys Xaa Xaa
Xaa Xaa Xaa Xaa Xaa Xaa Xaa Xaa Xaa Cys Xaa Xaa Xaa Xaa Xaa Xaa
Xaa Cys Xaa Xaa Xaa Xaa Xaa Xaa Xaa Xaa Cys

Preferably the DSL domain may include most or all of the following consensus amino acid sequence:

Cys Xaa Xaa Xaa ARO ARO Xaa Xaa Xaa Cys Xaa Xaa Xaa Cys BAS NOP
BAS ACM ACM Xaa ARO NOP ARO Xaa Xaa Cys Xaa Xaa Xaa NOP Xaa Xaa
Xaa Cys Xaa Xaa NOP ARO Xaa NOP Xaa Xaa Cys

wherein:
ARO is an aromatic amino acid residue, such as tyrosine, phenylalanine, tryptophan or histidine;
NOP is a non-polar amino acid residue such as glycine, alanine, proline, leucine, isoleucine or valine;
BAS is a basic amino acid residue such as arginine or lysine; and
ACM is an acid or amide amino acid residue such as aspartic acid, glutamic acid, asparagine or glutamine.

Preferably the DSL domain may include most or all of the following consensus amino acid sequence:

Cys Xaa Xaa Xaa Tyr Tyr Xaa Xaa Xaa Cys Xaa Xaa Xaa Cys Arg Pro
Arg Asx Asp Xaa Phe Gly His Xaa Xaa Cys Xaa Xaa Xaa Gly Xaa Xaa
Xaa Cys Xaa Xaa Gly Trp Xaa Gly Xaa Xaa Cys

(wherein Xaa may be any amino acid and Asx is either aspartic acid or asparagine).

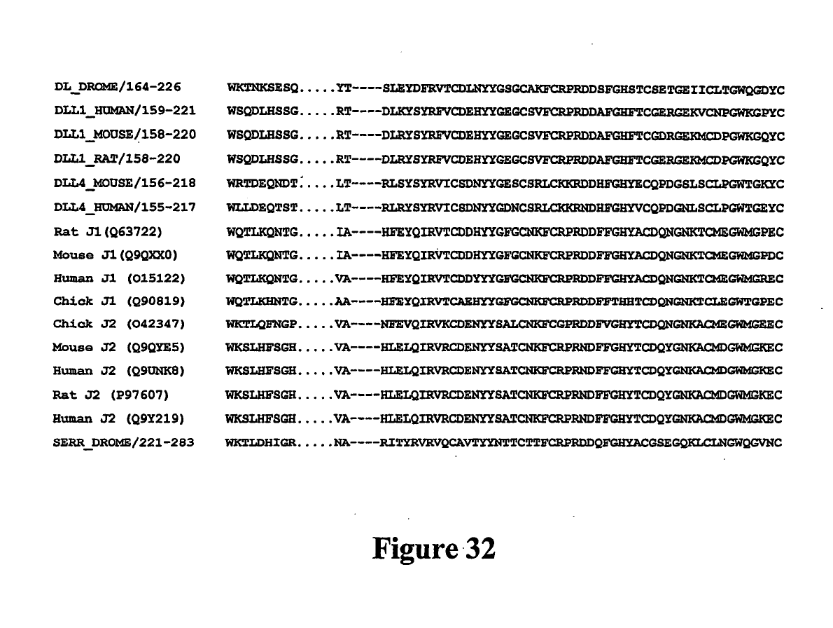

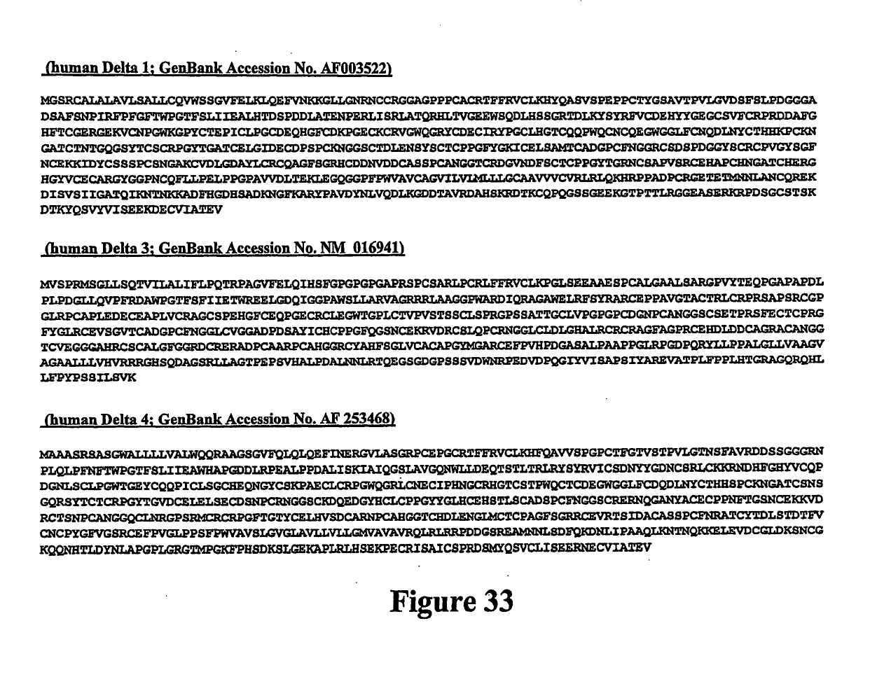

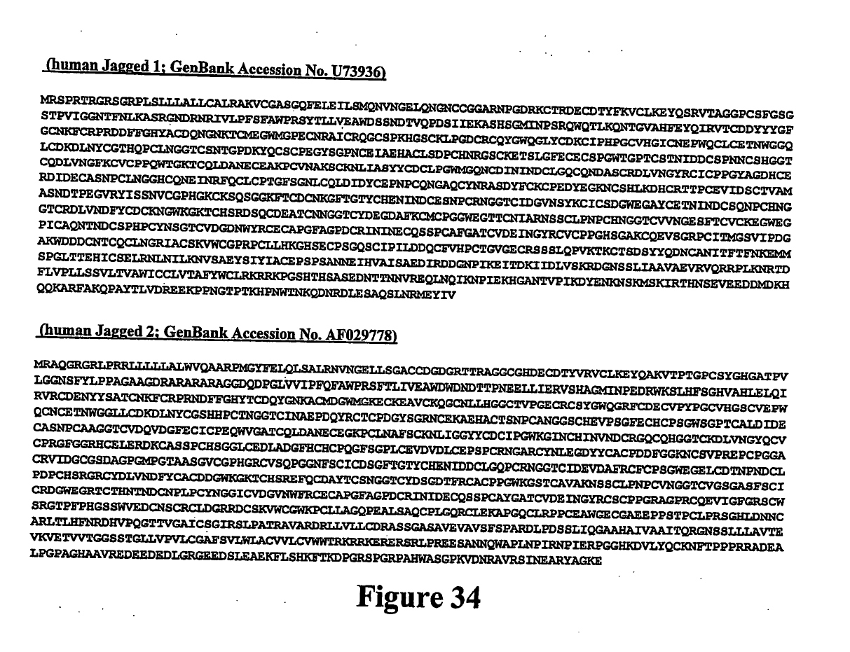

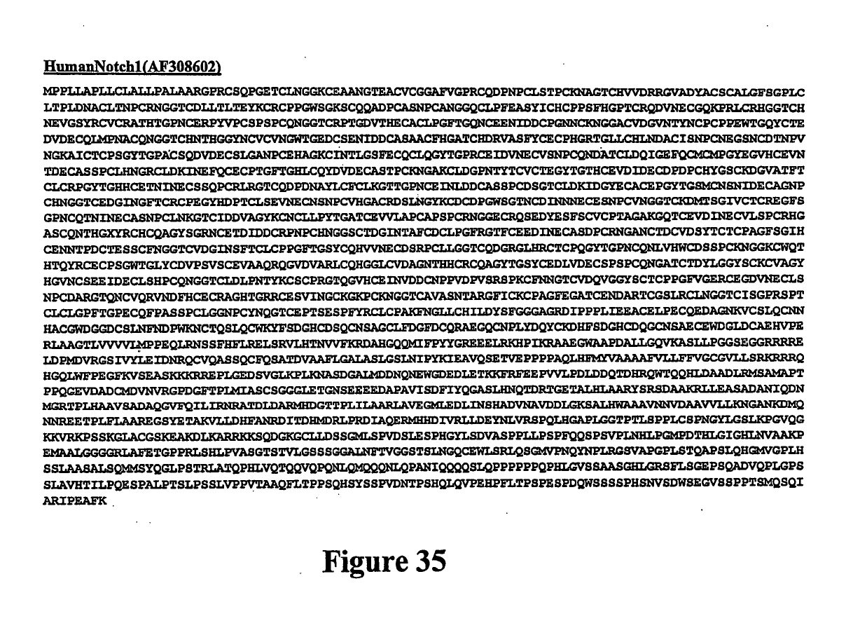

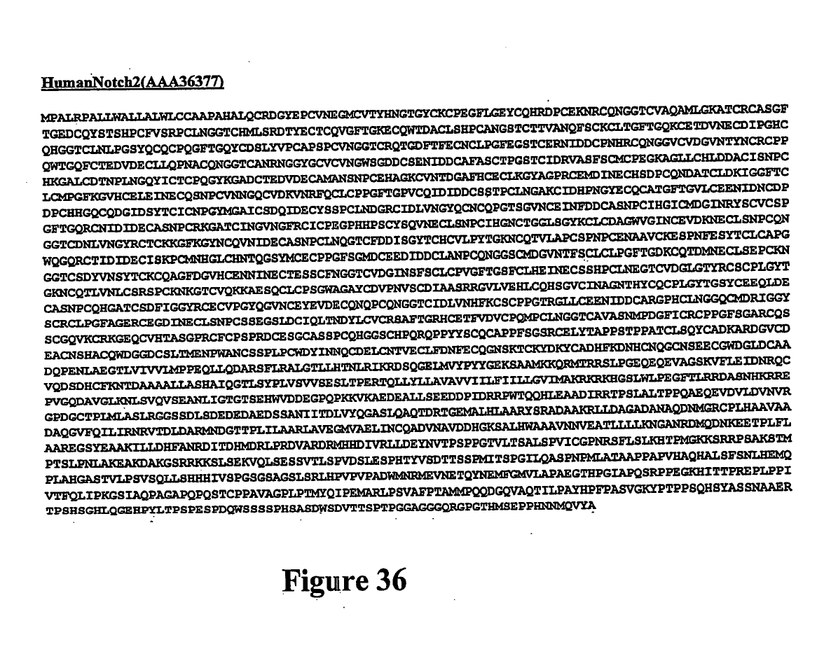

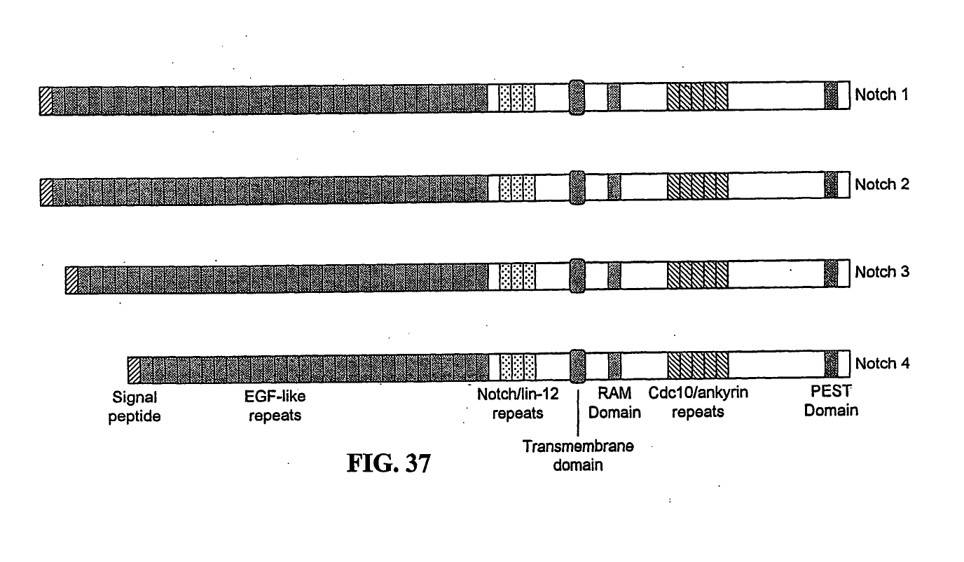

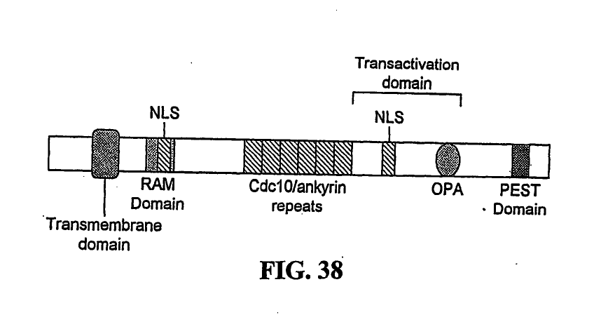

An alignment of DSL domains from Notch ligands from various sources is shown in FIG. 32.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE the EGF domain typically includes six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of the EGF-like domain: 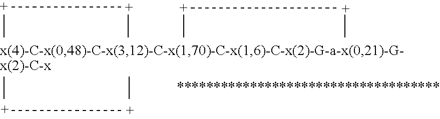
wherein:
‘C’: conserved cysteine involved in a disulfide bond.
‘G’: often conserved glycine
‘a’: often conserved aromatic amino acid
‘*’: position of both patterns.
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity. Suitable parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

Polypeptide Sequences

As used herein, the term “polypeptide” is synonymous with the term “amino acid sequence” and/or the term “protein”. In some instances, the term “polypeptide” is synonymous with the term “peptide”.

“Peptide” usually refers to a short amino acid sequence that is 10 to 40 amino acids long, preferably 10 to 35 amino acids.

The polypeptide sequence may be prepared and isolated from a suitable source, or it may be made synthetically or it may be prepared by use of recombinant DNA techniques.

Polynucleotide Sequences

As used herein, the term “polynucleotide sequence” is synonymous with the term “polynucleotide” and/or the term “nucleotide sequence”.

The polynucleotide sequence may be DNA or RNA of genomic or synthetic or of recombinant origin. They may also be cloned by standard techniques. The polynucleotide sequence may be double-stranded or single-stranded whether representing the sense or antisense strand or combinations thereof.

“Polynucleotide” refers to a polymeric form of nucleotides of at least 10 bases in length and up to 1,000 bases or even more. Longer polynucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

The polynucleotide sequence may comprise, for example, a protein-encoding domain, an antisense sequence or a functional motif such as a protein-binding domain and includes variants, derivatives, analogues and fragments thereof. The term also refers to polypeptides encoded by the nucleotide sequence.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transfection, microinjection, cationic lipid-mediated transfection, electroporation, transduction, scrape loading, ballistic introduction and infection. It will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Variants, Derivatives, Analogues, Homologues and Fragments

In addition to the specific polypeptide and polynucleotide sequences mentioned herein, the present invention also encompasses the use of variants, derivatives, analogues, homologues, mimetics and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides, includes any polypeptide or polynucleotide which retains at least one of the functions of the endogenous polypeptide or polynucleotide but generally has a different evolutionary origin thereto.

The term “mimetic” as used herein, in relation to polypeptides or polynucleotides, refers to a chemical compound that possesses at least one of the endogenous functions of the polypeptide or polynucleotide which it mimics.

Typically, amino acid substitutions may be made, for example from 1, 2 or 3 to 10 or 20 substitutions provided that the modified sequence retains the required transport activity or ability to modulate Notch signalling. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | 3- | | |
|---|---|---|---|
| Symbol | letter | Meaning | Codons |
| A | Ala | Alanine | GCT, GCC, GCA, GCG |
| B | Asp, | Aspartic, | GAT, GAC, AAT, AAC |
| | Asn | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, CTA, |
| | | | CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, AGA, |
| | | | AGG |
| S | Ser | Serine | TCT, TCC, TCA, TCG, AGT, |
| | | | AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, | Glutamic, | GAA, GAG, CAA, CAG |
| | Gln | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleodtide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gene expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (Devereux). Examples of other software than can perform sequence comparisons include, but are not limited to, the BLAST package, FASTA (Atschul) and the GENEWORKS suite of comparison tools. Both BLAST and FASTA are available for offline and online searching. However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

In a first step of the method of the present invention, any one or more of the above candidate modulators is brought into contact with a cell of the immune system. Cells of the immune system of use in the present invention are described below.

By Notch, we mean Notch-1, Notch-2, Notch-3 or Notch-4 and any other Notch homologues or analogues. The term “Notch IC” includes the full intracellular domain of Notch or an active portion of this domain. For example, the sequence may be a sequence comprising or coding for at least amino acids 1848 to 2202 of human Notch1 or a sequence having at least 70%, preferably at least 75%, preferably at least 80%, preferably at least 85%, preferably at least 90%, preferably at least 95% amino acid sequence similarity or identity with this sequence. The sequence may also suitably be derived from human Notch2, Notch3 or Notch4. Suitably the Notch sequence comprises at least a Notch Ankyrin repeat domain and optionally a Notch LNR domain, Notch RAM domain, Notch OPA domain and/or Notch PEST sequence.

Cells of the Immune System

Cells of use in the present invention are cells of the immune system capable of transducing the Notch signalling pathway.

Most preferably the cells of use in the present invention are T-cells. These include, but are not limited to, CD4⁺ and CD8⁺ mature T cells, immature T cells of peripheral or thymic origin and NK-T cells.

Alternatively, the cells will be antigen-presenting cells (APCs). APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, constitutively expressing or activated to express a MHC Class II molecules on their surfaces. Precursors of APCs include CD34⁺ cells, monocytes, fibroblasts and endothelial cells. The APCs or precursors may be modified by the culture conditions or may be genetically modified, for instance-by transfection of one or more genes.

The T cells or APCs may be isolated from a patient, or from a donor individual or another individual. The cells are preferably mammalian cells such as human or mouse cells. Preferably the cells are of human origin. The APC or precursor APC may be provided by a cell proliferating in culture such as an established cell line or a primary cell culture. Examples include hybridoma cell lines, L-cells and human fibroblasts such as MRC-5. Preferred cell lines for use in the present invention include Jurkat, H9, CEM and EL4 T-cells; long-term T-cell clones such as human HA1.7 or mouse D10 cells; T-cell hybridomas such as DO11.10 cells; macrophage-like cells such as U937 or THP1 cells; B-cell lines such as EBV-transformed cells such as Raji, A20 and M1 cells.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba et al), or from bone marrow, non-adherent CD34⁺ cells can be treated with GM-CSF and TNF-α (Caux et al). DCs can also be routinely prepared from the peripheral blood of human volunteers, similarly to the method of Sallusto and Lanzavecchia J Exp Med (1994) 179(4) 1109-18 using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin et al). Culture conditions may include other cytokines such as GM-CSF or IL-4 for the maintenance and, or activity of the dendritic cells or other antigen presenting cells.

T cells and B cells for use in the invention are preferably obtained from cell lines such as lymphoma or leukemia cell lines, T cell hybridomas or B cell hybridomas but may also be isolated from an individual suffering from a disease of the immune system or a recipient for a transplant operation or from a related or unrelated donor individual. T cells and B cells may be obtained from blood or another source (such as lymph nodes, spleen, or bone marrow) and may be enriched or purified by standard procedures. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used.

Candidate modulators of use in the present invention are brought into contact with a cell of the immune system as described above. In a further step, modulation of Notch signalling by a candidate modulator is detected. Assays for detecting modulation of Notch signalling will be described below. Many of these assays will involve monitoring the expression of a “target gene”.

Target Genes

The target genes of use in the present invention may be endogenous target genes (i.e. endogenous target genes of the Notch signalling pathway) or synthetic reporter genes.

Endogenous Target Genes

Endogenous target genes of the Notch signalling pathway include Deltex, genes of the Hes family (Hes-1 in particular), Enhancer of Split [E(spl)] complex genes, Il-10, CD-23, Dlx-1, CTLA4, CD-4, Dll-1, Numb, Mastermind and Dsh. Although all genes the expression of which is modulated by Notch activation may be used for the purpose of the present invention, preferred endogenous target genes are described below.

Deltex, an intracellular docking protein, replaces Su(H) as it leaves its site of interaction with the intracellular tail of Notch, as shown in FIG. 1. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by,Notch activation, and Hes-3. Expression of Hes-1 is upregulated as a result of Notch activation. The sequence of human Hes-1 can be found in GenBank Accession Nos. AK000415 and AF264785.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation. IL-10 (interleukin-10) is a factor produced by Th2 helper T-cells. It is a co-regulator of mast cell growth and shows extensive homology with the Epstein-Barr bcrfi gene. Although it is not known to be a direct downstream target of the Notch signalling pathway, its expression has been found to be strongly upregulated coincident with Notch activation. The mRNA sequence of IL-10 may be found in GenBank ref. No. G11041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. Although it is not thought to be a direct downstream target of the Notch signalling pathway, its expression has been found to be strongly upregulated coincident with Notch activation. The sequence for CD-23 may be found in GenBank ref. No. G11783344.

Dlx-1 (distalless-1) expression is downregulated as a result of Notch activation. Sequences for Dlx genes may be found in GenBank Accession Nos. U51000-3.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF1 response elements and its expression is upregulated as a result of Notch activation. The sequence of CTLA4 can be found in GenBank Accession No. L15006.

CD-4 expression is downregulated as a result of Notch activation. A sequence for the CD-4 antigen may be found in GenBank Accession No. XM006966.

Other useful target genes include genes associated with anergy, such as (with associated GenBank Accession Nos):

GRG4 (groucho-related protein U61363), Ikaros (L03547), Jumonji (D31967), Caspase 3 (U54803), SOCS2 (U88327), Traf5 (D78141), RPTPσ (D28530), RPTPκ (L10106), PTP-1B (U24700), AGKα (AA066032), LDHAα (Y00309), Pgam1 (phosphoglycerate mutase—AA161799), GBP-3 (guanylate binding protein 3—U44731), RGS-2 (G-protein signaling regulator 2—U67187), Rab10 (AA119194), CD98 (U25708), 4-1BB-L (L15435), FasL (U06948), Hif-1 (Hypoxia inducible factor 1 AF003695), SATB1 (nuclear matrix attachment DNA-binding protein—U05252), Elf-1 (U19617), NFIL3 (U83148), RNF19 (also called GEG-154—X71642), Mlp (Markcks-like protein—AA245242), Lad/TSAd (p561ck-associated adapter protein—ET62419), ZAP-70 (U04379), Serpin 1b (AA125310), Cytostatin C (M59470), glutamate dehydrogenase (X57024), CD3 epsilon (J02990), cation-dependent mannose-6-phosphate receptor (X64068), gamma-aminobutyric acid receptor-associated protein-like protein-1 (Z31137), tetracycline transporter-like protein (D88315), MCSF (M21952), Calcyclin (M37761), Heme oxygenase 2a (Z31202) and Osp94 (osmotic stress protein 94-U23921).

Preferably the target/reporter gene is not IL-2 or NF-AT.

Synthetic Reporter Genes

In an alternative embodiment of the present invention, the target gene is a reporter gene. In a preferred embodiment, the reporter gene is under the transcriptional control of a promoter region or responder element(s) sensitive to Notch signalling.

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Other protocols include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes may even be used. These and other assays are described, among other places, in Hampton R et al (1990, Serological Methods, A Laboratory Manual, APS Press, St Paul Minn.) and Maddox DE et al.

One skilled in the art will recognize that the identity of the specific reporter gene can, of course, vary. Examples of reporter genes that have been used in the art include, but are not limited to, genes encoding an enzymatic activity such as chloramphenicol acetyltransferase (CAT) gene, Green Fluorescent Protein (GFP), luciferase (luc), β-galactosidase, invertase, horseradish peroxidase, glucuronidase, exo-glucanase, glucoamylase or alkaline phosphatase. Alternatively, the reporter gene may comprise a radiolabel or a fluorescent label such as FITC, rhodamine, lanthanide phosphors, or a green fluorescent fusion protein (See for example Stauber et al). Alternatively, the reporter may comprise a predetermined polypeptide epitope which can be recognized by a secondary reporter such as leucine zipper pair sequences, binding sites for secondary antibodies, metal binding domains, or epitope tags. One skilled in the art will appreciate that the specific reporter gene or genes utilized in the methods disclosed herein may vary and may also depend on the specific model system utilized, and the methods disclosed herein are not limited to any specific reporter gene or genes.

By way of further examples, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

The reporter gene used in the method of the present invention is under the transcriptional control of at least one Notch signalling sensitive promoter region and/or responder element. Promoter regions and/or responder elements sensitive to Notch signalling include the regulatory elements of endogenous Notch target genes such as the HES promoters, Deltex promoter, Notch and Notch ligand promoters, IL-10 promoters. Regulatory elements of use in the present invention also include single or multimerized CBF1 sites, CTLA4 promoters and AIRE promoters. The regulatory elements are positioned such that activation of the Notch signalling pathway results in increased expression of the reporter gene.

One or more copies of the reporter gene can be inserted into the host cell by methods known in the art. The term “host cell”—in relation to the present invention includes any cell that could comprise the target for the agent of the present invention. Polynucleotides may be introduced into prokaryotic cells or eukaryotic cells, for example yeast, insect or mammalian cells. Preferably, the host cell will be a cell of the immune system as described above.

Polynucleotides of the invention may be introduced into suitable host cells using a variety of techniques known in the art, such as transfection, transformation and electroporation. Where polynucleotides of the invention are to be administered to animals, several techniques are known in the art, for example infection with recombinant viral vectors such as retroviruses, herpes simplex viruses and adenoviruses, direct injection of nucleic acids and biolistic transformation.

In the present invention, the host cells will preferably be mammalian cells and the polypeptides will be expressed either intracellularly, on the cell membranes or secreted in a culture media if preceded by an appropriate leader sequence.

Expression of the target genes (whether endogenous or synthetic reporter genes) may be dependent on Notch signalling alone or on Notch signalling and one or more further stimulatory signals.

Stimulatory Signals

Expression or repression of the target genes (endogenous or reporter genes) of use in the present invention is dependent on Notch signalling. In a preferred embodiment, expression or repression of the target genes will additionally be depend on a second immune cell specific stimulus, with or without an accessory signal (or “costimulus”).

In one embodiment, the second stimulus will result from activation of an immune cell receptor. Examples of immune cell receptors include T cell receptors (TCR), B cell receptors (BCR) and Toll-like receptors (TLR). Examples of molecules capable of triggering a TCR or BCR signal include specific antigens for the receptors, superantigens such as TSS1, SEA, SEB, SEC, SED and SEE, antibodies to the TCR αβ chains including Fab, F(ab)2 fragments, phage displayed peptides and ScFV or antibodies to CD3 proteins including ζ and ε chains, anti-CD28 antibodies, anti-BCR antibodies, LPS and other bacterial products, cell receptors involved in phagocytosis such as Fc receptors, complement receptors, mannose receptors and other scavenger receptors, receptors involved in clearance of apoptotic cells such as CD36 and αvβ5, dendritic cell receptors such as DEC205 and DC-light, and activators of TCR and/or BCR signalling pathways such as PMA, ionomycin or kinase inhibitors. These molecules may be used alone or in combination and may be presented on an antigen presenting cell.

In accordance with one embodiment of the present invention there is provided a method for detecting modulators of Notch signalling comprising the steps of:
(a) activating a cell of the immune system;
(b) contacting the cell with a candidate modulator;
(c) monitoring Notch signalling;
   (wherein steps (a), (b) and (c) can be carried out in any order); and
(d) determining whether the candidate modulator modulates Notch signalling.

Preferably the activator is an anti-CD3 antibody or an anti-CD28 antibody. In more detail, T cell activation involves multiple intracellular signaling events originating from the cell surface TCR/CD3 complex. Cross-linking of the TCR/CD3 complex by anti-CD3 antibodies induces T cell activation, leading to the production of cytokines such as IL-2. IL-2 binds to its high affinity receptor to promote cell proliferation. Additionally co-stimulatory surface molecules such as CD28 have been shown to provide accessory signals in T cell activation, enhancing IL-2 production, e.g. when combined with an anti-CD3 antibody. CD28 is an antigen expressed on the surface of T cells, and is also responsible for activation of T cells.

Accessory or costimulatory signals of immune cell receptor signalling include B7 proteins such as B7.1-CD80, B7.2-CD86, B7H1, B7H2, B7H3, B7RP1, B7RP2, CTLA4, ICOS, CD2, CD24, CD27, CD28, CD30, CD34, CD38, CD40, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, TRANCE/RANK ligands, Fas ligand, MHC class II, DEC205-CD205, CD204-Scavenger receptor, CD14, CD206 (mannose receptor), Toll-like receptors (TLRs), such as TLR 1-9, CD207 (Langerin), CD209 (DC-SIGN), FCγ receptor 2 (CD32), CD64 (FC-γ receptor 1), CD68, CD83, CD33, CD54, BDCA-2, BDCA-3, BDCA-4, chemokine receptors, cytokines, growth factors and growth factor receptor agonists, and variants, derivatives, analogues and fragments thereof.

In one embodiment, the second stimulus will be a costimulus. In an alternative embodiment, expression of the target genes will depend on three separate stimuli: Notch signalling, immune cell signalling and a costimulus, all of which are described above. The signals may be delivered all at once or may be phased over a defined period (possibly separated by hours or even days). Preferably, the signals will be delivered substantially simultaneously.

Immune Cell Activation

Immune cell activation may be monitored by any suitable method known to those skilled in the art. For example, cytotoxic activity may be monitored. Natural killer (NK) cells will demonstrate enhanced cytotoxic activity within 4 hours after activation. This cytotoxic activity is maximal after 18 hours.

Once activated, leukocytes express a variety of new cell surface antigens. NK cells, for example, will express transferrin receptor, HLA-DR and the CD25 IL-2 receptor after activation. Activation may therefore be assayed by monitoring expression of these antigens.

Hara et al. Human T Cell Activation: III, Rapid Induction of a Phosphorylated 28 kD/32 kD Disulfidelinked Early Activation Antigen (EA-1) by 12-0-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T cells within 18 hours and continue to appear as late as 48 hours after activation.

These antigens may be useful in detecting leukocyte activation. Additionally, leukocyte activation may be monitored as described in EP O 325 489 which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognizes a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T cells correlates with stimulation of the T cell antigen receptor complex, Anti-Leu 23 is useful in monitoring the activation or stimulation of leukocytes.

Further details of techniques for the monitoring of immune cell activation may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Suitably the immune cell is activated with a calcium signalling agent (such as a calcium ionophore, such as ionomycin) and/or an activator of a protein kinase (eg Protein Kinase C or MAP Kinase), such as phorbol myristate acetate (PMA). Alternatively, for example, a lectin such as phytohemagglutinin (PHA) may also be used to activate T cells (Nowell, P. C. (1990) Cancer Res. 20:462-466). Alternatively, for example, an antibody such as an anti-CD3, anti-T-cell Receptor antibody (anti-TCR antibody) and/or an anti-CD28 antibody may be used. A CD28 ligand, such as a protein comprising the co-activating domain of the B-cell antigen B7, may also be used.

Where a calcium ionophore such as ionomycin is used as activator, this may be used in concentrations of less than about 5 μg/ml, preferably less than about 1000 ng/ml, preferably less than about 250 ng/ml, preferably less than about 200 ng/ml, preferably less than about 100 ng/ml. Thus, for example, the concentration may range from about 0.01 ng/ml to about 5μg/ml, preferably from about 0.1 ng/ml to about 1000 ng/ml, suitably from about 0.1 ng/ml to about 250 ng/ml, preferably from about 1 ng/ml to about 200 ng/ml.

Where a calcium ionophore such as ionomycin is used as activator, this may be used in concentrations of less than about 10 μM, preferably less than about 5 μM, preferably less than about 2 μM, preferably less than about 0.5 μM, preferably less than about 0.1 μM. Suitably, for example, the ionophore is used in a range of from about 0.001 to 10 μM, for example about 0.01 to 0.5 μM.

A protein kinase activator may be used to activate the cells either in addition to or instead of a calcium ionophore. Suitably the kinase activator may be a MAP kinase activator (such as a member of one or more of the MAPKKK, MAPKK, MAPK families and their associated phosphatases, for example activators of the p38, Erk and Jnk pathways) or a protein kinase C activator (such as a phorbol ester, such as for example PMA or TPA).

Where a protein kinase activator is used, this may be used in concentrations of less than about 50 nM, preferably less than about 20 nM, preferably less than about 10 nM, preferably less than about 1 nM, preferably less than about 0.1 nM. Suitably, for example, the ionophore is used in a range of from about 0.001 to 10 nM, for example about 0.01 to 0.5 nM.

Preferably the immune cell is activated such as to permit at least 30% optimal, preferably at least 50% optimal, preferably at least 70% optimal, preferably at least 80% optimal, preferably at least 90% optimal, preferably at least 95% optimal levels of Notch or immune signalling. By “optimal” is meant the level of activation which maximises the response (as measured, for example, by reporter output) in the system used. By x% optimal is meant a level of activation which gives at least x% of the optimal response in the system used.

In some cases it may be desirable to operate a screen with optimal immune cell activation (for example to more readily identify inhibitors of Notch signalling) whilst in other cases it may be desirable to operate the screen with sub-optimal immune cell activation (for example to more readily identify activators of Notch signalling).

Likewise with Notch signalling activation, in some cases it may be desirable to operate a screen with optimal Notch activation (for example to more readily identify inhibitors of Notch signalling) whilst in other cases it may be desirable to operate the screen with sub-optimal Notch activation (for example to more readily identify activators of Notch signalling).

Preferably the Notch signalling activation is such as to permit at least 30% optimal, preferably at least 50% optimal, preferably at least 70% optimal, preferably at least 80% optimal, preferably at least 90% optimal, preferably at least 95% optimal levels of Notch or immune signalling. By “optimal” is meant the level of activation which maximises the response (as measured, for example, by reporter output) in the system used. By x% optimal is meant a level of activation which gives at least x% of the optimal response in the system used.

Notch Activation

Notch signalling may be activated in the immune cell in various ways. For example, the cell may already express Notch, in which case Notch signalling may be activated by activating Notch with, for example, a Notch ligand or an active portion thereof.

If the cell does not naturally express Notch, or it is desired to increase the expression (and therefore the signal), the cell may be transfected with Notch and Notch signalling may be activated with, for example, a Notch ligand or an active portion thereof.

Alternatively, the cell may be transfected with a constitutively active truncated form of Notch, in which case activation with Notch ligand etc is not necessary to establish Notch signalling. Such truncated forms of Notch are known, for example, from Lu et al, PNAS Vol 93, pp5663-5667 (May 1996) which is herein incorporated by reference. This document describes a truncated form of Notch wherein the extracellular domain is deleted (N1(δEC)).

Alternatively, the cell may be transfected with an expression vector expressing Notch intracellular domain (Notch IC) or an active part thereof, so that, once again, activation with Notch ligand etc is not necessary to establish Notch signalling.

Immune Signalling

The term “immune signalling” as used herein includes any signalling pathway for activation of cells of the immune system, preferably leukocytes, more preferably lymphocytes, and more preferably T-cells. Preferably immune signalling relates to a signalling pathway activated by activation of the T-cell receptor, B-cell receptor or a Toll-like receptor. Preferably immune signalling relates to any intracellular signalling pathway activated by activation of the T-cell receptor complex, where the term complex encompasses both protein chains of the T-cell receptor and CD3 molecules as well as membrane proteins providing costimulatory signals. These immune signalling pathways may be activated by physiological or engineered ligands for components of the membrane receptor complex, or other activators of proteins of the signalling pathway acting intracellularly in the cytoplasm and/or nucleus.

Lymphocyte activation is stimulated by clustering of their antigen receptors, by antigen/MHC complexes or antibodies to receptor components (for a general discussion see, for example, Immunobiology (4th Edition, 1999), by Janeway, Travers, Walport and Capra, published by Elsevier Science).

Signalling is initiated by the activation of protein tyrosine kinases, which associate with the receptor complex. Receptor clustering brings the enzymes into close proximity with each other and components of the receptor, leading to phosphorylation of tyrosine residues in both the kinases and cytoplasmic tails of the receptor protein chains. These phosphorylation events serve to provide interaction sites for other proteins involved in signalling and for activation of enzyme activities. Tyrosine phosphatases removing the phosho-groups from tyrosine residues are also involved in both activation events and in regulating the degree of activation.

Tyrosine kinases of the src family represent the first kinases involved in this receptor-mediated activation. For T-cells, lck and fyn play key initiating roles and serve to activate other tyrosine kinases such as ZAP-70. Similarly, for B-cells fyn, blk and lyn play similar roles, activating the kinase Syk. The receptor signalling chains of the T-cell receptor complex (CD3) or the B-cell receptor complex (Igα/Igβ) are tyrosine phosphorylated at tyrosine containing sequences called ITAMs (immunoreceptor tyrosine-based activation motifs), which have a canonical sequence of YXX[L/V]X₇₋₁₁YXX[L/V], where Y is tyrosine, L is leucine, V is valine and X represents any amino acid. These ITAMS serve as “docking sites” for other signalling proteins which bind via their SH2 phospho-tyrosine binding domains.

Several different classes of protein are recruited to these activated receptors. Phospholipase cγ (PLCγ) is recruited and activated to produce two key signalling mediators inositol trisphosphate (IP₃) and diacyl glycerol (DAG). IP₃ causes the release of calcium ions (Ca⁺⁺)into the cytoplasm from intracellular stores which in turn leads to the opening of calcium channels in the membrane that let more Ca⁺⁺ into the cell. This calcium influx serves to activate a number of calcium-binding proteins, including calmodulin and calcineurin, which together play a key role in transmitting signals to the nucleus to regulate gene transcription events, particularly activating members of the NFAT family of treanscription factors. DAG participates in the activation of different members of the protein kinase c (PKC) family of serine threonine kinases, some of which are also activated by Ca⁺⁺. PKC phosphorylates a number of other proteins in different signalling cascades, again mediating delivery of signals to the nucleus, especially through the activation of members of the NFκB family of transcription factors.

Small GTP-binding proteins (“small G-proteins”) are also involved in propagating signals from tyrosine kinase activated receptors. The best known of these is Ras. These small G-proteins exist in two different states depending on whether they are bound to GTP or GDP. The GTP bound form of Ras is the active form of the protein, whereas GDP-bound form is inactive. Ras itself has GTPase activity so can remove a phosphate and convert Ras back to the inactive form. Small G-proteins are normally found in their inactive state and activation requires a guanine nucleotide exchange factor (GEF) which helps exchange the GDP for GTP. In lymphocytes, Ras and other small G-proteins such as Rac are recruited to activated receptors by adapter proteins recruited to ITAMs; GEFs also bound to these adapters serve to activate these G-proteins.

There are many examples of adapter proteins involved in lymphocyte signal transduction. Two major ones for T-cells are LAT and Slp-76. LAT, activated by tyrosine phosphorylation, localizes to lipid rafts in membranes and binds directly or indirectly to a number of different adapter and signal propagation molecules, such as Grb2, SOS and Ras. For B-cells, BLNK may play a similar adapter role. Another adapter protein called Vav, which also has G-protein activity, plays an important role in B-cell signalling.

Activated G-proteins such as Ras are involved in activating several protein kinase cascades known as the mitogen activated protein kinase cascades (MAP kinase pathways). These cascades lead to phosphorylation and activation of different transcription factors and hence delivers signals to guide gene expression events in the nucleus. For example, the AP-1 family of transcription factors which are heterodimers of members of the fos and jun are important targets of these MAP kinase signalling pathways.

MAP kinase signalling cascades can be generically described as being activated by an activated small G-protein through action on the first kinase of the cascade which is called a MAP kinase kinase kinase (MAPKKK). This in turn phosphorylates and activates a MAP kinase kinase (MAPKK), which then phosphorylates and activates a MAP kinase (MAPK) protein, acting on two sites, a tyrosine and a threonine separated by a single amino acid. The double phosphorylated MAPK is then both enzymically active and able to migrate to the nucleus where it can phosphorylate transcription factors. Three major MAP kinase cascades have been defined, all of which are active in lymphocytes, which lead to the activation of the MAP kinases Erk (Erk1 and Erk2 particularly in lymphocytes), p38 and Jnk (JNK1 and JNK2 particularly in lymphocytes). Activators of Erk1 and Erk2 are called Mek1 and Mek2

Different co-receptors serve to enhance or modulate the antigen-receptor-mediated activation of lymphocytes. Examples being CD2, CD4, CD8 and CD45 in T-cells and CD19, CD21 and CD81 in B-cells. In addition co-stimulatory molecules also serve to enhance and modify the immune signalling. For example, CD28, CD40, OX40 and others can provide key signals that help determine both the quality and quantity of the cell's response. These molecules also activate signalling pathways which become integrated with signals emanating from the lymphocyte antigen receptor/co-receptor complexes, and include both tyrosine and serine/threonine kinases as well as small G-protein mediated cascades.

Different cytokines (e.g. IL-2, IL-4, IL-10, IFNg, IL-15 etc) also play important roles in regulating the responses of lymphocytes. Receptors for these cytokines use, among other pathways, a signal transduction pathway involving receptor activated kinases called Janus kinases (JAKS), which include JAK1,2 &3 and Tyk2. These phosphorylate a family of proteins called signal transducers and activators of transcription (STATS). This phosphorylation leads to homo- and heterodimerization of STATS mediated by their SH2 domains binding to STAT phosphotyrosine motifs. These dimers then translocate to the nucleus where they activate a variety of cytokine responsive genes. This activation pathway is regulated by a set of inhibitory proteins called SOCS proteins (e.g. SOCS1,2 & 3). Different cytokines activate different STATS. For example, the IL-4 receptor activates STAT6, which in turn plays a role in activating IL-4 responsive genes such as CD23. IL-12 activates STAT4 which plays a role in regulating IFNγ gene expression.

The integration of the different signals and their relative strengths determines the nature of the transcriptional response and the gene/protein expression profile and kinetics which in turn determines the overall nature of the response of the cell. For T-cells, for example, this will impact on the generation of effector and memory T-cell responses, different cytokine profiles and other effector functions or induce the cell to develop an unresponsive or anergic state. T-cells of different types will also have different quantitative and qualitative requirements for their different potential response states.

In APCs, different receptors can also transduce signals that regulate the activation and function of these cells. For example, Fc receptors, scavenger receptors and Toll-like receptors (TLRs) binding pathogenic material can provide signals that trigger responses in the APC that help those cells provide the right signals to the lymphocytes in order that they make the most effective response to clear the pathogen. TLRs are particularly important in this regard. TLRs (e.g TLR1, TLR2, TLR3 etc) are activated on binding different sets of molecules, often derived from pathogens (e.g. LPS, viral RNA, CpG motifs). This leads to the binding of an adapter protein called MyD88 to the cytoplasmic tail of the TLR, which leads to the activation of a kinase cascade culminating in activation of transcription factors, particularly of the NFκB family. These then serve to regulate the expression of genes encoding molecules that help activate and differentiate lymphocytes, particularly T-cells (e.g. surface proteins of the B7 family, cytokines such as IL-12 etc).

Assays

Assays for monitoring expression of the one or more target genes and other methods of detecting modulation of Notch signalling are described below.

The present invention preferably provides a cell-based assay for screening compounds for their ability to modulate Notch signalling. In one embodiment, the present invention provides an assay comprising the steps of:
(a) providing a culture of immune cells;
(b) optionally transfecting said cells with a reporter construct;
(c) optionally transfecting said cells with a Notch gene;
(d) exposing the cells to one or more compound(s) to be tested; and
(e) determining the difference in Notch signalling between cells exposed to the compound(s) to be tested and cells not so exposed.

The assay of the present invention is set up to detect either inhibition or enhancement of Notch signalling in cells of the immune system by candidate modulators. The method comprises mixing cells of the immune system, where necessary transformed or transfected, etc. with a synthetic reporter gene, in an appropriate buffer, with a sufficient amount of candidate modulator and monitoring Notch signalling. The modulators may be small molecules, proteins, antibodies or other ligands as described above. Amounts or activity of the target gene (also described above) will be measured for each compound tested using standard assay techniques and appropriate controls. Preferably the detected signal is compared with a reference signal and any modulation with respect to the reference signal measured.

The assay may also be run in the presence of a known antagonist of the Notch signalling pathway in order to identify compounds capable of rescuing the Notch signal.

Any one or more of appropriate targets—such as an amino acid sequence and/or nucleotide sequence—may be used for identifying a compound capable of modulating the Notch signalling pathway in cells of the immune system in any of a variety of drug screening techniques. The target employed in such a test may be free in solution, affixed to a solid support, borne on a cell surface, or located intracellularly. The assay of the present invention is a cell based assay.

The assay of the present invention may be a screen, whereby a number of agents are tested. In one aspect, the assay method of the present invention is a high through put screen.

Techniques for drug screening may be based on the method described in Geysen, European Patent No. 0138855, published on Sep. 13, 1984. In summary, large numbers of different small peptide candidate modulators are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators.

This invention also contemplates the use of competitive drug screening assays in which neutralising antibodies capable of binding a target specifically compete with a test compound for binding to a target.

It is expected that the assay methods of the present invention will be suitable for both small and large-scale screening of test compounds as well as in quantitative assays.

Various nucleic acid assays are also known. Any conventional technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

Target gene presence, amplification and/or expression may be measured in a sample directly, for example, by conventional Southern blotting, Northern blotting to quantitate the transcription of target mRNA, dot blotting (DNA or RNA analysis), or in situ hybridisation, using an appropriately labelled probe. Those skilled in the art will readily envisage how these methods may be modified, if desired.

Generation of nucleic acids for analysis from samples generally requires nucleic acid amplification. Many amplification methods rely on an enzymatic chain reaction (such as a polymerase chain reaction, a ligase chain reaction, or a self-sustained sequence replication) or from the replication of all or part of the vector into which it has been cloned. Preferably, the amplification according to the invention is an exponential amplification, as exhibited by for example the polymerase chain reaction.

Many target and signal amplification methods have been described in the literature, for example, general reviews of these methods in Landegren, U., et al., Science 242:229-237 (1988) and Lewis, R., Genetic Engineering News 10:1, 54-55 (1990). These amplification methods may be used in the methods of our invention, and include polymerase chain reaction (PCR), PCR in situ, ligase amplification reaction (LAR), ligase hybridisation, Qbeta bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

PCR is a nucleic acid amplification method described inter alia in U.S. Pat. Nos. 4,683,195 and 4,683,202. PCR consists of repeated cycles of DNA polymerase generated primer extension reactions. PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached. PCR can be used to amplify any known nucleic acid in a diagnostic context (Mok et al., (1994), Gynaecologic Oncology, 52: 247-252).

Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ have been achieved in one hour at 42° C.

Ligation amplification reaction or ligation amplification system uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions.

In the presence of two suitably designed primers, a geometric amplification occurs via DNA strand displacement and hyperbranching to generate 10¹² or more copies of each circle in 1 hour.

If a single primer is used, RCAT generates in a few minutes a linear chain of thousands of tandemly linked DNA copies of a target covalently linked to that target.

A further technique, strand displacement amplification (SDA; Walker et al., (1992) PNAS (USA) 80:392) begins with a specifically defined sequence unique to a specific target. But unlike other techniques which rely on thermal cycling, SDA is an isothermal process that utilises a series of primers, DNA polymerase and a restriction enzyme to exponentially amplify the unique nucleic acid sequence.

SDA comprises both a target generation phase and an exponential amplification phase.

In target generation, double-stranded DNA is heat denatured creating two single-stranded copies. A series of specially manufactured primers combine with DNA polymerase (amplification primers for copying the base sequence and bumper primers for displacing the newly created strands) to form altered targets capable of exponential amplification.

The exponential amplification process begins with altered targets (single-stranded partial DNA strands with restricted enzyme recognition sites) from the target generation phase.

An amplification primer is bound to each strand at its complementary DNA sequence. DNA polymerase then uses the primer to identify a location to extend the primer from its 3′ end, using the altered target as a template for adding individual nucleotides. The extended primer thus forms a double-stranded DNA segment containing a complete restriction enzyme recognition site at each end.

A restriction enzyme is then bound to the double stranded DNA segment at its recognition site. The restriction enzyme dissociates from the recognition site after having cleaved only one strand of the double-sided segment, forming a nick. DNA polymerase recognises the nick and extends the strand from the site, displacing the previously created strand. The recognition site is thus repeatedly nicked and restored by the restriction enzyme and DNA polymerase with continuous displacement of DNA strands containing the target segment.

Each displaced strand is then available to anneal with amplification primers as above. The process continues with repeated nicking, extension and displacement of new DNA strands, resulting in exponential amplification of the original DNA target.

In an alternative embodiment, the present invention provides for the detection of gene expression at the RNA level. Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

PCR technology as described e.g. in section 14 of Sambrook et al., 1989, requires the use of oligonucleotide probes that will hybridise to target nucleic acid sequences. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating ³²P DATP with the Klenow fragment of DNA polymerase in a random priming reaction, as is well known in the art. Oligonucleotides are usually end-labelled with ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68° C. High stringency conditions can be provided, for example, by hybridisation in an aqueous solution containing 6×SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefor easily identifiable. Thus, cell-based screening assays can be designed by constructing cell lines in which the expression of a reporter protein, i.e. an easily assayable protein, such as β-galactosidase, chloramphenicol acetyltransferase (CAT) or luciferase, is dependent on the activation of a Notch. For example, a reporter gene encoding one of the above polypeptides may be placed under the control of an response element which is specifically activated by Notch signalling. Alternative assay formats include assays which directly assess responses in a biological system. If a cell-based assay system is employed, the test compound(s) indentified may then be subjected to in vivo testing to determine their effect on Notch signalling pathway.

In general, reporter constructs useful for detecting Notch signalling by expression of a reporter gene may be constructed according to the general teaching of Sambrook et al (1989). Typically, constructs according to the invention comprise a promoter of the gene of interest (i.e. of an endogenous target gene), and a coding sequence encoding the desired reporter constructs, for example of GFP or luciferase. Vectors encoding GFP and luciferase are known in the art and available commercially.

Sorting of cells, based upon detection of expression of target genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure target gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefor generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefor assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a target mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In Vivo Expression Technology (IVET) (Camilli). IVET identifies target genes up-regulated during say treatment or disease when compared to laboratory culture.

The present invention also provides a method of detection of polypeptides. The advantage of using a protein assay is that Notch activation can be directly measured. Assay techniques that can be used to determine levels of a polypeptide are well known to those skilled in the art. Such assay methods include radioimmunoassays, competitive-binding assays, protein gel assay, Western Blot analysis, antibody sandwich assays, antibody detection, FACS and ELISA assays. For example, polypeptides can be detected by differential mobility on protein gels, or by other size analysis techniques, such as mass spectrometry. The detection means may be sequence-specific. For example, polypeptide or RNA molecules can be developed which specifically recognise polypeptides in vivo or in vitro.

For example, RNA aptamers can be produced by SELEX. SELEX is a method for the in vitro evolution of nucleic acid molecules with highly specific binding to target molecules. It is described, for example, in U.S. Pat. Nos. 5,654,151, 5,503,978, 5,567,588 and 5,270,163, as well as PCT publication WO 96/38579

The invention, in certain embodiments, includes antibodies specifically recognising and binding to polypeptides.

Antibodies may be recovered from the serum of immunised animals. Monoclonal antibodies may be prepared from cells from immunised animals in the conventional manner.

The antibodies of the invention are useful for identifying cells expressing the genes being monitored.

Antibodies according to the invention may be whole antibodies of natural classes, such as IgE and IgM antibodies, but are preferably IgG antibodies. Moreover, the invention includes antibody fragments, such as Fab, F(ab′)2, Fv and ScFv. Small fragments, such Fv and ScFv, possess advantageous properties for diagnostic and therapeutic applications on account of their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualisable within tissues. Moreover, they may be fluorescent labels or other labels which are visualisable in tissues and which may be used for cell sorting.

In more detail, antibodies as used herein can be altered antibodies comprising an effector protein such as a label. Especially preferred are labels which allow the imaging of the distribution of the antibody in vivo. Such labels can be radioactive labels or radioopaque labels, such as metal particles, which are readily visualisable within the body of a patient. Moreover, they can be fluorescent labels or other labels which are visualisable on tissue

Antibodies as described herein can be produced in cell culture. Recombinant DNA technology can be used to produce the antibodies according to established procedure, in bacterial or preferably mammalian cell culture. The selected cell culture system optionally secretes the antibody product, although antibody products can be isolated from non-secreting cells.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by an enzyme immunoassay, e.g. a sandwich assay or a dot-assay, or a radioimmunoassay.

For isolation of the antibodies, the immunoglobulins in the culture supernatants or in the ascitic fluid can be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target antigen, or with Protein-A.

The antibody is preferably provided together with means for detecting the antibody, which can be enzymatic, fluorescent, radioisotopic or other means. The antibody and the detection means can be provided for simultaneous, simultaneous separate or sequential use, in a kit.

The antibodies of the invention are assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al, eds, 1994, Current Protocols in Molecular Biology, Vol. 1, John Wiley & Sons, Inc., New York, which is incorporated by reference herein in its entirety). Exemplary immunoassays are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2,1% Trasylol) supplemented with protein phosphatase and/or protease inhibitors (e. g., EDTA, PMSF, aprotinin, sodium vanadate), adding the antibody of interest to the cell lysate, incubating for a period of time (e. g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e. g., western blot analysis.

Western blot analysis generally comprises preparing protein samples, electrophoresis of the protein samples in a polyacrylamide gel (e. g., 8%-20% SDS-PAGE depending on the molecular weight of the antigen), transferring the protein sample from the polyacrylamide gel to a membrane such as nitrocellulose, PVDF or nylon, blocking the membrane in blocking solution (e. g., PBS with 3% BSA or non-fat milk), washing the membrane in washing buffer (e. g., PBS-Tween 20), exposing the membrane to a primary antibody (the antibody of interest) diluted in blocking buffer, washing the membrane in washing buffer, exposing the membrane to a secondary antibody (which recognises the primary antibody, e. g., an antihuman antibody) conjugated to an enzymatic substrate (e. g., horseradish peroxidase or alkaline phosphatase) or radioactive molecule (e. g., ³²P or 125I) diluted in blocking buffer, washing the membrane in wash buffer, and detecting the presence of the antigen.

ELISAs generally comprise preparing antigen, coating the well of a 96 well microtitre plate with the antigen, adding the antibody of interest conjugated to a detectable compound such as an enzymatic substrate (e. g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognises the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well.

It is convenient when running assays to immobilise one of more of the reactants, particularly when the reactant is soluble. In the present case it may be convenient to immobilse any one of more of the candidate modulator, Notch ligand, immune cell activator or immune cell costimulus. Immobilisation approaches include covalent immobilsation, such as using amine coupling, surface thiol coupling, ligand thiol coupling and aldehyde coupling, and high affinity capture which relies on high affinity binding of a ligand to an immobilsed capturing molecule. Example of capturing molecules include: streptavidin, anti-mouse Ig antibodies, ligand-specific antibodies, protian A, protein G and Tag-specific capture. In one embodiment, immobilisation is achieved through binding to a support, particularly a particulate support which is preferably in the form of a bead.

For assays involving monitoring or detection of tolerised T-cells for use in clinical applications, the assay will generally involve removal of a sample from a patient prior to the step of detecting a signal resulting from cleavage of the intracellular domain.

The invention additionally provides a method of screening for a candidate modulator of Notch signalling, the method comprising mixing in a buffer an appropriate amount of Notch, wherein Notch is suitably labelled with detection means for monitoring cleavage of Notch; and a sample of a candidate ligand; and monitoring any cleavage of Notch.

As used herein, the term “sample” refers to a collection of inorganic, organic or biochemical molecules which is either found in nature (e.g., in a biological- or other specimen) or in an artificially-constructed grouping, such as agents which may be found and/or mixed in a laboratory. The biological sample may refer to a whole organism, but more usually to a subset of its tissues, cells or component parts (e.g. body fluids, including but not limited to blood, mucus, saliva and urine).

The present invention provides a method of detecting novel modulators of Notch signalling. The modulators identified may be used as therapeutic agents—i.e. in therapy applications.

TH2 Modulation

The humoral/TH2 branch of the immune system is generally directed at protecting against extracellular immunogens such as bacteria and parasites through the production of antibodies by B cells; whereas the cellular/TH1 branch is generally directed at intracellular immunogens such as viruses and cancers through the activity of natural killer cells, cytotoxic T lymphocytes and activated macrophages (U.S. Pat. No. 6,039,969). TH2 cells are believed to produce cytokines which stimulate production of IgE antibodies, as well as to be involved with recruitment, proliferation, differentiation, maintenance and survival of eosinophils, which can result in eosinophilia. Eosinophilia is a hallmark of many TH2 mediated diseases, such as asthma, allergy, and atopic dermatitis.

Some diseases that are thought to be caused/mediated in substantial part by TH2 immune response, IL-4/IL-5 cytokine induction, and/or eosinophilia include asthma, allergic rhinitis, systemic lupus erythematosis, Ommen's syndrome (hypereosinophilia syndrome), certain parasitic infections, for example, cutaneous and systemic leishmaniasis, toxoplasma infection and trypanosome infection, and certain fungal infections, for example candidiasis and histoplasmosis, and certain intracellular bacterial infections, such as leprosy and tuberculosis. Additionally, it should also be noted that diseases having a viral or cancer related basis, but with a significant TH2 mediated pathology can also be beneficially treated according to the present invention.

Recent evidence indicates that the immune system can be broken down into two major arms, the humoral and cellular arms. The humoral arm is important in eliminating extracellular pathogens such as bacteria and parasites through production of antibodies by B cells. On the other hand, the cellular arm is important in the elimination of intracellular pathogens such as viruses through the activity of natural killer cells, cytotoxic T lymphocytes and activated macrophages. In recent years it has become apparent that these two arms are activated through distinct T helper cell (TH) populations and their distinct cytokine production profiles. T helper type 1 (TH1) cells are believed to enhance the cellular arm of the immune response and produce predominately the cytokines IL-2 and IFN-.gamma.; whereas, T helper 2 (TH2) cells are believed to enhance the humoral arm of the immune response and produce cytokines, such as interleukin-3 (IL-3), interleukin-4 (IL-4), interleukin-5 (IL-5) and granulocyte-macrophage colony-stimulating factor (GM-CSF). In the TH2 case, IL-3, IL-5 and GM-CSF are thought to stimulate eosinophilopoiesis. In addition, IL-5 facilitates terminal differentiation and cell proliferation of eosinophils and promotes survival, viability and migration of eosinophils, while IL-4 stimulates production of antibodies of the IgE class. IgE is an important component in allergies and asthma. IL-5 may also prime eosinophils for the subsequent actions of other mediators.

In contrast, the TH1 cytokines, IL-2 and IFN gamma., are important in activating macrophages, NK cells and CTL (cytotoxic T lymphocytes). IFN gamma also stimulates B cells to secrete specifically cytophilic antibody for the elimination of virally-infected cells. Interestingly, IFN alpha a macrophage-derived cytokine has been shown to antagonize TH2-type responses. IFN alpha also appears to inhibit the proliferation and cytokine production of TH2 cells and enhances IFN gamma production by TH1 cells. In addition, IFN alpha also appears to inhibit IgE production and antigen-induced increases in IL4 mRNA levels.

One common feature of many TH2 mediated diseases is an accumulation of eosinophils, referred to as eosinophilia. For example, chronic pulmonary inflammation involving eosinophil infiltration is a characteristic hallmark feature of bronchial asthma. Increased numbers of eosinophils have been observed in blood, bronchoalveolar lavage fluid and pulmonary tissue in patients with asthma, but the mechanism(s) responsible for their recruitment into and regulation within pulmonary tissues undergoing allergic or pro-inflammatory reactions has not been fully understood. Mediators and cytokines from T-lymphocytes and effector cells such as basophils, mast cells, macrophages and eosinophils have been implicated in enhancing cell maturation, chemotaxis and activation of eosinophils. Evidence suggests that an association exists between the immune system, especially CD4+ T cells, and eosinophils and eosinophil recruitment. Studies in asthmatics and in animal models of allergic pulmonary responses support this notion with the evidence of close correlations between the relative numbers of T cells and activated eosinophils in the airways.

Examples of diseases which may be treated by reducing a TH2 response according to the present invention include include asthma, allergy, atopic dermatitis, early HIV disease, infectious mononucleosis, systemic lupus erythematosis, parasitic infections, for example, cutaneous and systemic leishmaniasis, Toxoplasma infection and Trypanosome infection, certain fungal infections, for example Candidiasis and Histoplasmosis, and intracellular bacterial infections, such as leprosy and tuberculosis.

TNF Modulation

At least two TNFs have been previously described, specifically TNF alpha (TNF alpha) and TNF beta (TNF beta or lymphotoxin), and each is active as a trimeric molecule and is believed to initiate cellular signaling by crosslinking receptors (Engelmann et al. (1990), J. Biol. Chem., 265:14497-14504).

Several lines of evidence implicate TNF alpha and TNF beta as major inflammatory cytokines. These known TNFs have important physiological effects on a number of different target cells which are involved in inflammatory responses to a variety of stimuli such as infection and injury. The proteins cause both fibroblasts and synovial cells to secrete latent collagenase and prostaglandin E2 and cause osteocyte cells to stimulate bone resorption. These proteins increase the surface adhesive properties of endothelial cells for neutrophils. They also cause endothelial cells to secrete coagulant activity and reduce their ability to lyse clots. In addition they redirect the activity of adipocytes away from the storage of lipids by inhibiting expression of the enzyme lipoprotein lipase. TNFs also cause hepatocytes to synthesize a class of proteins known as “acute phase reactants,” which act on the hypothalamus as pyrogens (Selby et al. (1988), Lancet, 1 (8583):483; Starnes, Jr. et al. (1988), J. Clin. Invest., 82:1321; Oliff et al. (1987), Cell, 50:555; and Waage et al. (1987), Lancet, 1 (8529):355). Particular examples of diseases which may be treated according to the present invention include, for example:
(A) acute and chronic immune and autoimmune pathologies, such as systemic lupus erythematosus (SLE) rheumatoid arthritis, rheumatoid spondylitis, osteoarthritis, gouty arthritis and other arthritic conditions, thyroidosis, graft versus host disease, scleroderma, diabetes mellitus, Graves' disease, Beschet's disease, and the like;
(B) infections, including, but not limited to, sepsis syndrome, general sepsis, gram-negative sepsis, septic shock, endotoxic shock, toxic shock syndrome, cachexia, circulatory collapse and shock resulting from acute or chronic bacterial infection, acute and chronic parasitic and/or infectious diseases, bacterial, viral or fungal, such as a HIV, AIDS (including symptoms of cachexia, autoimmune disorders, AIDS dementia complex and infections), fever and myalgias due to bacterial or viral infections;
(C) inflammatory diseases, such as chronic inflammatory pathologies and vascular inflammatory pathologies, including chronic inflammatory pathologies such as sarcoidosis, chronic inflammatory bowel disease, ulcerative colitis, and Crohn's pathology and vascular inflammatory pathologies, such as, but not limited to, disseminated intravascular coagulation, atherosclerosis, and Kawasaki's pathology;
(D) neurodegenerative diseases, including, but are not limited to, demyelinating diseases, such as multiple sclerosis and acute transverse myelitis; extrapyrarnidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo palsy; Cerebellar and Spinocerebellar Disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and Machadojoseph)); and systemic disorders (Refsum's disease, abetalipoprotemia, ataxia, telangiectasia, and mitochondrial multi system disorder); demyelinating core disorders, such as multiple sclerosis, acute transverse myelitis; disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wemicke-Korsakoff syndrome; chronic alcoholism; Creutzfeldt-Jakob disease; Subacute sclerosing panencephalitis, Hallerrorden-Spatz disease; and Dementia pugilistica, or any subset thereof;
(E) malignant pathologies involving TNF-secreting tumors or other malignancies involving TNF, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or Mycosis fungoides)); carcinomas (such as colon carcinoma) and metastases thereof; cancer-related angiogenesis; infantile haemangiomas;
(F) alcohol-induced hepatitis; and
(G) other diseases related to angiogenesis or VEGF/VPF, such as ocular neovascularization, psoriasis, duodenal ulcers, angiogenesis of the female reproductive tract;
(H) cardiovascular conditions such as atherosclerosis, congestive heart failure, stroke and vasculitis;
(I) pulmonary diseases such as adult respiratory distress syndrome (ARDS), chronic pulmonary inflammatory disease, silicosis, asbestosis and pulmonary sarcoidosis.

In one embodiment the present invention may be used to treat a “TNF-mediated disease” A disease or medical condition may be considered to be a “TNF-mediated disease” if the spontaneous or experimental disease is associated with elevated levels of TNF in bodily fluids or in tissues adjacent to the focus of the disease or indication within the body.

Diseases such as rheumatoid arthritis and psoriatic arthritis are chronic joint diseases that afflict and disable, to varying degrees, millions of people worldwide. Rheumatoid arthritis is a disease of articular joints in which the cartilage and bone are slowly eroded away by a proliferative, invasive connective tissue called pannus, which is derived from the synovial membrane. The disease may involve peri-articular structures such as bursae, tendon sheaths and tendons as well as extra-articular tissues such as the subcutis, cardiovascular system, lungs, spleen, lymph nodes, skeletal muscles, nervous system (central and peripheral) and eyes (Silberberg (1985), Anderson's Pathology, Kissane (ed.), II:1828).

There is a wide spectrum of disease severity, but many patients run a course of intermittent relapses and remissions with an overall pattern of slowly progressive joint destruction and deformity. The clinical manifestations may include symmetrical polyarthritis of peripheral joints with-pain, tenderness, swelling and loss of function of affected joints; morning stiffness; and loss of cartilage, erosion of bone matter and subluxation of joints after persistent inflammation. Extra-articular manifestations include rheumatoid nodules, rheumatoid vasculitis, pleuropulmonary inflammations, scleritis, sicca syndrome, Felty's syndrome (splenomegaly and neutropenia), osteoporosis and weight loss (Katz (1985), Am. J. Med., 79:24 and Krane and Simon (1986), Advances in Rheumatology, Synderman (ed.), 70(2):263-284). The clinical manifestations result in a high degree of morbidity resulting in disturbed daily life of the patient.

Therapy

The term “therapy” includes curative effects, alleviation effects, and prophylactic effects. The therapy may be on humans or animals.

Modulators identified by the assay method of the present invention may be used to treat disorders and/or conditions of the immune system. In particular, the compounds can be used in the treatment of T cell mediated diseases or disorders. A detailed description of the conditions affected by the Notch signalling pathway may be found in our WO98/20142, WO00/36089 and WO/00135990.

Diseased or infectious states that may be described as being mediated by T cells include, but are not limited to, any one or more of asthma, allergy, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple sclerosis, rheumatoid arthritis and diabetes. The present invention may also be used in organ transplantation or bone marrow transplantation. The present invention is also useful in treating immune disorders such as autoimmune disorders or graft rejection such as allograft rejection.

Examples of autoimmune disorders range from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail, organ-specific autoimmune diseases include multiple sclerosis, insulin dependent diabetes mellitus, several forms of anemia (aplastic, hemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in cancer therapy, particularly in diseases involving the conversion of epithelial cells to cancer. In particular, the invention may be useful in increasing immune response to cancer by modulating production of key cytokines, for example by use of an inhibitor of Notch signalling. The present invention is especially useful in relation to adenocarcinomas such as: small cell lung cancer, and cancer of the kidney, uterus, prostrate, bladder, ovary, colon and breast. Thus, the present application has application in the treatment of malignant and pre-neoplastic disorders. The present invention is especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breasy cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

Pharmaceutical Compositions

The present invention provides a pharmaceutical composition comprising administering a therapeutically effective amount of at least one compound identified by the method of the present invention and a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the compound is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration.

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular, intradermal, intra-articular, intrathecal, intra-peritoneal or subcutaneous route, or via the alimentary tract (for example, via the Peyers patches).

The routes of administration and dosages described are intended only as a guide since a skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more substances capable of up-regulating or down-regulating the Notch signalling pathway are then typically added to the culture medium together with the antigen of interest. The antigen may be added before, after or at substantially the same time as the substance(s). Cells are typically incubated with the substance(s) and antigen for at least one hour, preferably at least 3 hours, if necessary for at least 12 hours or more at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of a Notch signalling are now ready for use.

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s) capable of modulating Notch to that used with the APC, then the T cell will not be brought into contact with the different substance(s) used in the APC. Alternatively, the T cell may be incubated with a first substance (or set of substances) to modulate Notch signalling, washed, resuspended and then incubated with the primed APC in the absence of both the substance(s) used to modulate the APC and the substance(s) used to modulate the T cell. Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Incubations will typically be for at least 1 hour, preferably at least 3 or 6 hours, in suitable culture medium at 37° C. Induction of immunotolerance may be determined by subsequently challenging T cells with antigen and measuring IL-2 production compared with control cells not exposed to APCs.

T cells or B cells which have been primed in this way may be used according to the invention to induce immunotolerance in other T cells or B cells.

The present invention is additionally described by way of the following illustrative, non-limiting Examples, which provide a better understanding of the present invention and of its many advantages.

### EXAMPLES

### Example 1

### CD4+ Cell Purification

Spleens were removed from mice (variously Balb/c females, 8-10 weeks, C57B/6 females, 8-10 weeks, CARD1 females, 8-10 weeks (D011.10 transgenic, CAR transgenic)) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHC0₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

### Example 2

### Antibody Coating

The following protocols were used for coating 96 well flat-bottomed plates with antibodies.

A) The plates were coated with Dulbecco's Phosphate Buffered Saline (DPBS) plus 1 μg/ml anti-CD3 antibody (Pharmingen, San Diego, US: Cat No 553058, Clone No 145-2C11) plus 1 μg/ml anti-IgG4 antibody (Pharmingen Cat No 555878). 100 μl of coating mixture was used per well. Plates were incubated overnight at 4° C. then washed with DPBS. Each well then received either 100 μl DPBS or 100 μl DPBS plus 10 μg/ml Notch ligand (mouse Delta 1 extracellular domain/Ig4Fc fusion protein; Fc-delta).

The plates were incubated for 2-3 hours at 37° C. then washed again with DPBS before cells (prepared as in Example 1) were added.

B) Alternatively, the plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus Fc-delta (10 μg/ml). The plates were incubated for 2-3 hours at 37° C. then washed again with DPBS before cells (prepared as in Example 1) were added.

### Example 3

### Primary Polyclonal Stimulation

CD4+ cells were cultured in 96 well, flat-bottomed plates pre-coated according to Example 2(A) or 2(B). Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of R10F medium was then added to each well to give a final volume of 200 μl (2×10⁵ cells/well, anti-CD28 final concentration 2 μg/ml) The plates were then incubated at 37° C. for 72 hours.

125 μl supernatant was then removed from each well and stored at −20° C. until tested by ELISA for IL-10, IFNg and IL-13using antibody pairs from R & D Systems (Abingdon, UK). The cells were then split 1 in 3 into new wells (not coated) and fed with R10F medium plus recombinant human IL-2 (2.5 ng/ml, PeproTech Inc, London, UK: Cat No 200-02).

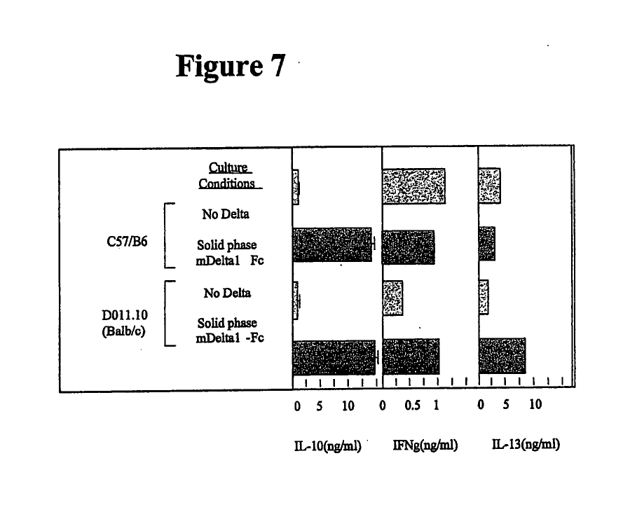

Results are shown in FIG. 7.

### Example 4

### Real Time PCR Analysis of Primary Stimulated CD4+ Cells

Murine (Balb/c) stimulated CD4⁺ T-cells from Example 3 were harvested at 4, 16 and 24 hours. Total cellular RNA was isolated using the RNeasy™ RNA isolation kit (Qiagen, Crawley, UK) according to the manufacturer's guidelines.

In each case 1 μg of total RNA was reverse transcribed using SuperScript™ II Reverse Transcriptase (Invitrogen, Paisley, UK) using Oligo dT₍₁₂₋₁₈₎ or a random decamer mix according to the manufacturer's guidelines. After synthesis, Oligo dT₍₁₂₋₁₈₎₋ and random decamer-primed cDNAs were mixed in equal proportions to provide the working cDNA sample for real-time quantitative PCR analysis.

Real-time quantitative PCR was performed using the Roche Lightcycler™ system (Roche, UK) and SYBR green detection chemistry according to the manufacturer's guidelines. The following HPLC-purified primer pairs were used for cDNA-specific amplification (5′ to 3′):

mouse 18s rRNA:

| | |
|---|---|
| Forward | GTAACCCGTTGAACCCCATT |
| Reverse | CCATCCAATCGGTAGTAGCG |

mouse Hes-1:

| | |
|---|---|
| Forward | GGTGCTGATAACAGCGGAAT |
| Reverse | ATTTTTGGAATCCTTCACGC |

The endpoint used in real-time PCR quantification, the Crossing Point (Cₚ), is defined as the PCR cycle number that crosses an algorithm-defined signal threshold. Quantitative analysis of gene-specific cDNA was achieved firstly by generating a set of standards using the Cₚs from a set of serially-diluted gene-specific amplicons which had been previously cloned into a plasmid vector (pCR2.1, Invitrogen). These serial dilutions fall into a standard curve against which the Cₚs from the cDNA samples were compared. Using this system, expression levels of the 18S rRNA house-keeping gene were generated for each cDNA sample. Hes-1 was then analysed by the same method using serially-diluted Hes-1-specific standards, and the Hes-1 value divided by the 18S rRNA value to generate a value, which represents the relative expression of Hes-1 in each cDNA sample. All Cp analysis was performed using the Second Derivative Maximum algorithm within the Lightcycler system software.

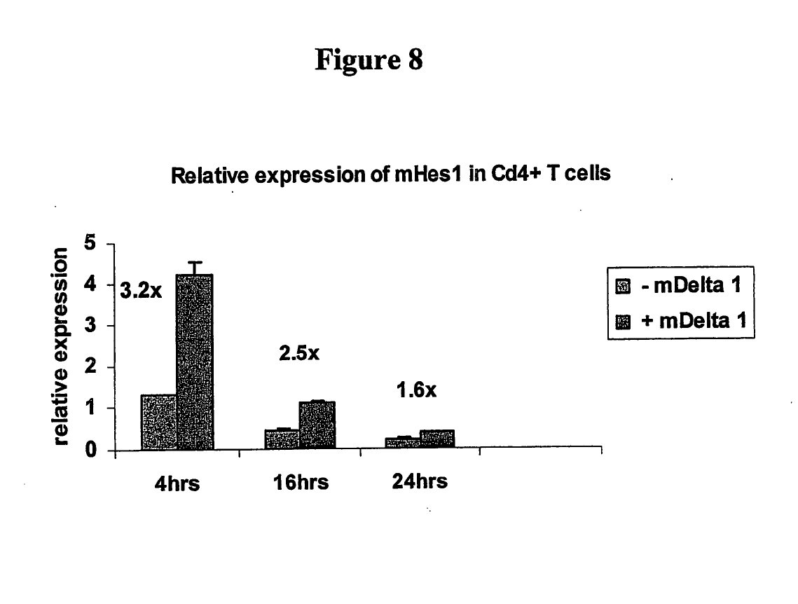

Results (HES-1 expression relative to 18S rRNA expression with and without Fc-delta) are shown in FIG. 8.

### Example 5

### Screening Under Polarising Conditions

Plates were coated and CD4+ cells added as in Example 2(A).

The procedure of Example 3 was then followed, except that instead of adding 100 μl R10F medium per well as in Example 3, 100 μl of polarising cocktail was added per well as follows:

Un-polarised cells: R10F medium.

Th1 polarised cells: R10F medium plus anti-IL-4 antibody (10 μg/ml, Pharmingen Cat No 554432) plus IL-12 (10 ng/ml, Peprotech 210-12).

Th2 polarised cells: R10F medium plus anti-IL-12 antibody (10 μg/ml, Pharmingen Cat No 554475) plus anti-IFNg antibody (1 μg/ml, Pharmingen Cat No 554408) plus IL-4 (10 ng/ml, Peprotech Cat No 214-14).

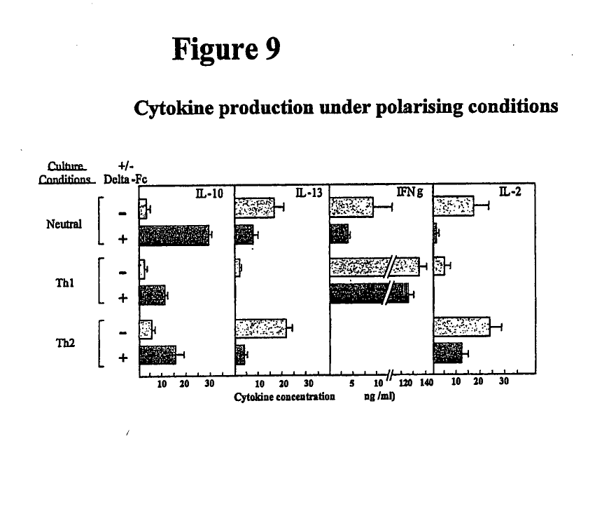

Cells were then stimulated and cytokines (IL-10, IFNγ and IL-13) measured by ELISA as described in Example 3. Results are shown in FIG. 9.

### Example 6

### Soluble Ligand

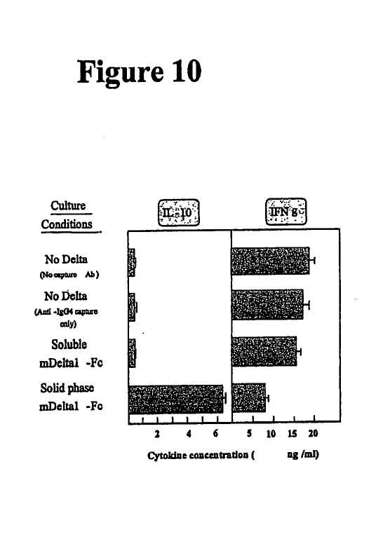

The procedure of Example 2(A) (with the modification that ligand was not added to the plate) and Example 3 (with the modification that soluble Fc-delta was added with the R10F medium) was used to compare soluble Fc-delta with plate-bound Fc-delta against controls. Results are shown in FIG. 10.

### Example 7

Secondary Stimulation

7 days after primary stimulation all cells were harvested and counted then stimulated in one of three ways as follows:

Re-Stimulation

Cells were re-stimulated exactly as for primary stimulation (Example 3).

Re-challenge on Anti-CD3/CD28

96-well flat-bottomed plates were coated with PBS plus 1 μg/ml anti-CD3 antibody. The plates were incubated overnight at 4° C. then washed with DPBS.

The cells were re-suspended at 2×10⁶/ml in R10F medium plus anti-CD28 antibody (4 μg/ml). 100 μl cell suspension was added per well. 100 μl of R10F medium was then added per well to give a final volume of 200 μl. (2×10⁵ cells/well, anti-CD28 final concentration 2 μg/ml). The plates were then incubated at 37° C. for 72 hours. After 72 hours supernatants were removed for ELISA as described in Example 3 (primary stimulation).

Re-Stimulation With APC Plus Anti-CD3

Primary stimulated cells from Example 3 were harvested after 7 days and restimulated with APCs of the same strain (2×10⁴ per well) plus anti-CD3 antibody.

Mouse spleen cells were isolated as described in Example 1 up to the counting step. Thy-1.2 antibody-binding cells were then removed on a MACS column and the flowthrough was recovered and treated with mitomycin-C for 45 minutes then added to a 96 well plate in 100 μl R10F medium with equal numbers of cells from Example 3 and 0.5 μg/ml anti-CD3 antibody.

Cell proliferation was measured using a kit from Roche Molecular Biochemicals, Cell Proliferation ELISA, BrdU (chemiluminescent) 1 669 915, according to the manufacturer's instructions. Plates were pulsed at 72 hours and read on a luminometer.

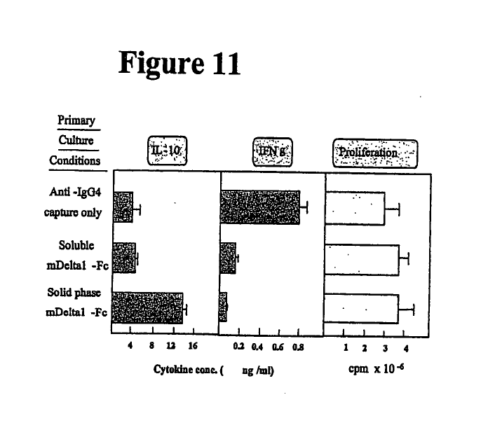

Cytokines (IL-10 and IFN-γ) were measured as described in Example 3. Results are shown in FIG. 11.

### Example 8

### CHO-N2 (N27) Luciferase Reporter Assay

A) Construction of Luciferase Reporter Plasmid 10×CBF1-Luc (pLOR91)

An adenovirus major late promoter TATA-box motif with BgiII and HindIII cohesive ends was generated as follows:

BglII HindIII
GATCTGGGGGGCTATAAAAGGGGGTA
ACCCCCCGATATTTTCCCCCATTCGA

This was cloned into plasmid pGL3-Basic (Promega) between the BgiII and HindIII sites to generate plasmid pGL3-AdTATA.

A TP1 promoter sequence (TP1; equivalent to 2 CBF1 repeats) with BamH1 and BglII cohesive ends was generated as follows:

BamH1 BglII
5′ GATCCCGACTCGTGGGAAAATGGGCGGAAGGGCACCGTGGGAAAATAGTA 3′
3′ GGCTGAGCACCCTTTTACCCGCCTTCCCGTGGCACCCTTTTATCATCTAG 5′

This sequence was pentamerised by repeated insertion into a BglII site and the resulting TP1 pentamer (equivalent to 10 CBF1 repeats) was inserted into pGL3-AdTATA at the BglII site to generate plasmid pLOR91.

B) Generation of a Stable CHO Cell Reporter Cell Line Expressing Full Length Notch2 and the 10×CBF1-Luc Reporter Cassette

A cDNA clone spanning the complete coding sequence of the human Notch2 gene (see, eg GenBank Accession No AF315356) was constructed as follows. A 3′ cDNA fragment encoding the entire intracellular domain and a portion of the extracellular domain was isolated from a human placental cDNA library (OriGene Technologies Ltd., USA) using a PCR-based screening strategy. The remaining 5′ coding sequence was isolated using a RACE (Rapid Amplification of cDNA Ends) strategy and ligated onto the existing 3′ fragment using a unique restriction site common to both fragments (Cla I). The resulting full-length cDNA was then cloned into the mammalian expression vector pcDNA3.1-V5-HisA (Invitrogen) without a stop codon to generate plasmid pLOR92. When expressed in mammalian cells, pLOR92 thus expresses the full-length human Notch2 protein with V5 and His tags at the 3′ end of the intracellular domain.

Wild-type CHO-K1 cells (eg see ATCC No CCL 61) were transfected with pLOR92 (pcDNA3.1-FLNotch2-V5-His) using Lipfectamine 2000™ (Invitrogen) to generate a stable CHO cell clone expressing full length human Notch2 (N2). Transfectant clones were selected in Dulbecco's Modified Eagle Medium (DMEM) plus 10% heat inactivated fetal calf serum ((HI)FCS) plus glutamine plus Penicillin-Streptomycin (P/S) plus 1 mg/ml G418 (Geneticin™—Invitrogen) in 96-well plates using limiting dilution. Individual colonies were expanded in DMEM plus 10% (HI)FCS plus glutamine plus P/S plus 0.5 mg/ml G418. Clones were tested for expression of N2 by Western blots of cell lysates using an anti-V5 monoclonal antibody (Invitrogen). Positive clones were then tested by transient transfection with the reporter vector pLOR91 (10×CBF1-Luc) and co-culture with a stable CHO cell clone (CHO-Delta) expressing full length human delta-like ligand 1 (DLL1; eg see GenBank Accession No AF196571). (CHO-Delta was prepared in the same way as the CHO Notch 2 clone, but with human DLL1 used in place of Notch 2. A strongly positive clone was selected by Western blots of cell lysates with anti-V5 mAb.)

One CHO-N2 stable clone, N27, was found to give high levels of induction when transiently transfected with pLOR91 (10×CBF1-Luc) and co-cultured with the stable CHO cell clone expressing full length human DLL1 (CHO-Delta1). A hygromycin gene cassette (obtainable from pcDNA3.1/hygro, Invitrogen) was inserted into pLOR91 (10×CBF1-Luc) using BamH1 and Sal1 and this vector (10×CBF1-Luc-hygro) was transfected into the CHO-N2 stable clone (N27) using Lipfectamine 2000 (Invitrogen). Transfectant clones were selected in DMEM plus 10% (HI)FCS plus glutamine plus P/S plus 0.4 mg/ml hygromycin B (Invitrogen) plus 0.5 mg/ml G418 (Invitrogen) in 96-well plates using limiting dilution. Individual colonies were expanded in DMEM plus 10% (HI)FCS plus glutamine plus P/S+0.2 mg/ml hygromycin B plus 0.5 mg/ml G418 (Invitrogen).

Clones were tested by co-culture with a stable CHO cell clone expressing FL human DLL1. Three stable reporter cell lines were produced N27#11, N27#17 and N27#36. N27#11 was selected for further use because of its low background signal in the absence of Notch signalling, and hence high fold induction when signalling is initiated. Assays were set up in 96-well plates with 2×10⁴ N27#11 cells per well in 100 μl per well of DMEM plus 10% (HI)FCS plus glutamine plus P/S.

C) Transient Transfection of CHO-N2 Cells With 10×CBF1-Luc

Alternatively, for transient transfection, CHO-N2 (Clone N27) cells were maintained in DMEM plus 10% (HI)FCS plus glutamine plus P/S plus 0.5 mg/ml G418 and a T₈₀ flask of the CHO-N2 cells was transfected as follows. The medium on the cells was replaced with 8 ml of fresh in DMEM plus 10% (HI)FCS plus glutamine plus P/S. In a sterile bijou 10 μg of pLOR91 (10×CBF1-Luc) was added to OptiMem (Invitrogen) to give a final volume of 1 ml and mixed. In a second sterile bijou 20 μl of Lipofectamine 2000 reagent was added to 980 μl of OptiMem and mixed.

The contents of each bijou were mixed and left at room temperature for 20 minutes. The 2 ml of transfection mixture was added to the flask of cells containing 8 ml of medium and the resulting mixture was left in a CO₂ incubator overnight before removing the transfected cells and adding to the 96-well plate containing the immobilised Notch ligand protein.

The following day the transfected CHO-N2 cells were removed using 0.02% EDTA solution (Sigma), spun down and resuspended in 10 ml DMEM plus 10% (HI)FCS plus glutamine plus P/S. 10 μl of cells were counted and the cell density was adjusted to 2.0×10⁵ cells/ml with fresh DMEM plus 10% (HI)FCS plus glutamine plus P/S. 100 μl per well was added to a 96-well tissue culture plate (flat bottom), i.e. 2.0×10⁴ transfected cells per well, using a multi-channel pipette and the plate was then incubated overnight.

D) Immobilisation of Notch Ligand Protein Directly onto a 96-Well Tissue Culture Plate

10 μg of purified Notch ligand protein was added to sterile PBS in a sterile Eppendorf tube to give a final volume of 1 ml. Serial 1:2 dilutions were made by adding 500 μl into sterile Eppendorf tubes containing 500 μl of sterile PBS to generate dilutions of 10 μg/ml, 5 μg/ml, 2.5 μg/ml, 1.25 μg/ml, 0.625 jig/ml and 0 μg/ml.

The lid of the plate was sealed with parafilm and the plate was left at 4° C. overnight or at 37° C. for 2 hours. The protein was then removed and the plate was washed with 200 μl of PBS.

E) A20-Delta Cells

The IVS, IRES, Neo and pA elements were removed from plasmid pIRESneo2 (Clontech, USA) and inserted into a pUC cloning vector downstream of a chicken beta-actin promoter (eg see GenBank Accession No E02199). Mouse Delta-1 (eg see GenBank Accession No NM_{—}007865) was inserted between the actin promoter and IVS elements and a sequence with multiple stop codons in all three reading frames was inserted between the Delta and IVS elements.

The resulting construct was transfected into A20 cells using electroporation and G418 to provide A20 cells expressing mouse Delta1 on their surfaces (A20-Delta).

F) CHO and CHO-hDelta1-V5-His Assay Control

CHO cells were maintained in DMEM plus 10% (HI)FCS plus glutamine plus P/S and CHO-hDelta1-V5-His (clone#10) cells were maintained in DMEM plus 10% (HI)FCS plus glutamine plus P/S plus 0.5 mg/ml G418.

Cells were removed using 0.02% EDTA solution (Sigma), spun down and resuspended in 10 ml DMEM plus 10% (HI)FCS plus glutamine plus P/S. 10 μl of cells were counted and the cell density was adjusted to 5.0×10⁵ cells/ml with fresh DMEM plus 10% (HI)FCS plus glutamine plus P/S. 300 μl of each cell line at 5.0×10⁵ cells/ml was added into duplicate wells of a 96-well tissue culture plate. 150 μl of DMEM plus 10% (HI)FCS plus glutamine plus P/S was added in to the next 5 wells below each well. 150 μl of cells were serially diluted into the next 4 wells giving cell density dilution of 5.0×10⁵ cells/ml, 2.5×10⁵ cells/ml, 1.25×10⁵ cells/ml, 0.625×10⁵ cells/ml, 0.3125×10⁵ cells/ml and 0 cells/ml.

100 μl from each well was added into the 96-well plate containing 100 μl of CHO-N2 cells transfected with 10×CBF1-Luc (2.0×10⁴ transfected CHO-N2 cells/well) and the plate was left in an incubator overnight.

G) Cell Co-Culture

5×10⁴ CHO-N2 cells were plated on a 96 well plate. CHO-Delta or A20-Delta cells were titrated in as required (max ratio CHO-N2: CHO-Delta was 1:1, max ratio CHO-N2: A20-Delta was 1:2). The mixture was incubated overnight before conducting a luciferase assay.

H) Luciferase Assay

Supernatant was removed from all wells. 100 μl of PBS and 100 μl of SteadyGlo™ luciferase assay reagent (Promega) was added and the cells were left at room temperature for 5 minutes. The mixture was pipetted up and down 2 times to ensure cell lysis and contents from each well were transferred into a white 96-well OptiPlate™ (Packard). Luminescence was measured in a TopCount™ counter (Packard).

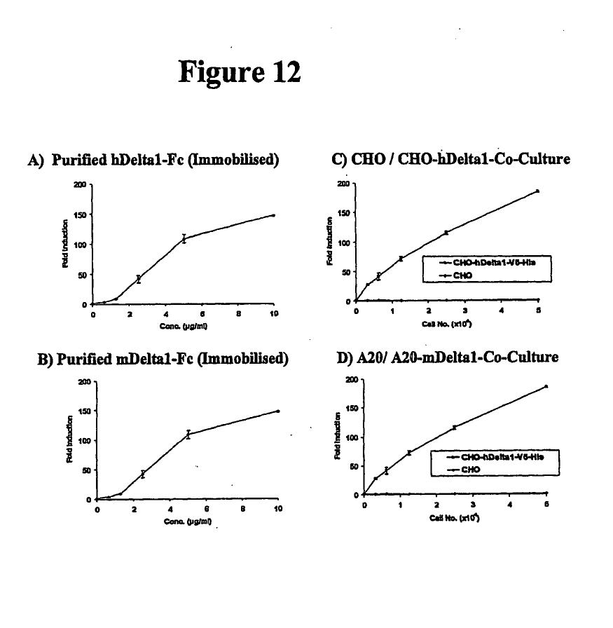

Results of sample assays (using the stable CHO-Notch2-10×CBF1-Luc reporter cell line described above with (A) plate-immobilised human Delta-1/Ig4Fc fusion protein, (B) plate-immobilised mouse Delta-1/Ig4Fc fusion protein, (C) CHO/CHO-human Delta1 co-cultured cells and (D) A20/A20-mouse Delta1 co-cultured cells as actives against corresponding controls) are shown in FIGS. 12A to D.

### Example 9

### Dynabeads Luciferase Assay Method For Detecting Notch Ligand Activity

Fc-tagged Notch ligands were immobilised on Streptavidin-Dynabeads (CELLection Biotin Binder Dynabeads [Cat. No. 115.21] at 4.0×10⁸ beads/ml from Dynal (UK) Ltd; beads) in combination with biotinylated α-IgG-4 (clone JDC14 at 0.5 mg/ml from Pharmingen [Cat. No. 555879]) as follows:

2.5×10⁷ beads (62.5 μl of beads at 4.0×10⁸ beads/ml) and 5 μg biotinylated α-IgG-4 was used for each sample assayed. PBS was added to the beads to 1 ml and the mixture was spun down at 13,000 rpm for 1 minute. Following washing with a further 1 ml of PBS the mixture was spun down again. The beads were then resuspended in a final volume of 100 μl of PBS containing the biotinylated α-IgG-4 in a sterile Eppendorf tube and placed on shaker at room temperature for 30 minutes. PBS to was added to 1 ml and the mixture was spun down at 13,000 rpm for 1 minute and then washed twice more with 1 ml of PBS.

The mixture was then spun down at 13,000 rpm for 1 minute and the beads were resupsended in a 50 μl PBS per sample. 50 μl of biotinylated α-IgG-4-coated beads were added to each sample and the mixture was incubated on a rotary shaker at 4° C. overnight. The tube was then spun at 1000 rpm for 5 minutes at room temperature.

The beads then were washed with 10 ml of PBS, spun down, resupended in 1 ml of PBS, transferred to a sterile Eppendorf tube, washed with a further 2×1 ml of PBS, spun down and resuspended beads in a final volume of 250 μl of DMEM plus 10% (HI)FCS plus glutamine plus P/S, i.e. at 1.0×10⁵ beads/μl.

Stable N27#11 cells from Example 8 (T₈₀ flask)were removed using 0.02% EDTA solution (Sigma), spun down and resuspended in 10 ml DMEM plus 10% (HI)FCS plus glutamine plus P/S. 10 μl of cells were counted and the cell density was adjusted to 1.0×10⁵ cells/ml with fresh DMEM plus 10% (HI)FCS plus glutamine plus P/S. 1.0×10⁵ of the cells were plated out per well of a 24-well plate in a 1 ml volume of DMEM plus 10% (HI)FCS plus glutamine plus P/S and cells were placed in an incubator to settle down for at least 30 minutes.

100 μl of beads were then added in duplicate to the first pair of wells to give 1.0×10⁷ beads/well (100 beads/cell); 20 μl of beads added in duplicate to the second pair of wells to give 2.0×10⁶ beads/well (20 beads/cell); 4 μl of beads added in duplicate to the third pair of wells to give 4.0×10⁵ beads/well (4 beads/cell) and 0 μl of beads added to the fourth pair of wells. The plate was left in a CO₂ incubator overnight.

Luciferase Assay

Supernatant was then removed from all the wells, 150 μl of PBS and 150 μl of SteadyGlo luciferase assay reagent (Promega) were added and the resulting mixture left at room temperature for 5 minutes.

The mixture was then pipetted up and down 2 times to ensure cell lysis and the contents from each well were transferred into an Eppendorf tube, spun at 13,000 rpm for 1 minute and the cleared supernatant was transferred to a white 96-well OptiPlate™ (Packard), leaving the bead pellet behind. Luminescence was then read in a TopCount™ (Packard) counter.

### Example 10

### Dynabeads ELISA Assay Method For Detecting Notch Ligand Activity

M450 Streptavidin Dynabeads were coated with anti-hamster-IgG1 biotinylated monoclonal antibody, anti-human-IgG4 biotinylated monoclonal antibody or both antibodies and rotated for 2 hours at room temperature.

Beads were washed three times with PBS (1 ml). The anti-hamster-IgG1 beads were then further incubated with anti-CD3ε chain monoclonal antibody, the anti-human-IgG4 beads were further incubated with Fc-Delta, and the double coated beads incubated with both anti-CD3ε chain monoclonal antibody and Fc-Delta. Beads were rotated overnight at 4° C., washed three times with PBS (1 ml) and resuspended.

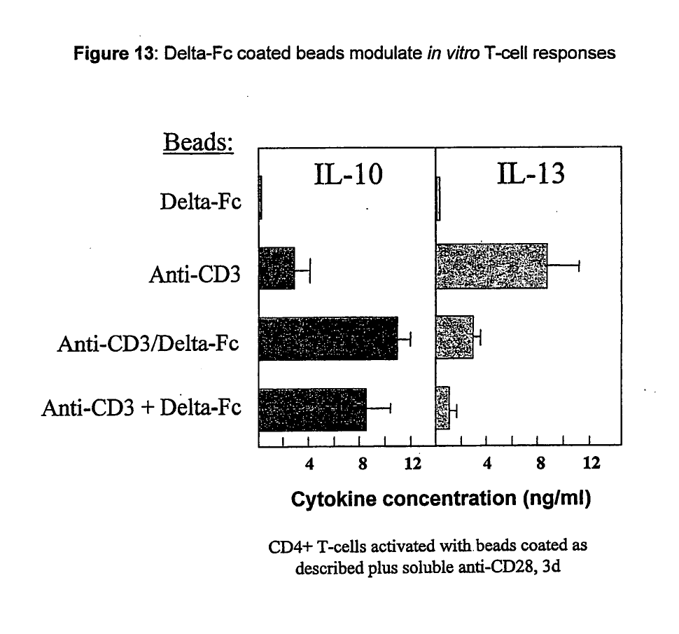

T-cell assays were carried out with CD4+ T-cells and the beads. Supernatants were removed after 72 hours and cytokines measured by ELISA as described in Example 3. Results are shown in FIG. 13.

### Example 11

### Modulation of Cytokine Production by Human CD4+ T Cells in the Presence of Delta1-hIgG4 Immobilised on Dynal Microbeads

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

Human CD4+ T cells were isolated by positive selection using anti-CD4 microbeads from Miltenyi Biotech according to the manufacturer's instructions.

The CD4+ T cells were incubated in triplicates in a 96-well-plate (flat bottom) at 10⁵ CD4/well/200 μl in RPMI medium containing 10% FCS, glutamine, penicillin, streptomycin and β₂-mercaptoethanol.

Cytokine production was induced by stimulating the cells with anti-CD3/CD28 T cell expander beads from Dynal at a 1:1 ratio (bead/cell) or plate bound anti-CD3 (clone UCHT1, BD Biosciences, 5 μg/ml) and soluble anti-CD28 (clone CD28.2, BD Biosciences, 2 μg/ml). Beads coated with mouse Delta1EC domain-hIgG4 fusion protein (prepared as described above with the modifications that incubation with human IgG4 was for 30-40 minutes at room temperature and incubation with Delta-Fc was for two hours at room temperature) or control beads were added in some of the wells at a 10:1 ratio (beads/cell). The supernatants were removed after 3 or 4 days of incubation at 37° C./5% CO₂/humidified atmosphere and cytokine production was evaluated by ELISA using Pharmingen kits OptEIA Set human IL10 (catalog No. 555157), OptEIA Set human IL-5 (catalog No. 555202) and OptEIA Set human IFNg (catalog No 555142) for IL-10, IL-5 and IFNg respectively and a human TNFa DuoSet from R&D Systems (catalog. No. DY210) for TNFa according to the manufacturer's instructions.

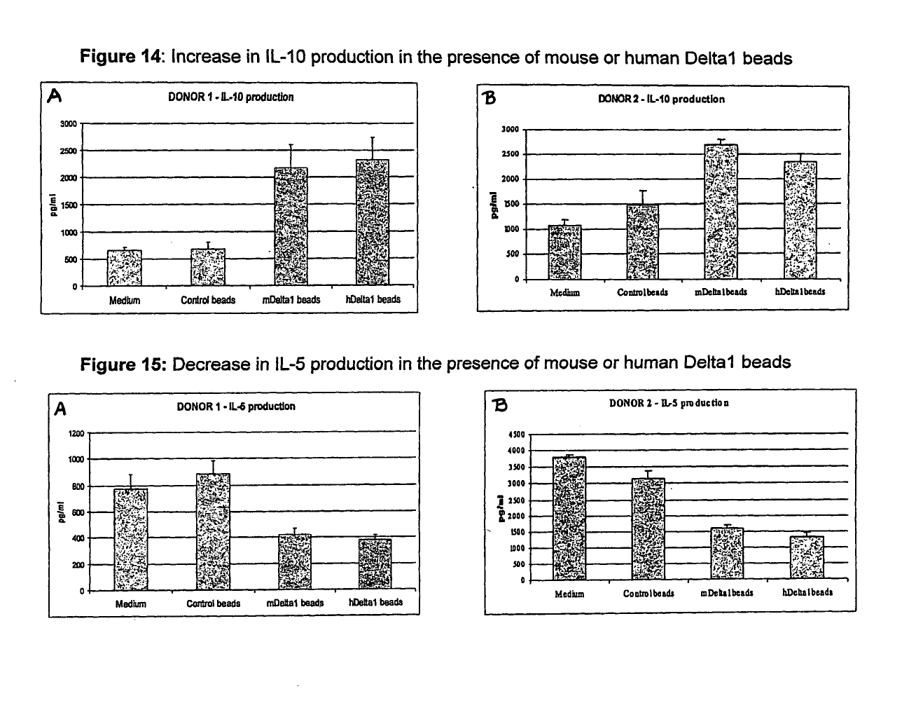

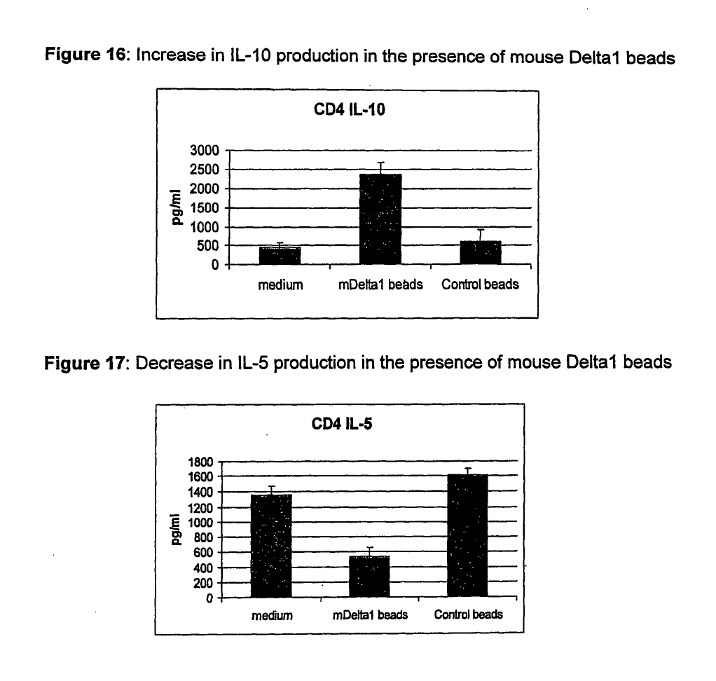

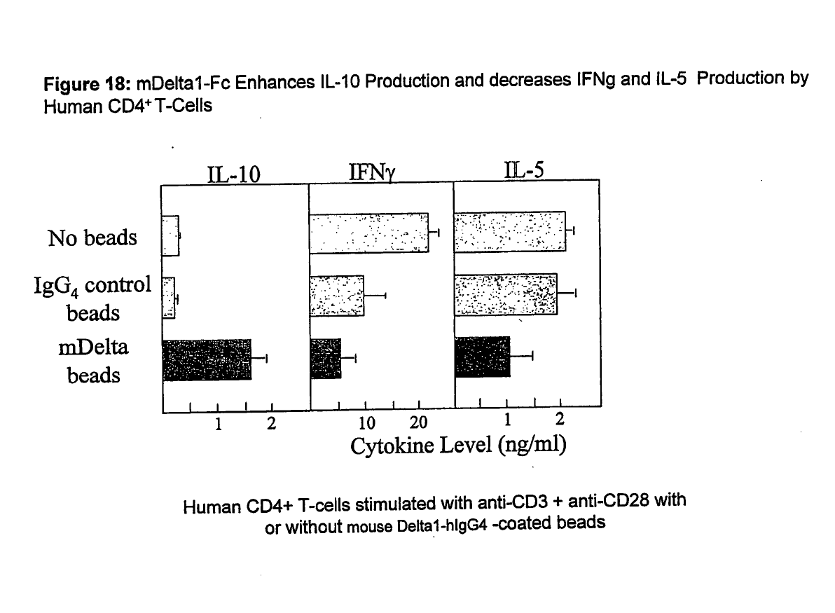

Results are shown in FIGS. 14 to 18.

### Example 12

### Variation of Bead:Cell Ratios

The procedure of Example 11 was repeated except that the ratio of control beads to cells and mouse Delta1-hIgG4 fusion protein coated beads to cells was varied between 16:1 and 0.25:1 (variously 16:1, 8:1, 4:1, 2:1, 1:1, 0.5:1, 0.25:1) and human Delta1-hIgG4 fusion protein coated beads were also used at the same ratios for comparison.

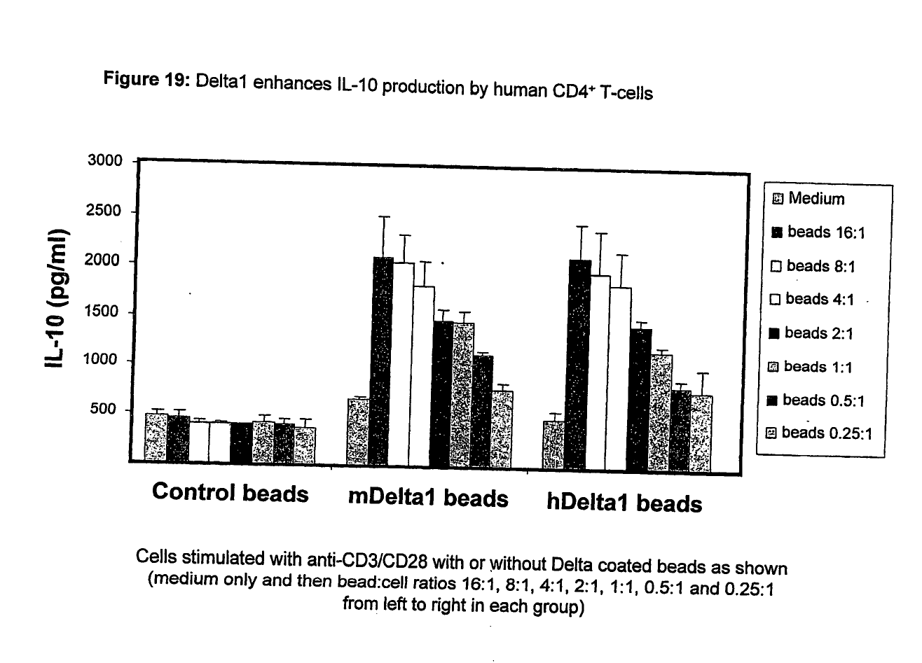

Results are shown in FIG. 19.

### Example 13

### Comparison of CD45RO+ (Memory Cells) and CD45RO− (Naive Cells)

The procedure of Example 11 was repeated except that prior to the stimulation the human CD4+ were separated into CD45RO+ (memory cells) and CD45RO− (naive cells, data not shown on the slide). The magnetic separation was done using anti-CD4 Multisort microbeads (cat.No. 551-01) and then anti-CD45RO microbeads (cat.No.460-01) supplied by Miltenyi Biotech and following Miltenyi's protocol.

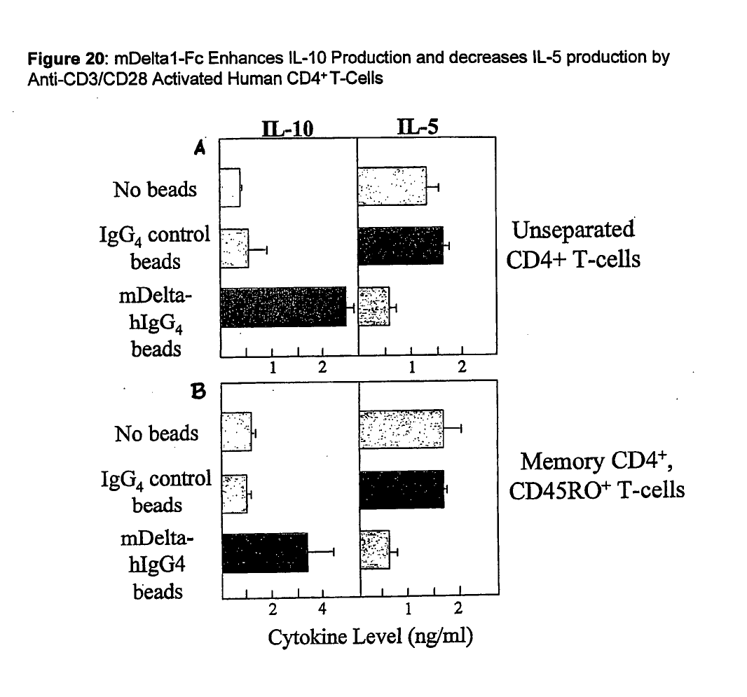

Results are shown in FIG. 20.

### Example 14

### Measurement of Cytokine Production in Stimulated Mouse CD4+ Cells Under Polarising Conditions

(i) CD4+ Cell Purification

Spleens were removed from mice (variously Balb/c females, 8-10 weeks, C57B/6 females, 8-10 weeks, CARD1 females, 8-10 weeks (D011.10 transgenic, CAR transgenic)) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHC0₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

96 well flat-bottomed plates were coated with Dulbecco's Phosphate Buffered Saline (DPBS) plus 1 μg/ml anti-CD3 antibody (Pharmingen, San Diego, US: Cat No 553058, Clone No 145-2C11) plus 1 μg/ml anti-IgG4 antibody (Pharmingen Cat No 555878). 100 μl of coating mixture was used per well. Plates were incubated overnight at 4° C. then washed with DPBS. Each well then received either 100 μl DPBS or 100 μl DPBS plus 10 μg/ml Notch ligand (mouse Delta 1 extracellular domain/Ig4Fc fusion protein; Fc-delta). The plates were incubated for 2-3 hours at 37° C. then washed again with DPBS before cells (prepared as in (i)) were added.

(iii) Primary Polyclonal Stimulation

CD4+ cells were cultured in 96 well, flat-bottomed plates pre-coated as in (ii) above. Cells were re-suspended, following counting, at 2×10⁶/ml in R10F medium plus 4 μg/ml anti-CD28 antibody (Pharmingen, Cat No 553294, Clone No 37.51). 100 82 l cell suspension was added per well. 100 μl of polarising or control medium was then added to each well to give a final volume of 200 μl (2×10⁵ cells/well, anti-CD28 final concentration 2 μg/ml) as follows:

Un-polarised cells: R10F medium.

Th1 polarised cells: R10F medium plus anti-IL-4 antibody (10 μg/ml, Pharmingen Cat No 554432) plus IL-12 (10 ng/ml, Peprotech 210-12).

Th2 polarised cells: R10F medium plus anti-IL-12 antibody (10 μg/ml, Pharmingen Cat No 554475) plus anti-IFNg antibody (1 μg/ml, Pharmingen Cat No 554408) plus IL-4 (10 ng/ml, Peprotech Cat No 214-14).

The plates were then incubated at 37° C. for 72 hours.

125 μl supernatant was then removed from each well and stored at −20° C. until tested by ELISA for IL-10 and TNFa using antibody pairs from R & D Systems (Abingdon, UK). The cells were then split 1 in 3 into new wells (not coated) and fed with R10F medium plus recombinant human IL-2 (2.5 ng/ml, PeproTech Inc, London, UK: Cat No 200-02).

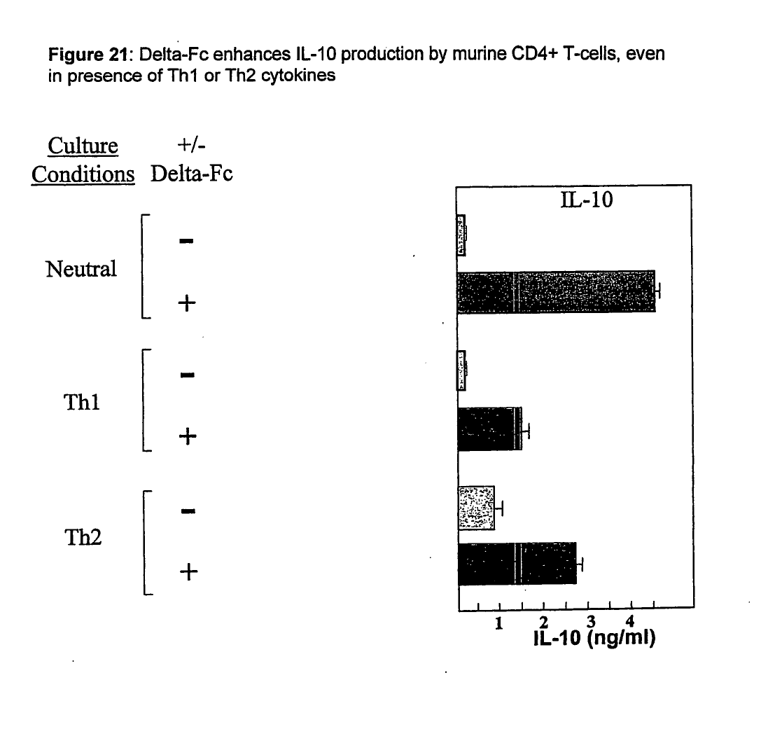

Results are shown in FIG. 21.

### Example 15

### Gene Expression Profiling

(i) Cell Culture, Treatments and RNA Extraction

Jurkat cells were cultured in RPMI 1640 (GibcoBRL) supplemented with 2 mM Glutamine (GibcoBRL), Penicillin-Streptomycin 50 units/ml (GibcoBRL) and with 10% Fetal Bovine Serum (FBS) (Biochrom KG).

Anti-V5 (Invitrogen) and anti-CD3 (human), anti-CD28 (human) antibodies (PharMingen) were plated at 5 μg/ml in phosphate buffer saline (Gibco BRL) in 6 well tissue culture dishes (1 ml PBS/well) overnight. Anti-V5 antibody was applied to every well, while mouse IgG_{I}κ isotype control at 10 μg/ml was applied in wells that no anti-CD3 or anti-CD28 was used. The next day the wells were washed 3 times with PBS, and Delta-V5-His protein was plated at 5 μg/ml PBS (1 ml/well). The plates were then incubated at 37° C. for 2 hours and then washed with PBS three times. Jurkat cells were then plated out at a concentration of 2×10⁶ cells/ml and incubated at 37° C. Ionomycin was added to the appropriate wells at a concentration of 1 μg/ml (Sigma). Cells were taken out at 2, 4, 8, 18, 24, 36, 48 hrs, washed once with PBS at 4° C. and collected at 300-600 μl RLT lysis solution (Qiagen). In order to ensure the efficacy of the stimulation, cells were tested for the correct expression of T cell activation markers using FACs analysis. The cells used in this experiment were all expressing CD69 (early activation marker) after 48 h of anti-CD3, anti-CD28 activation.

RNA was extracted using an RNA Easy miniprep kit (Qiagen) according to the manufacturer's instructions. The optional DNase step recommended was also performed. A phenol extraction step was performed to ensure the complete lack of proteins in the RNA. RNA was then amplified using the MessageAmp aRNA Kit (Ambion) following the manufacturer's recommendations. Briefly, the procedure consists of reverse transcription with an oligo(dT) primer bearing a T7 promoter and in vitro transcription of the resulting DNA with T7 RNA polymerase to generate hundreds of thousands of antisense RNA (αRNA) copies of each mRNA in the sample.

The nomenclature used was as follows: RNA from cells that were plated on wells treated only with V5 was labelled ‘V5’, while RNA from cells plated on wells treated with anti-V5 and Delta-V5-His was labelled ‘Delta’. RNA from cells plated on wells treated with anti-V5, anti-CD3, anti-CD28 were labelled ‘CD3CD28’ while RNA from cells plated on wells treated with anti-V5, anti-CD3, anti-CD28, Delta-V5-His was labelled ‘CD3CD28Delta’. Similarly RNA from cells plated on anti-V5 and further treated with ionomycin was labelled ‘ionomycin’ while RNA from cells plated on anti-V5, Delta-V5-His and further treated with ionomycin were labelled ‘ionomycin-Delta’.

(ii) Gene Expression Profiling

Microarrays were manufactured by spotting purified PCR products onto glass slides. Microarray probes were prepared by labelling 2 μg of αRNA by a reverse transcriptase reaction incorporating dCTP-Cy3 or dCTP-Cy5 labelled nucleotide. Probe labelling and purification were then performed generally as described in Hegde P, Qi R, Abernathy K, Gay C, Dharap S, Gaspard R, Hughes J E, Snesrud E, Lee N, Quackenbush J: A concise guide to cDNA microarray analysis (2000). Biotechniques 29:548-50, 552-4, 556 passim.

Purified probes were then hybridized on the arrays overnight at 42° C. in 10×SSC, 50% formamide, 0.2% SDS solution. Slides were then washed twice in 2×SSC, 0.2% SDS for 7 min at 42° C., twice in 0.1 SSC/0.2% SDS for 5 minutes at room temperature, and finally once in 0.2% SSC at room temperature. For each time point the sample named ‘V5’ was labelled with dCTP-Cy3 and hybridized on the same slide as the sample named ‘Delta’ that was labelled with dCTP-Cy5. Similarly the sample named CD3CD28 was labelled with dCTP-Cy3 and hybridized on the same slide as the sample named ‘CD3CD28Delta’ that was labelled with dCTP-Cy5. Finally the sample named ‘ionomycin’ was labelled with dCTP-Cy3 and hybridized on the same slide as the sample labelled ‘ionomycinDelta’ that was labelled with dCTP-Cy5 (see Table-1).

TABLE 1

| | Label 1 (Cy3-dCTP) | Label 2 (Cy5-dCTP) |
|---|---|---|
| Slide | V5 | Delta |
| Slide | CD3CD28 | CD3CD28Delta |
| Slide | Ionomycin | IonomycinDelta |

Once dried the slides were scanned on a GSI Lumonics confocal scanner at 100% laser power and 65-75% photo-multiplier tube efficiency (depending on background). Slide images were processed as follows: Array spots representing the signal associated with individual spotted clones were identified and quantified using the Quantarray application (GSI Lumonics). Numeric values for the gene expression intensities were calculated using the histogram method implemented in the same application. Values were calculated as integrals of the pixel signal distribution associated to each spot and local background values subtracted (raw data).

(iii) Data Processing

For all data analyses the GeneSpring package (Silicon Genetics) was used. Raw data from Quantarray was introduced in GeneSpring, and the ratio between the signal and control intensities was calculated for each gene at each time point. Intensities for genes from samples labelled ‘Delta’ or ‘CD3CD28Delta’, or ‘ionomycinDelta’ were regarded as ‘signals’ while the intensities from genes from samples labelled either ‘V5’ or ‘CD3CD28’ or ‘ionomycin’ were regarded as ‘controls’.
Ratio=signal strength of gene in ‘Delta’/control strength of gene in ‘V5’
Ratio=signal strength of gene in ‘CD3CD28Delta’/control strength of gene in ‘CD3CD28’
Ratio=signal strength of gene in ‘ionomycinDelta’/control strength of gene in ‘ionomycin’

When this ratio was >2 the gene was considered to be upregulated, while when the ratio was <0.5 the ratio the gene was considered to be downregulated.

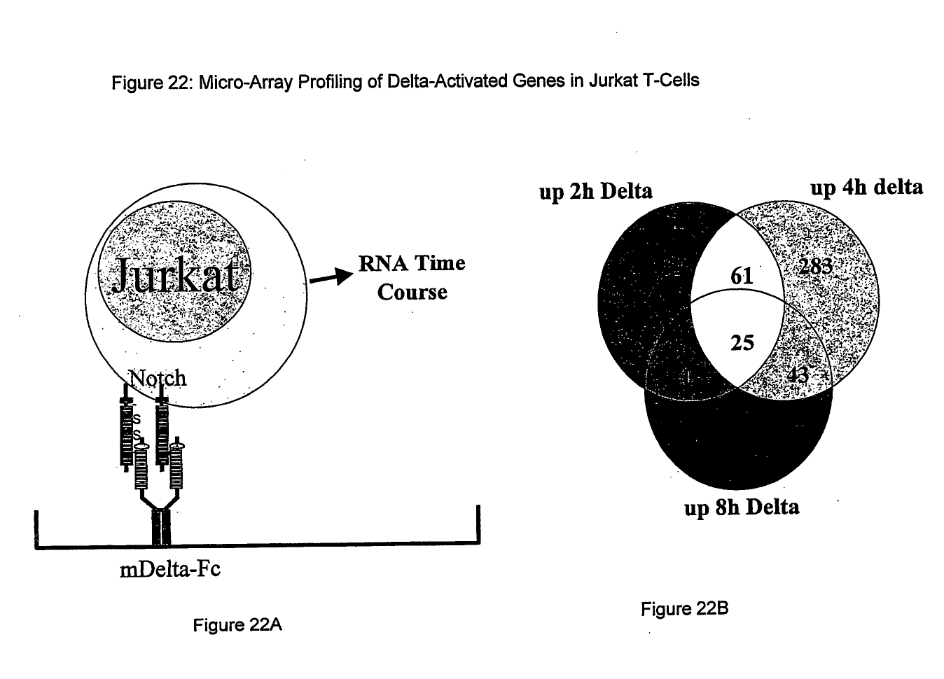

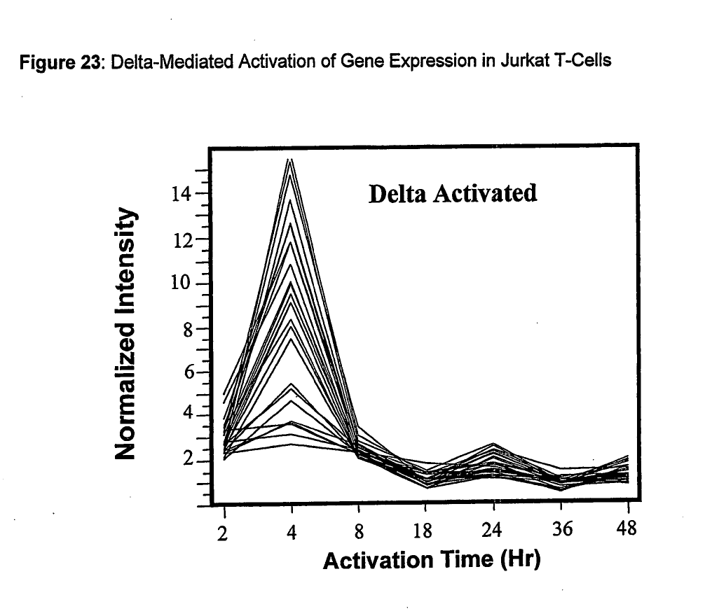

A schematic representation of the protocol for activating with Delta alone and a Venn diagram showing numbers of genes showing increased expression in response to Delta activation alone are shown in FIGS. 22A and 22B respectively, and a corresponding time-course expression profile is shown in FIG. 23.

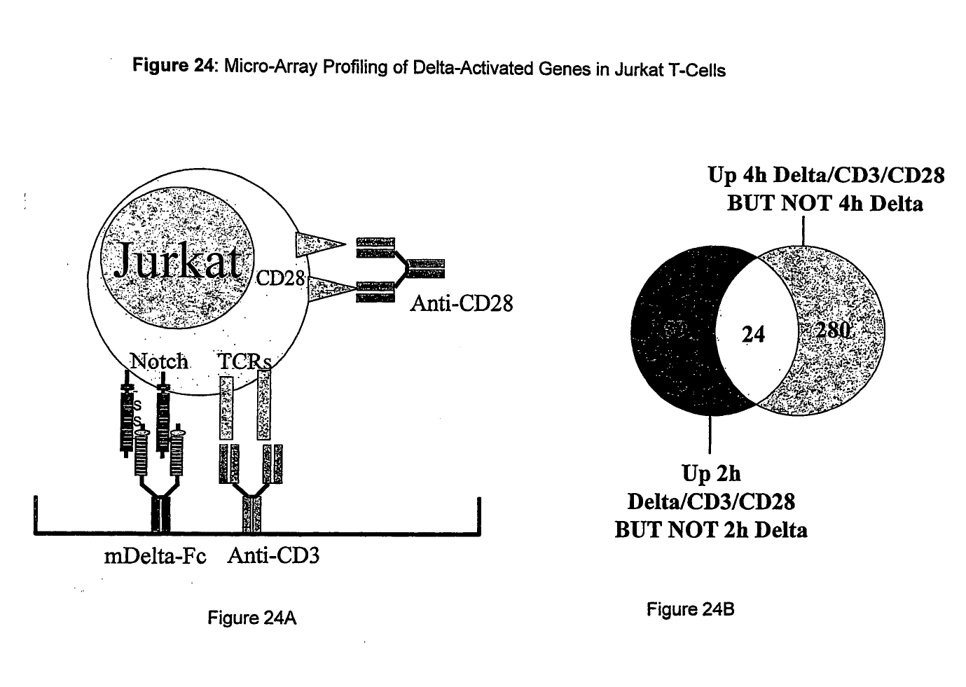

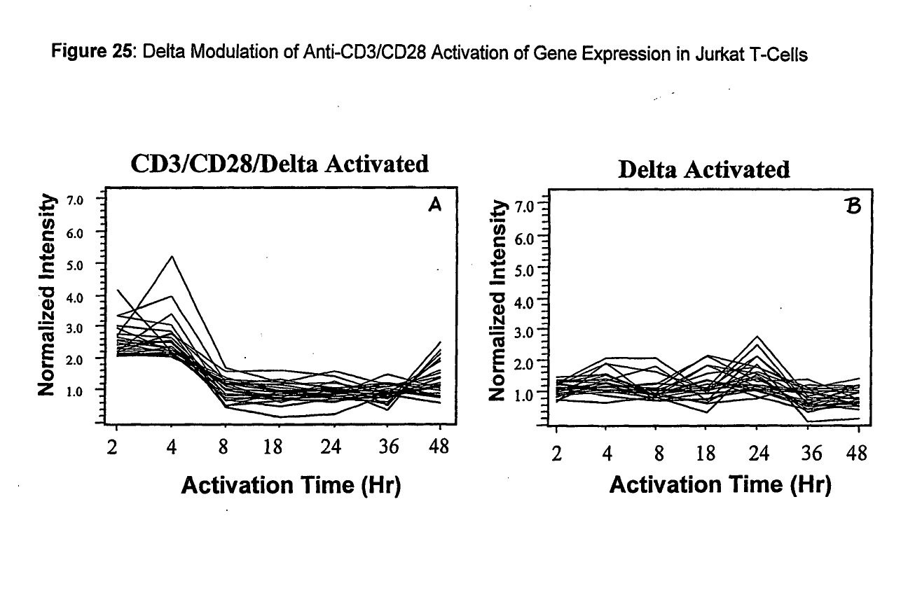

A schematic representation of the protocol for activating with both Delta and anti-CD3/CD28 activation and a Venn diagram showing numbers of genes showing increased expression in response to Delta activation in combination with anti-CD3/CD28 activation but not Delta activation alone are shown in FIGS. 24A and 24B respectively, and a corresponding time-course expression profile is shown in FIG. 25.

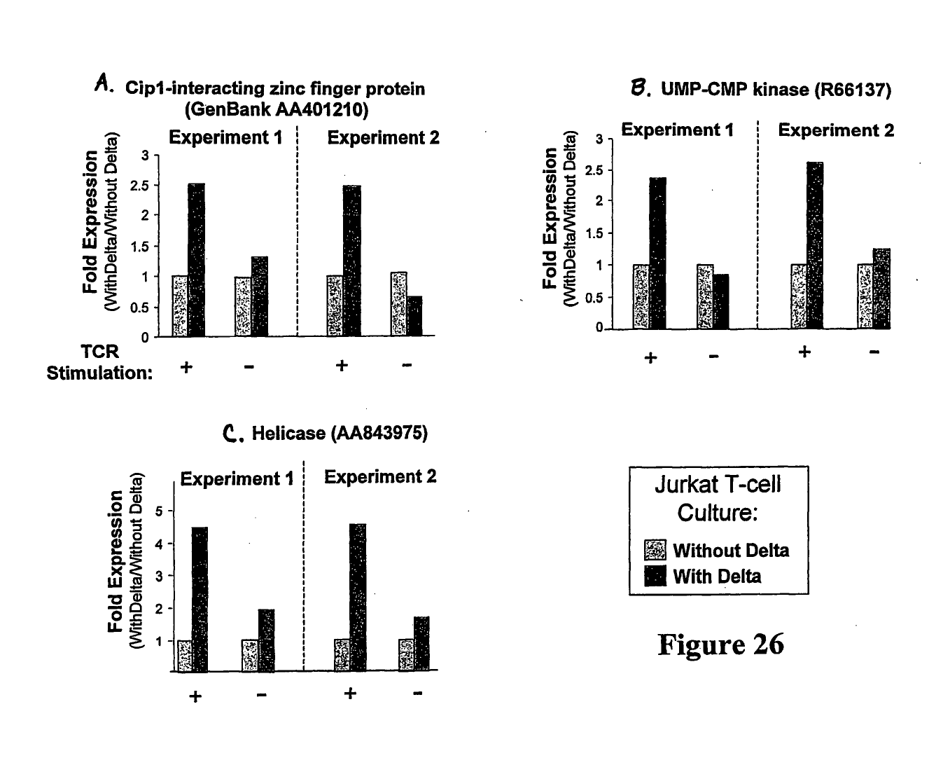

Some specific genes showing increased expression in response to Delta activation in combination with anti-CD3/CD28 activation in comparison with Delta activation alone are shown in FIG. 26.

### Example 16

### Reporter Assay Using Jurkat Cell Line

As Jurkat cells cannot be cloned by simple limiting dilution a methylcellulose-containing medium (ClonaCell™ TCS) was used with these cells.

Jurkat E6.1 cells (lymphoblast cell line; ATCC No TIB-152) were cloned using ClonaCell™ Transfected Cell Selection (TCS) medium (StemCell Technologies, Vancouver, Canada and Meylan, France) according to the manufacturer's guidelines.

Plasmid pLOR92 (prepared as described above) was electroporated into the Jurkat E6.1 cells with a Biorad Gene Pulser II electroporator as follows:

Actively dividing cells were spun down and resuspended in ice-cold RPMI medium containing 10% heat-inactivated FCS plus glutamine plus penicillin/streptomycin (complete RPMI) at 2.0×10⁷ cells per ml. After 10 min on ice, 0.5 ml of cells (ie 1×10⁷ cells) was placed into a pre-cooled 4 mm electroporation cuvette containing 20 μg of plasmid DNA (Endo-free Maxiprep DNA dissolved in sterile water). The cells were electroporated at 300 v and 950 μF and then quickly removed into 0.5 ml of warmed complete RPMI medium in an Eppendorf tube. The cells were spun for at 3000 rpm for 1 min in a microfuge and placed at 37° C. for 15 min to recover from being electroporated. The supernatant was then removed and the cells were plated out into a well of a 6-well dish in 4 ml of complete RPMI and left at 37° C. for 48 h to allow for expression of the antibiotic resistance marker.

After 48 h the cells were spun down and resupended in to 10 ml fresh complete RPMI. This was then divided into 10×15 ml Falcon tubes and 8 ml of pre-warmed ClonaCell-TCS medium was added followed by 1 ml of a 10×final concentration of the antibiotic being used for selection. For G418 selection the final concentration of G418 was 1 mg/ml so a 10 mg/ml solution in RPMI was prepared and 1 ml of this was added to each tube. The tubes were mixed well by inversion and allowed to settle for 15 min at room temperature before being plated out into 10 cm tissue culture dishes. These were then placed in a CO2 incubator for 14 days when that were examined for visible colonies.

Macroscopically visible colonies were picked off the plates and these colonies were expanded through 96-well plates to 24-well plates to T25 flasks.

The resulting clones were each transiently transfected with pLOR91 using Lipofectamine 2000 reagent and then plated out onto a 96-well plate containing plate-bound immobilised hDLL1-Fc (prepared as described above). Four well-performing clones were selected and used for further study.

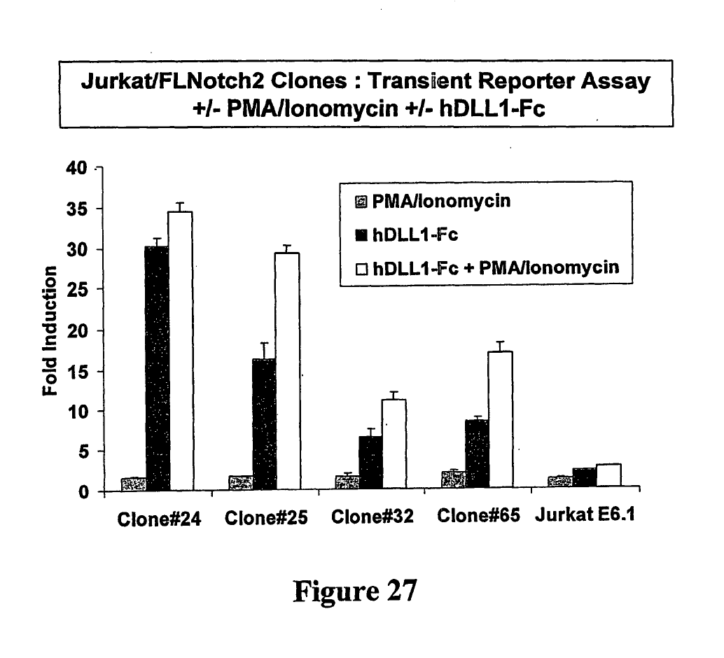

Luciferase assays were then conducted with each of the four clones with or without plate-bound immobilised hDLL1-Fc and with or without PMA/ionomycin (both from Sigma) at 50 ng/ml PMA plus 1 μg/ml ionomycin final concentration. Results are shown in FIG. 27 (with results from native Jurkat E6.1 cells also shown for comparison).

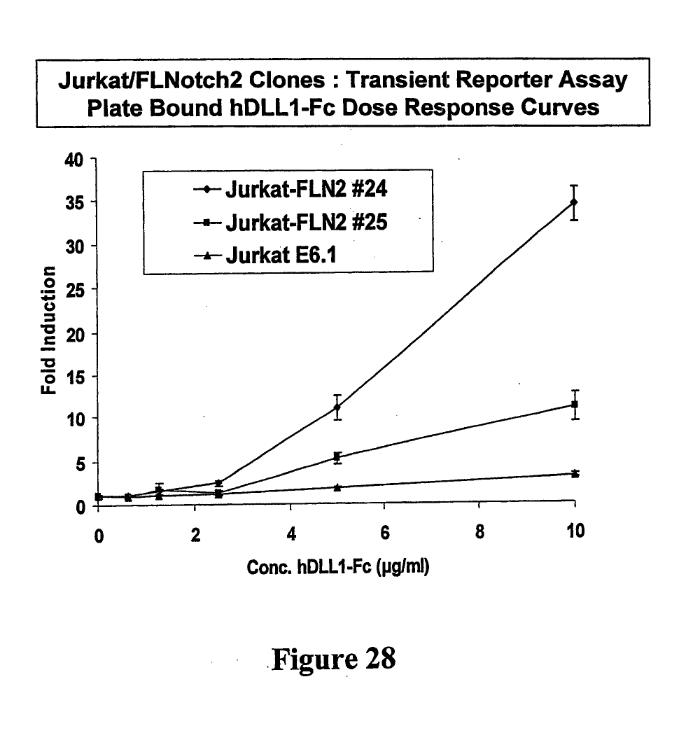

FIG. 28 shows a dose response to plate-bound hDLL1-Fc with two selected clones with results from native Jurkat E6.1 cells also shown for comparison.

### Example 17

### Reporter Assay with Variation of Ionomycin Concentration

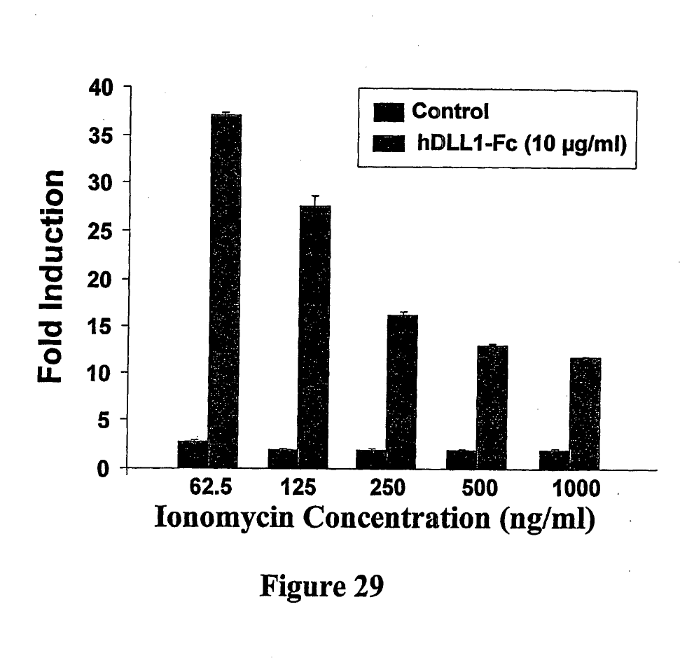

The procedure of Example 16 was repeated with ionomycin concentrations of 1000, 500, 250, 125 and 62.5 ng/ml and controls. Results are shown in FIG. 29.

### Example 18

### Reporter Assay with Notch Signalling by Notch IC

Notch IC Construct

Human Notch1 intracellular domain (NIC1) was cloned into the expression vector pcDNA3.1 (Invitrogen, Carlsbad, Calif., USA and Paisley, UK) as a NotI/EcoRI fragment.

Human Notch1-IC was cloned as follows:

A human placental arrayed cDNA library (Origene) was screened by PCR using the following pair of primers specific for the intracellular domain of human Notch1:

hN1F:
CAC CCC ATG GCT ACC TGT CAG
hN1R:
GGC TGC ACC TGC TGG GTC TGC

The PCR was carried out on an MJ Tetrad PCR machine using HotStar Taq polymerase (Qiagen) and the following cycle parameters:
95° C., 15′
94° C., 30 s
65° C., 30 s
72° C., 45 s
30 cycles of these last three steps, followed by
72° C., 10′
16° C., soak

Under these conditions, the primers generate a specific diagnostic product of 500 bp from a human Notch1 cDNA target. Using this PCR screening protocol, a positive human Notch1 clone (#3) was identified and sequenced to confirm its identity. Subsequently, the intracellular domain was amplified from #3 using the following primers:

hN1-IC1759:
AAA GGA TTC ACC ATG GCA CGC AAG CGC CGG CGC AGT
CAT (contains initiation methionine in bold)
hN1-IC 2556:
GCG CTC GAG TTA CTT GAA CGC CTC CGG GAT GCG
(contains stop codon in italics)

The PCR was carried out on an MJ Tetrad PCR machine using Pfu DNA polymerase (Stratagene) and the following cycle parameters:
94° C., 2′
94° C., 45 s
58° C., 45 s
72° C., 3′
20 cycles of these last three steps, followed by
72° C., 10′
16° C., soak

This generated a specific product of approximately 2.6 kb corressponding to the intracellular domain of human Notch1. The PCR product was digested with BamHI and XhoI (these sites are present within the amplimers) and cloned into the mammalian expression vector pcDNA3.1 (Invitrogen) using the BamHI and XhoI sites present within the multiple cloning site of this vector. The sequence of the hNotch1-IC was confirmed by sequencing, and the protein sequence encoded by this cloned sequence is as follows:

MARKRRRQHGQLWFPEGFKVSEASKKKRREPLGEDSVGLKPLKNASDGAL
MDDNQNEWGDEDLETKKFRFEEPVVLPDLDDQTDHRQWTQQHLDAADLRM
SAMAPTPPQGEVDADCMDVNVRGPDGFTPLMIASCSGGGLETGNSEEEED
APAVISDFIYQGASLHNQTDRTGETALHLAARYSRSDAAKRLLEASADAN
IQDNMGRTPLHAAVSADAQGVFQILIRNRATDLDARMHDGTTPLILAARL
AVEGMLEDLINSHADVNAVDDLGKSALHWAAAVNNVDAAVVLLKNGANKD
MQNNREETPLFLAAREGSYETAKVLLDHFANRDITDHMDRLPRDIAQERM
HHDIVRLLDEYNLVRSPQLHGAPLGGTPTLSPPLCSPNGYLGSLKPGVQG
KKVRKPSSKGLACGSKEAKDLKARRKKSQDGKGCLLDSSGMLSPVDSLES
PHGYLSDVASPPLLPSPFQQSPSVPLNHLPGMPDTHLGIGHLNVAAKPEM
AALGGGGRLAFETGPPRLSHLPVASGTSTVLGSSSGGALNFTVGGSTSLN
GQCEWLSRLQSGMVPNQYNPLRGSVAPGPLSTQAPSLQHGMVGPLHSSLA
ASALSQMMSYQGLPSTRLATQPHLVQTQQVQPQNLQMQQQNLQPANIQQQ
QSLQPPPPPPQPHLGVSSAASGHLGRSFLSGEPSQADVQPLGPSSLAVHT
ILPQESPALPTSLPSSLVPPVTAAQFLTPPSQHSYSSPVDNTPSHQLQVP
EHPFLTPSPESPDQWSSSSPHSNVSDWSEGVSSPPTSMQSQIARIPEAFK

The Met and Ala residues at the 5′ end of the sequence are not endogenous residues but were incorporated, in the case of the Met, to form an initiation sequence, and for ease of cloning in the case of the Ala.

Jurkat Transfection

Jurkat E6.1 cells were routinely cultured in RPMI media supplemented with 10% foetal calf serum, glutamine and penicillin/streptomycin.

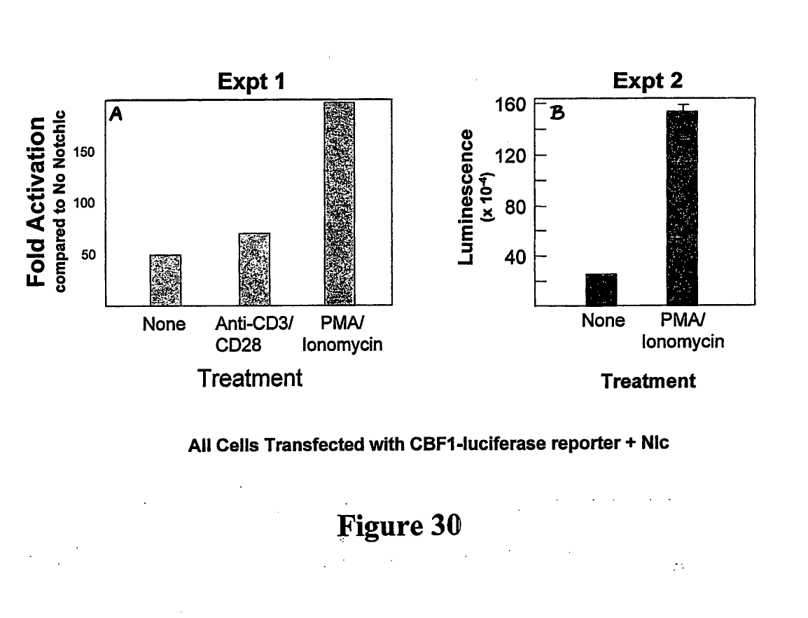

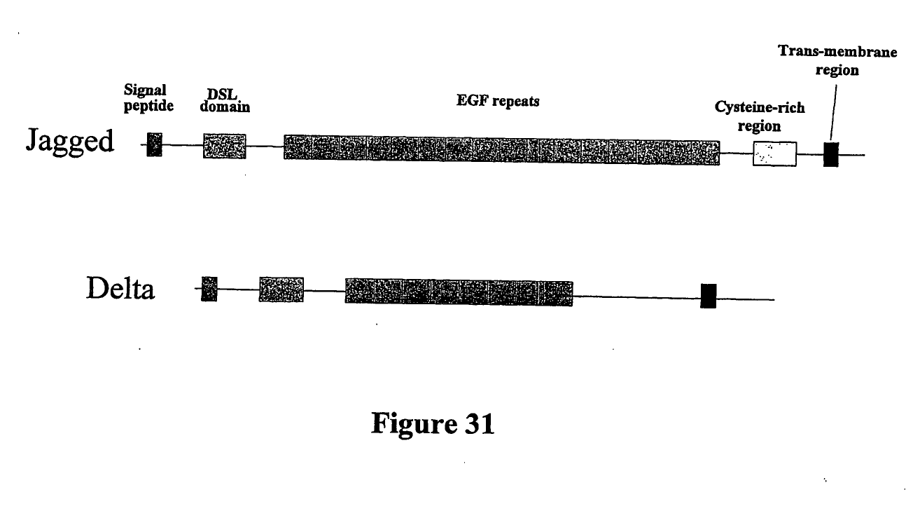

The cells were transfected with constructs (pLOR91 from Example 8 above and the NIC1 construct as described above) by electroporation in cold media in a 0.5 ml volume at 950 μF and 300V. After transfection, the cells were rapidly transferred to warm media and gently pelleted by centrifugation (1000 rpm, 30 seconds). The cells were then incubated as pellets for 20 minutes in an incubator before being plated out into 6 mls of fresh media in a 6-well dish. The cells were then incubated overnight, then washed, counted and plated out at approximately 150,000 cells per well in flat-bottomed 96-well plates ± stimulation with 50 ng/ml PMA; 500 ng/ml ionomycin; anti-human CD3 at 5 μg/ml, anti-human CD28 at 1 μg/ml. The cells were then incubated again overnight before being assayed for luciferase activity generally as described above (SteadyGlo from Promega) and read on a Hewlett-Packard TopCount luminometer. Results are shown in FIG. 30.

The invention is further described by the following numbered paragraphs:
1. A method for detecting modulators of Notch or immune signalling comprising the steps of (in any order):
   (a) activating Notch signalling in a cell of the immune system;
   (b) contacting the cell with a candidate modulator of Notch or immune signalling;
   (c) monitoring Notch or immune signalling; and
   (d) determining whether the candidate modulator modulates Notch or immune signalling.
2. A method for detecting modulators of Notch or immune signalling comprising the steps of (in any order):
   (a) activating a cell of the immune system;
   (b) contacting the cell with a candidate modulator of Notch or immune signalling;
   (c) monitoring Notch or immune signalling; and
   (d) determining whether the candidate modulator modulates Notch or immune signalling.
3. A method for detecting modulators of Notch or immune signalling comprising the steps of (in any order):
   (a) activating a cell of the immune system;
   (b) activating Notch signalling in the cell;
   (c) contacting the cell with a candidate modulator of Notch or immune signalling;
   (d) monitoring Notch or immune signalling; and
   (e) determining whether the candidate modulator modulates Notch or immune signalling.
4. A method for detecting modulators of Notch signalling comprising the steps of (in any order):
   (a) activating Notch signalling in a cell of the immune system;
   (b) contacting the cell with a candidate modulator of Notch signalling;
   (c) monitoring Notch or immune signalling; and
   (d) determining whether the candidate modulator modulates Notch or immune signalling.
5. A method for detecting modulators of Notch signalling comprising the steps of (in any order):
   (a) activating a cell of the immune system;
   (b) contacting the cell with a candidate modulator of Notch signalling;
   (c) monitoring Notch or immune signalling; and
   (d) determining whether the candidate modulator modulates Notch or immune signalling.
6. A method for detecting modulators of Notch signalling comprising the steps of (in any order):
   (a) activating a cell of the immune system;
   (b) activating Notch signalling in the cell;
   (c) contacting the cell with a candidate modulator of Notch signalling;
   (d) monitoring Notch or immune signalling; and
   (e) determining whether the candidate modulator modulates Notch or immune signalling.
7. A method according to any one of paragraphs 1 to 3 comprising the step of contacting the cell with a candidate modulator of Notch signalling.
8. A method according to any one of the preceding paragraphs comprising the step of monitoring Notch signalling.
9. A method according to any one of the preceding paragraphs comprising the step of determining whether the candidate modulator modulates Notch signalling.
10. A method according to any one of the preceding paragraphs comprising providing immune cell activation which is at least 20% optimal with respect to Notch or immune signalling.
11. A method according to any one of the preceding paragraphs comprising providing immune cell activation which is at least 70% optimal with respect to Notch or immune signalling.
12. A method according to any one of the preceding paragraphs wherein the candidate modulator is selected from the group consisting of: an organic compound, a inorganic compound, a peptide or polypeptide, a polynucleotide, an antibody, a fragment of an antibody, a cytokine and a fragment of a cytokine.
13. A method according to any one of the preceding paragraphs wherein the step of monitoring Notch signalling comprises the step of monitoring levels of expression of at least one target gene.
14. A method according to paragraph 13 wherein the at least one target gene is an endogenous target gene of Notch signalling.
15. A method according to paragraph 14 wherein the at least one target gene is selected from the group consisting of: CBF-1, Hes-1, Hes-5, E(spl), Il-10, CD-23, Dlx-1, CTLA4, CD-4, Numb, Mastermind and Dsh.
16. A method according to any one of paragraphs 13 to 15 wherein the at least one target gene is a reporter gene.
17. A method according to paragraph 16 wherein the at least one target gene is selected from the group consisting of: a gene encoding a polypeptide having an enzymatic activity, a gene comprising a radiolabel or a fluorescent label and a gene encoding a predetermined polypeptide epitope.
18. A method according to any one of paragraphs 13 to 17 wherein the at least one target gene is under the transcriptional control of a promoter region sensitive to Notch signalling.
19. A method according to paragraph 18 wherein the promoter region sensitive to Notch signalling is a CBF-1, Hes-1, Hes-5, E(spl), Il-10, CD-23, Dlx-1, CTLA4, CD-4, Numb, Mastermind or Dsh promoter.
20. A method according to any of paragraphs 13 to 19 wherein the at least one target gene is under the transcriptional control of a promoter region sensitive to:
   i) Notch signalling; and
   ii) a second signal; and/or
   iii) a third signal
wherein the second and third signals are different.
21. A method according to paragraph 20 wherein the second signal results from activation of a signalling pathway specific to cells of the immune system.
22. A method according to paragraph 21 wherein the signalling pathway specific to cells of the immune system is a T cell receptor (TCR) signalling pathway.
23. A method according to paragraph 21 wherein the signalling pathway specific to cells of the immune system is a B cell receptor (BCR) signalling pathway.
24. A method according to paragraph 21 wherein the signalling pathway specific to cells of the immune system is a Toll-like receptor (TLR) signalling pathway.
25. A method according to any one of paragraphs 20 to 24 wherein the third signal is a costimulus specific to cells of the immune system.
26. A method according to paragraph 25 wherein the costimulus is selected from the group consisting of: B7 proteins B7.1-CD80, B7.2-CD86, B7H1, B7H2, B7H3, B7RP1, B7RP2, CTLA4, ICOS, CD2, CD24, CD27, CD30, CD34, CD38, CD40, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, TRANCE/RANK ligands, Fas ligand, MHC class II, DEC205-CD205, CD204-Scavenger receptor, CD14, CD206 (mannose receptor), Toll-like receptors (TLRs), such as TLR 1-9, CD207 (Langerin), CD209 (DC-SIGN), FCγ receptor 2 (CD32), CD64 (FCγ receptor 1), CD68, CD83, CD33, CD54, BDCA-2, BDCA-3, BDCA-4, chemokine receptors, cytokines, growth factors and growth factor receptor agonists, and variants, derivatives, analogues and fragments thereof.
27. A method according to any one of paragraphs 13 to 26 wherein expression of the at least one target gene is monitored with a protein assay.
28. A method according to any of paragraphs 13 to 26 wherein expression of the at least one target gene is monitored with a nucleic acid assay.
29. A method according to any one of the preceding paragraphs wherein Notch signalling is activated by activating Notch, providing a constitutively active truncated form of Notch or providing an active Notch IC domain.
30. A method according to any one of the preceding paragraphs wherein the candidate modulator has a molecular weight of less than about 1000.
31. A method according to any one of the preceding paragraphs wherein the candidate modulator has a molecular weight of less than about 500.
32. A method according to any one of the preceding paragraphs wherein the cell of the immune system is a T cell or T cell progenitor.
33. A method as described in paragraph 32 wherein the T-cell is activated by activation of the T-cell receptor.
34. A method as described in paragraph 33 wherein the T-cell is activated with an antigen or antigenic determinant.
35. A method as described in paragraph 33 wherein the T-cell is activated by an anti-CD3 or anti-TCR antibody
36. A method as described in paragraph 35 wherein the anti-CD3 antibody or anti-TCR antibody is bound to a support.
37. A method as described in paragraph 36 wherein the anti-CD3 antibody or anti-TCR antibody is bound to a particulate support.
38. A method as described in paragraph 33 wherein the T-cell is activated with a calcium ionophore.
39. A method as described in paragraph 33 wherein the T-cell is activated with an activator of protein kinase C or MAP Kinase.
40. A method as described in any one of paragraphs 33 to 39 wherein the T-cell is co-activated
41. A method as described in paragraph 40 wherein the T-cell is co-activated by activation of CD28.
42. A method as described in paragraph 41 wherein the T-cell receptor is co-activated by an anti-CD28 antibody or a CD28 ligand.
43. A method as described in any one of paragraphs 33 to 42 wherein the T-cell is activated by an anti-CD3 or anti-TCR antibody and co-activated by an anti-CD28 antibody or a CD28 ligand.
44. A method according to any one of paragraphs 1 to 31 wherein the cell of the immune system is an antigen presenting cell (APC).
45. A method according to any one of paragraphs 1 to 31 wherein the cell of the immune system is a B-cell.
46. A method according to any one of the preceding paragraphs wherein the immune cell is transfected with an expression vector coding for Notch, a constitutively active truncated form of Notch or a Notch IC domain.
47. A method according to any one of the preceding paragraphs wherein the immune cell is transfected with a Notch reporter construct.
48. A modulator identifiable by a method according to any one of the preceding paragraphs.
49. A modulator identified by a method according to any one of the preceding paragraphs.
50. Use of a modulator according to paragraph 48 or paragraph 49 for the preparation of a medicament for the treatment of a disease or condition of, or related to, the immune system.
51. Use of a modulator according to paragraph 50 wherein the disease is a T-cell mediated disease.
52. Use of a modulator according to paragraph 50 wherein the disease is a B-cell mediated disease.
53. Use of a modulator according to paragraph 50 wherein the disease is an APC mediated disease.
54. A pharmaceutical composition comprising a therapeutically effective amount of at least one modulator according to paragraph 48 or paragraph 49 and a pharmaceutically acceptable carrier, diluent and/or excipient.
55. A method as described in any one of the preceding paragraphs wherein Notch signalling is activated with a Notch ligand.
56. A method as described in any one of the preceding paragraphs wherein the Notch ligand is presented on a cell or cell membrane.
57. A method as described in paragraph 45 wherein the Notch ligand is bound to a support.
58. A particle comprising protein comprising a Delta DSL domain and at least one Delta EGF domain bound to a particulate support matrix.
59. A particle comprising a protein comprising a Delta extracellular domain or an active portion thereof bound to a particulate support matrix.
60. A particle as described in paragraph 58 or paragraph 59 wherein the particulate support matrix is a bead.
61. A particle as described in any one of paragraphs 58 to 60 wherein a plurality of such proteins are bound to the particulate support matrix.
62. A method for detecting genes which are upregulated in an immune cell in response to a combination of Notch signalling and immune cell activation comprising the steps of (in any order):
   (a) activating an immune cell;
   (b) activating Notch signalling in the cell;
   (c) monitoring gene expression; and
   (d) determining which genes are upregulated or downregulated.
63. A method for detecting genes which are more significantly upregulated or downregulated in an immune cell in response to a combination of Notch signalling and immune cell activation than in response to Notch signalling or immune cell activation alone comprising the steps of (in any order):
(a) activating an immune cell;
(b) activating Notch signalling in the cell;
(c) monitoring gene expression;
(d) determining whether gene expression is upregulated or downregulated in the cell; and
(e) comparing gene expression from step (d) with controls in which the cell is not activated or Notch signalling is not activated.
64. A method as described in paragraphs 62 or paragraph 63 wherein gene expression is monitored using a microarray.
65. A method as described in any one of paragraphs 62 to 64 wherein the immune cell is a T-cell.
66. A gene detected by a method as described in any one of paragraphs 62 to 65. 67. An assay comprising the steps of (in any order):
   (a) providing a culture of immune cells;
   (b) transfecting said cells with a Notch signalling reporter construct;
   (c) optionally transfecting said cells with a nucleic acid coding for Notch, a constitutively active truncated form of Notch or a Notch IC domain;
   (d) optionally providing a Notch ligand;
   (e) exposing the cells to one or more compound(s) to be tested; and
   (f) determining the difference in Notch signalling between cells exposed to the compound(s) to be tested and cells not so exposed.
68. An assay comprising the steps of (in any order):
   (a) providing a culture of immune cells;
   (b) optionally transfecting said cells with a Notch signalling reporter construct;
   (c) transfecting said cells with a nucleic acid coding for Notch, a constitutively active truncated form of Notch or a Notch IC domain;
   (d) optionally providing a Notch ligand;
   (e) exposing the cells to one or more compound(s) to be tested; and
   (f) determining the difference in Notch signalling between cells exposed to the compound(s) to be tested and cells not so exposed.
69. An assay as described in paragraph 67 or paragraph 68 comprising the step of activating the immune cell.
70. A method or assay as described in any one of the preceding paragraphs wherein Notch signalling is monitored by monitoring cytokine production.
71. A method or assay as described in paragraph 70 wherein Notch signalling is monitored by monitoring IL-10 production.
72. A method or assay as described in paragraph 70 wherein Notch signalling is monitored by monitoring TNF production.
73. A method or assay as described in paragraph 70 wherein Notch signalling is monitored by monitoring IFN gamma production.
74. A method or assay as described in paragraph 70 wherein Notch signalling is monitored by monitoring IL-5 production.
75. A method or assay as described in paragraph 70 wherein Notch signalling is monitored by monitoring IL-13 production.
76. An immune cell transfected with:
   (i) a Notch signalling reporter construct; and
   (ii) an expression vector coding for Notch, a constitutively active truncated form of Notch or a Notch IC domain.
77. An immune cell transfected with:
   (i) a Notch signalling reporter construct; and
   (ii) an expression vector coding for a constitutively active truncated form of Notch.
78. An immune cell transfected with:
   (i) a Notch signalling reporter construct; and
   (ii) an expression vector coding for a Notch IC domain.
79. An immune cell as described in any one of paragraphs 76 to 78 which is stably transfected.
80. A method for detecting modulators of Notch signalling comprising the steps of monitoring Notch signalling in a cell of the immune system in the presence and absence of a candidate modulator having a molecular weight of less than about 1000, and determining whether the candidate modulator modulates Notch signalling.
81. A method for detecting modulators of Notch signalling comprising the steps of:
   (a) contacting a cell of the immune system with a candidate modulator having a molecular weight of less than about 1000;
   (b) monitoring Notch signalling; and
   (c) determining whether the candidate modulator modulates Notch signalling.
82. A method as described in paragraph 80 or paragraph 81 wherein the candidate modulator has a molecular weight of less than about 500.
   References (Herein Incorporated by Reference)
Tamura et al. (1995) Curr. Biol. 5:1416-1423.
Artavanis-Tsakonas et al. (1995) Science 268:225-232.
Artavanis-Tsakonas et al. (1999) Science 284:770-776.
Lieber et al. (1993) Genes Dev 7(10):1949-65.
Schroeter et al. (1998) Nature 393(6683):382-6.
Struhl et al. (1998) Cell 93(4):649-60.
Weinmaster(2000) Curr. Opin. Genet. Dev. 10:363-369.
Lu et al. (1996) Proc Natl Acad Sci 93(11):5663-7.
Munro and Freeman (2000) Curr. Biol. 10:813-820.
Ju et al. (2000) Nature 405:191-195.
Moloney et al. (2000) Nature 406:369-375.
Brucker et al. (2000) Nature 406:411-415.
Panin et al. (1997) Nature 387:908-912.
Hicks et al. (2000) Nat. Cell. Biol. 2:515-520.
Irvine (1999) Curr. Opin. Genet. Devel. 9:434-441.
Devereux et al. (1984) Nucleic Acid Research 12:87.
Atschul et al. (1990) J. Mol. Biol. 403-410.
Inaba et al. (1992) J. Exp. Med. 175:1157-1167.
Caux et al. (1992) Nature 360:258-261.
Coffin et al. (1998) Gene Therapy 5:718-722.
Kroll et al. (1993) DNA Cell Biol. 12:441-453.
Osborne and Miele (1999) Immunity 11:653-663.
Matsuno et al. (1995) Development 121(8):2633-44.
Matsuno et al. (1998) Nat. Genet. 19:74-78.
Ordentlich et al. (1998) Mol. Cell. Biol. 18:2230-2239.
Takebayashi et al. (1994) J Biol Chem 269(7):150-6.
Leimeister et al. (1999) Mech Dev 85(1-2):173-7.
Maddox (1983) J. Exp. Med. 15(8):121 1
Sauber et al (1995) Virol. 213:439-449
Chee et al. (1996) Science 274:601-614.
Camilli et al. (1994) Proc Natl Acad Sci USA 91:2634-2638.
Hoyne et al. (2000) Immunology 100:281-288.
Hoyne et al. (2001) Immunol Rev 182:215-27.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1. A method for detecting modulators of Notch or immune signalling comprising the steps of (in any order):
(a) activating Notch signalling in a cell of the immune system;
(b) contacting the cell with a candidate modulator of Notch or immune signalling;
(c) monitoring Notch or immune signalling; and
(d) determining whether the candidate modulator modulates Notch or immune signalling.

2. A method for detecting modulators of Notch or immune signalling comprising the steps of (in any order):
(a) activating a cell of the immune system;
(b) contacting the cell with a candidate modulator of Notch or immune signalling;
(c) monitoring Notch or immune signalling; and
(d) determining whether the candidate modulator modulates Notch or immune signalling.

3. A method for detecting modulators of Notch or immune signalling comprising the steps of (in any order):
(a) activating a cell of the immune system;
(b) activating Notch signalling in the cell;
(c) contacting the cell with a candidate modulator of Notch or immune signalling;
(d) monitoring Notch or immune signalling; and
(e) determining whether the candidate modulator modulates Notch or immune signalling.

4. A method for detecting modulators of Notch signalling comprising the steps of (in any order):
(a) activating Notch signalling in a cell of the immune system;
(b) contacting the cell with a candidate modulator of Notch signalling;
(c) monitoring Notch or immune signalling; and
(d) determining whether the candidate modulator modulates Notch or immune signalling.

5. A method for detecting modulators of Notch signalling comprising the steps of (in any order):
(a) activating a cell of the immune system;
(b) contacting the cell with a candidate modulator of Notch signalling;
(c) monitoring Notch or immune signalling; and
(d) determining whether the candidate modulator modulates Notch or immune signalling.

6. A method for detecting modulators of Notch signalling comprising the steps of (in any order):
(a) activating a cell of the immune system;
(b) activating Notch signalling in the cell;
(c) contacting the cell with a candidate modulator of Notch signalling;
(d) monitoring Notch or immune signalling; and
(e) determining whether the candidate modulator modulates Notch or immune signalling.

7. The method of claim 1, wherein step (b) comprises contacting the cell with a candidate modulator of Notch signalling.

8. The method of claim 1, wherein step (c) comprises monitoring Notch signalling.

9. The method of claim 1, wherein step (d) comprisies determining whether the candidate modulator modulates Notch signalling.

10. The method of claim 1, wherein immune cell activation is at least 20% optimal with respect to Notch or immune signalling.

11. The method of claim 1, wherein immune cell activation is at least 70% optimal with respect to Notch or immune signalling.

12. The method of claim 1, wherein the candidate modulator is selected from the group consisting of an organic compound, an inorganic compound, a peptide, a polypeptide, a polynucleotide, an antibody, a fragment of an antibody, a cytokine and a fragment of a cytokine.

13. The method of claim 1, wherein monitoring Notch signalling comprises monitoring expression levels of at least one target gene.

14. The method of claim 13, wherein the at least one target gene is an endogenous target gene of Notch signalling.

15. The method of claim 13, wherein the at least one target gene is selected from the group consisting of CBF-1, Hes-1, Hes-5, E(spl), IL-10, CD-23, Dlx-1, CTLA4, CD-4, Numb, Mastermind and Dsh.

16. The method of claim 13, wherein the at least one target gene is a reporter gene.

17. The method of claim 16, wherein the reporter gene is selected from the group consisting of a gene encoding a polypeptide having an enzymatic activity, a gene comprising a radiolabel or a fluorescent label, and a gene encoding a predetermined polypeptide epitope.

18. The method of claim 13, wherein the at least one target gene is under transcriptional control of a promoter region sensitive to Notch signalling.

19. The method of claim 18, wherein the promoter region sensitive to Notch signalling is selected from the group consisting of CBF-1, Hes-1, Hes-5, E(spl), IL-10, CD-23, Dlx-1, CTLA4, CD-4, Numb, Mastermind and Dsh promoters.

20. The method of claim 13, wherein the at least one target gene is under transcriptional control of a promoter region sensitive to i) Notch signalling; and ii) a second signal.

21. The method of claim 20, wherein the promoter region is sensitive to iii) a third signal.

22. The method of claim 20, wherein the second signal results from activation of a signalling pathway specific to cells of the immune system.

23. The method of claim 22, wherein the signalling pathway specific to cells of the immune system is a T cell receptor (TCR) signalling pathway.

24. The method of claim 22, wherein the signalling pathway specific to cells of the immune system is a B cell receptor (BCR) signalling pathway.

25. The method of claim 22, wherein the signalling pathway specific to cells of the immune system is a Toll-like receptor (TLR) signalling pathway.

26. The method of claim 21, wherein the third signal is a co-stimulus specific to cells of the immune system.

27. The method of claim 26, wherein the co-stimulus is selected from the group consisting of B7 proteins, CTLA4, ICOS, CD2, CD24, CD27, CD30, CD34, CD38, CD40, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, TRANCE/RANK ligands, Fas ligand, MHC class II, DEC205-CD205, CD204-Scavenger receptor, C 14, CD206 (mannose receptor), Toll-like receptors (TLRs), CD207 (Langerin), CD209 (DC-SIGN), FCγ receptor 2 (CD32), CD64 (FCγ receptor 1), CD68, CD83, CD33, CD54, BDCA-2, BDCA-3, BDCA-4, chemokine receptors, cytokines, growth factors, growth factor receptor agonists, and variants, derivatives, analogues and fragments thereof.

28. The method of claim 27, wherein the B7 protein is B7.1-CD80, B7.2-CD86, B7H1, B7H2, B7H3, B7RP1 or B7RP2.

29. The method of claim 13, wherein expression of the at least one target gene is monitored with a protein assay.

30. The method of claim 13, wherein expression of the at least one target gene is monitored with a nucleic acid assay.

31. The method of claim 1, wherein Notch signalling is activated by (i) activating Notch, (ii) providing a constitutively active truncated form of Notch, or (iii) providing an active Notch IC domain.

32. The method of claim 1, wherein the candidate modulator has a molecular weight of less than about 1000.

33. The method of claim 1, wherein the candidate modulator has a molecular weight of less than about 500.

34. The method of claim 1, wherein the cell of the immune system is a T cell or T cell progenitor.

35. The method of claim 34, wherein the T-cell is activated by activation of a T-cell receptor.

36. The method of claim 34, wherein the T-cell is activated with an antigen or antigenic determinant.

37. The method of claim 34, wherein the T-cell is activated by an anti-CD3 antibody or an anti-TCR antibody

38. The method of claim 37, wherein the anti-CD3 antibody or anti-TCR antibody is bound to a support.

39. The method of claim 38, wherein the support is a particulate support.

40. The method of claim 34, wherein the T-cell is activated with a calcium ionophore.

41. The method of claim 34, wherein the T-cell is activated with an activator of protein kinase C or MAP Kinase.

42. The method of claim 34, wherein the T-cell is co-activated

43. The method of claim 42, wherein the T-cell is co-activated by activation of CD28.

44. The method of claim 43, wherein activation of CD28 is by an anti-CD28 antibody or a CD28 ligand.

45. The method of claim 42, wherein the T-cell is activated by an anti-CD3 antibody or and an anti-TCR antibody, and co-activated by an anti-CD28 antibody or a CD28 ligand.

46. The method of claim 1, wherein the cell of the immune system is an antigen presenting cell (APC).

47. The method of claim 1, wherein the cell of the immune system is a B-cell.

48. The method of claim 1, wherein the immune cell is transfected with an expression vector encoding (i) Notch, (ii) a constitutively active truncated form of Notch, or (iii) a Notch IC domain.

49. The method of claim 1, wherein the immune cell is transfected with a Notch reporter construct.

50. A modulator of Notch identified by the method of claim 1.

51. A composition comprising a therapeutically effective amount of at least one modulator according to claim 50 and a pharmaceutically acceptable carrier, diluent and/or excipient.

52. A method of treating a disease or condition of, or related to, the immune system comprising administering the composition of claim 51 to a subject in need thereof.

53. The method of claim 52, wherein the disease is a T-cell mediated disease.

54. The method of claim 52, wherein the disease is a B-cell mediated disease.

55. The method of claim 52, wherein the disease is an APC mediated disease.

56. The method of claim 1, wherein Notch signalling is activated with a Notch ligand.

57. The method of claim 56, wherein the Notch ligand is presented on a cell or cell membrane.

58. The method of claim 56, wherein the Notch ligand is bound to a support.

59. A particle comprising protein comprising a Delta DSL domain and at least one Delta EGF domain bound to a particulate support matrix.

60. A particle comprising a protein comprising a Delta extracellular domain, or an active portion thereof, bound to a particulate support matrix.

61. The particle of claim 59, wherein the particulate support matrix is a bead.

62. The particle of claim 60, wherein the particulate support matrix is a bead.

63. The particle of claim 59, wherein a plurality of proteins comprising a Delta DSL domain and at least one Delta EGF domain are bound to the particulate support matrix.

64. The particle of claim 60, wherein a plurality of proteins comprising a Delta extracellular domain, or an active portion thereof, are bound to the particulate support matrix.

65. A method for identifying genes which are upregulated in an immune cell in response to a combination of Notch signalling and immune cell activation comprising the steps of (in any order):
(a) activating an immune cell;
(b) activating Notch signalling in the cell;
(c) monitoring gene expression; and
(d) determining which genes are upregulated,
thereby identifying genes which are upregulated in an immune cell in response to a combination of Notch signalling and immune cell activation.

66. A method for identifying genes which are upregulated or downregulated in an immune cell to a greater extent in response to a combination of Notch signalling and immune cell activation than in response to Notch signalling or immune cell activation alone, the method comprising the steps of (in any order):
(a) activating an immune cell;
(b) activating Notch signalling in the cell;
(c) monitoring gene expression;
(d) determining whether gene expression is upregulated or downregulated in the cell; and
(e) comparing gene expression from step (d) with gene expression in a cell that is not activated or wherein Notch signalling is not activated,
thereby identifying genes which are upregulated or downregulated in an immune cell to a greater extent in response to a combination of Notch signalling and immune cell activation than in response to Notch signalling or immune cell activation alone.

67. The method of claim 65, wherein gene expression is monitored using a microarray.

68. The method of claim 65, wherein the immune cell is a T-cell.

69. A gene identified by the method of claim 65.

70. An assay for identifying a compound that modulates Notch siganlling comprising the steps of (in any order):
(a) providing a culture of immune cells;
(b) transfecting said cells with a Notch signalling reporter construct;
(c) optionally transfecting said cells with a nucleic acid encoding (i) Notch, (ii) a constitutively active truncated form of Notch or (iii) a Notch IC domain;
(d) optionally providing a Notch ligand;
(e) exposing the cells to at least one compound to be tested; and
(f) determining the difference in Notch signalling between cells exposed to the compound to be tested and cells not exposed to the compound,
thereby identifying a compound that modulates Notch signalling.

71. An assay for identifying a compound that modulates Notch siganlling comprising the steps of (in any order):
(a) providing a culture of immune cells;
(b) optionally transfecting said cells with a Notch signalling reporter construct;
(c) transfecting said cells with (i) a nucleic acid encoding Notch, (ii) a constitutively active truncated form of Notch or (iii) a Notch IC domain;
(d) optionally providing a Notch ligand;
(e) exposing the cells to at least one compound to be tested; and
(f) determining the difference in Notch signalling between cells exposed to the compound to be tested and cells not exposed to the compound,
thereby identifying a compound that modulates Notch signalling.

72. The assay of claim 70, further comprising the step of activating the immune cell.

73. The method of claim 65, wherein Notch signalling is monitored by monitoring cytokine production.

74. The method of claim 65, wherein Notch signalling is monitored by monitoring IL-10 production.

75. The method of claim 65, wherein Notch signalling is monitored by monitoring TNF production.

76. The method of claim 65, wherein Notch signalling is monitored by monitoring IFN gamma production.

77. The method of claim 65, wherein Notch signalling is monitored by monitoring IL-5 production.

78. The method of claim 65, wherein Notch signalling is monitored by monitoring IL-13 production.

79. An immune cell transfected with:
(a) a Notch signalling reporter construct; and
(b) (i) an expression vector encoding Notch, (ii) a constitutively active truncated form of Notch or (iii) a Notch IC domain.

80. The immune cell of claim 79, wherein the cell is transfected with an expression vector encoding a constitutively active truncated form of Notch.

81. The immune cell of claim 79, wherein the cell is transfected with an expression vector coding for a Notch IC domain.

82. The immune cell of claim 79, wherein the cell is stably transfected.

83. A method for identifying a modulator of Notch signalling comprising the steps of
(a) monitoring Notch signalling in a cell of the immune system in the presence and absence of a candidate modulator having a molecular weight of less than about 1000, and
(b) determining whether the candidate modulator modulates Notch signalling,
thereby identifying a modulator of Notch signalling.

84. The method of claim 83, wherein the candidate modulator has a molecular weight of less than about 500.
